(19) United States
(12) Patent Application Publication
(10) Pub. No.: US 2007/0004680 A1
(43) Pub. Date: Jan. 4, 2007

(54) Compositions and methods for modulating gated ion channels

(75) Inventors: Kazimierz Babinski, Dorval (CA); Walter A. Szarek, Kingston (CA); Rahul Vohra, Kanata (CA); Thomas Varming, Charlottenlund (DK); Philip K. Ahring, Bagsvaerd (DK); Tino Dyhring Joergensen, Solroed Strand (DK); Gordon John Blackburn-Munro, Herlev (DK)

Correspondence Address: LAHIVE & COCKFIELD, LLP, ONE POST OFFICE SQUARE, BOSTON, MA, 02109-2127, US

(73) Assignee: PainCeptor Pharma Corporation, St. Laurent (CA)

(21) Appl. No.: 11/096,239

(22) Filed: Mar. 30, 2005

Related U.S. Application Data
(60) Provisional application No. 60/558,059, filed on Mar. 30, 2004.
(60) Provisional application No. 60/564,063, filed on Apr. 20, 2004.

(51) Int. Cl. A61K 31/675; A61K 31/5377; A61K 31/496; A61K 31/4709

(52) U.S. Cl. 514/81; 514/233.5; 514/248; 514/253.05; 514/253.06; 514/310; 514/314

(57) ABSTRACT

The present invention relates to compositions and methods to modulate the activity of gated ion channels.

### RELATED APPLICATIONS

This application claims priority to U.S. Provisional Application No. 60/558,059 filed Mar. 30, 2004, entitled “COMPOSITIONS AND METHODS FOR NEUROLOGICAL CONDITIONS AND PAIN,” and U.S. Provisional Application No. 60/564,063, filed April 20, 2004, entitled “COMPOSITIONS AND METHODS FOR NEUROLOGICAL CONDITIONS AND PAIN.” The entire contents of each of the aforementioned applications are hereby expressly incorporated herein by reference in their entireties.

### TECHNICAL FIELD

The present invention relates to compositions which modulate the activity of gated ion channels and methods and uses thereof.

### BACKGROUND

Mammalian cell membranes are important to the structural integrity and activity of many cells and tissues. Of particular interest is the study of trans-membrane gated ion channels which act to directly and indirectly control a variety of pharmacological, physiological, and cellular processes. Numerous gated ion channels have been identified and investigated to determine their roles in cell function.

Gated ion channels are involved in receiving, integrating, transducing, conducting, and transmitting signals in a cell, e.g., a neuronal or muscle cell. Gated ion channels can determine membrane excitability. Gated ion channels can also influence the resting potential of membranes, wave forms, and frequencies of action potentials, and thresholds of excitation. Gated ion channels are typically expressed in electrically excitable cells, e.g., neuronal cells, and are multimeric. Gated ion channels may also be found in nonexcitable cells (e.g., adipose cells or liver cells), where they may play a role in, for example, signal transduction.

Among the numerous gated ion channels identified to date are channels that are responsive to, for example, modulation of voltage, temperature, chemical environment, pH, ligand concentration and/or mechanical stimulation. Examples of specific modulators include, ATP, capsaicin, neurotransmitters (e.g., acetylcholine), ions, e.g., Na⁺, Ca⁺, K⁺, Cl⁻, H⁺, Zn⁺, Cd⁺, and/or peptides, e.g., FMRF. Examples of gated ion channels responsive to these stimuli are members of the DEG/ENaC, TRPV and P2X gene superfamilies.

Members of the DEG/ENaC gene superfamily show a high degree of functional heterogeneity with a wide tissue distribution that includes transporting epithelia as well as neuronal excitable tissues. DEG/ENaC proteins are membrane proteins which are characterized by two transmembrane spanning domains, intracellular N- and C-termini and a cysteine-rich extracellular loop. Depending on their function in the cell, DEG/ENaC channels are either constitutively active like epithelial sodium channels (ENaC) which are involved in sodium homeostasis, or activated by mechanical stimuli as postulated for C. elegans degnerins, or by ligands such as peptides as is the case for FaNaC from Helix aspersa which is a FMRF amide peptide-activated channel and is involved in neurotransmission, or by protons as in the case for the acid sensing ion channels (ASICs). The mammalian members of this gene family known to date are αENaC (also known as SCNN1A or scnn1A), βENaC (also known as SCNN1B or scnn1B), }ENaC (also known as SCNN1G or scnn1G), δENaC (also known as ENaCd, SCNN1D, scnn1D and dNaCh), ASIC1a (also known as ASIC, ASIC1, BNaC2, hBNaC2, ASICalpha, ACCN2 and accn2), ASIC1b (also known as ASICbeta), ASIC2a (also known as MDEG2, ASIC2b), ASIC2b (also known as MDEG2, ASIC2b), ASIC3 (also known as hASIC3, DRASIC, TNaC1, SLNAC1, ACCN3 and accn3), ASIC4 (also known as BNaC4, SPASIC, ACCN4 and accn4), BLINaC (also known as hINaC, ACCN5 and accn5), and hINaC. For a recent review on this gene superfamily see Kellenberger, S. and Schild, L. (2002) Physiol. Rev. 82:735, incorporated herein by reference.

There are seven presently known members of the P2X gene superfamily; P2X₁ (also known as P2RX1), P2X₂ (also known as P2RX2), P2X₃ (also known as P2RX3), P2X₄ (also known as P2RX4), P2X₅ (also known as P2RX5), P2X₆ (also known as P2RX6), and P2X₇ (also known as P2RX7). P2X protein structure is similar to ASIC protein structure in that they contain two transmembrane spanning domains, intracellular N- and C-termini and a cysteine-rich extracellular loop. All P2X receptors open in response to the release of extracellular ATP and are permeable to small ions and some have significant calcium permeability. P2X receptors are abundantly distributed on neurons, glia, epithelial, endothelia, bone, muscle and hematopoietic tissues. For a recent review on this gene superfamily, see North, R. A. (2002) Physiol. Rev. 82:1013, incorporated herein by reference.

The receptor expressed in sensory neurons that reacts to the pungent ingredient in chili peppers to produce a burning pain is the capsaicin (TRPV or vanilloid) receptor, denoted TRPV1 (also known as VR1, TRPV1alpha, TRPV1beta). The TRPV1 receptor forms a nonselective cation channel that is activated by both capsaicin and resiniferatoxin (RTX) as well as noxious heat (>43° C.), with the evoked responses potentiated by protons, e.g., H⁺ ions. Acid pH is also capable of inducing a slowly inactivating current that resembles the native proton-sensitive current in dorsal root ganglia. Expression of TRPV1, although predominantly in primary sensory neurons, is also found in various brain nuclei and the spinal cord (Physiol. Genomics 4:165-174, 2001).

Two structurally related receptors, TRPV2 (also known as VRL1 and VRL) and TRPV4 (also known as VRL-2, Trp12, VROAC, OTRPC4), do not respond to capsaicin, acid or moderate heat but rather are activated by high temperatures (Caterina, M. J., et al. (1999) Nature. 398(6726):436-41). In addition, this family of receptors, e.g., the TRPV or vanilloid family, contains the ECAC-1 (also known as TRPV5 and CAT2, CaT2) and ECAC-2 (also known as TRPV6, CaT, ECaC, CAT1, CATL, and OTRPC3) receptors which are calcium selective channels (Peng, J. B., et al. (2001) Genomics 76(1-3):99-109). For a recent review of TRPV (vanilloid) receptors, see Szallasi, A. and Blumberg, P. M. (1999) Pharmacol. Rev. 51:159, incorporated herein by reference.

The ability of the members of the gated ion channels to respond to various stimuli, for example, chemical (e.g., ions), thermal and mechanical stimuli, and their location throughout the body, e.g., small diameter primary sensory neurons in the dorsal root ganglia and trigeminal ganglia, as well data derived from in vitro and in vivo models has implicated these channels in numerous neurological diseases, disorders and conditions. For example, it has been shown that the rat ASIC2a channel is activated by the same mutations as those causing neuronal degeneration in C. elegans. In addition, these receptors are activated by increases in extracellular proton, e.g., H+, concentration. By infusing low pH solutions into skin or muscle as well as prolonged intradermal infusion of low pH solutions creates a change in extracellular pH that mimics the hyperalgesia of chronic pain. Furthermore, transgenic mice, e.g., ASIC2a, ASIC3, P2X3 transgenic mice, all have modified responses to noxious and non-noxious stimuli. Thus, the biophysical, anatomical and pharmacological properties of the gated ion channels are consistent with their involvement in nociception.

Research has shown that ASICs play a role in pain, neurological diseases and disorders, gastrointestinal diseases and disorders, genitourinary diseases and disorders, and inflammation. For example, it has been shown that ASICs play a role in pain sensation (Price, M. P. et al., Neuron. 2001; 32(6): 1071-83; Chen, C.-C. et al., Neurobiology 2002; 99(13) 8992-8997), including visceral and somatic pain (Aziz, Q., Eur. J. Gastroenterol. Hepatol. 2001; 13(8):891-6); chest pain that accompanies cardiac ischemia (Mamet, J. et al., J. Neurosci. 2002; 22(24): 10662-70), and chronic hyperalgesia (Sluka, K. A. et al., Pain. 2003; 106(3):229-39). ASICs in central neurons have been shown to possibly contribute to the neuronal cell death associated with brain ischemia and epilepsy (Chesler, M., Physiol. Rev. 2003; 83: 1183-1221; Lipton, P., Physiol. Rev. 1999; 79:1431-1568). ASICs have also been shown to contribute to the neural mechanisms of fear conditioning, synaptic plasticity, learning, and memory (Wemmie, J. et al., J. Neurosci. 2003; 23(13):5496-5502; Wemmie, J. et al., Neuron. 2002; 34(3):463-77). ASICs have been shown to be involved in inflammation-related persistant pain and inflamed intestine (Wu, L. J. et al., J. Biol. Chem. 2004; 279(42):43716-24; Yiangou, Y., et al., Eur. J. Gastroenterol. Hepatol. 2001; 13(8): 891-6), and gastrointestinal stasis (Holzer, Curr. Opin. Pharm. 2003; 3: 618-325). Recent studies done in humans indicate that ASICs are the primary sensors of acid-induced pain (Ugawa et al., J. Clin. Invest. 2002; 110: 1185-90; Jones et al, J. Neurosci. 2004; 24: 10974-9). Futhermore, ASICs are also thought to play a role in gametogenesis and early embryonic development in Drosophila (Darboux, I. et al, J. Biol. Chem. 1998; 273(16):9424-9), underlie mechanosensory function in the gut (Page, A. J. et al Gastroenterology. 2004; 127(6):1739-47), and have been shown to be involved in endocrine glands (Grunder, S. et al, Neuroreport. 2000; 11(8): 1607-11). Therefore, compounds that modulate these gated ion channels would be useful in the treatment of such diseases and disorders.

### SUMMARY OF THE INVENTION

In one aspect, the invention provides a method of modulating the activity of a gated ion channel, comprising contacting a cell expressing a gated ion channel with an effective amount of a compound represented by the Formula 1, 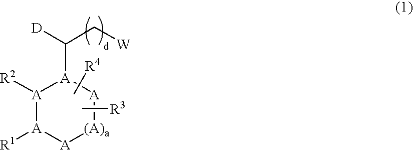
or a pharmaceutically acceptable salt thereof, wherein A is, independently, either an sp²- or sp³-hybridized carbon or nitrogen atom; D is selected from the group consisting of —H, —OH, halogen, —(CH₂)₀₋₆Y, —O—(CH₂)₀₋₆Y, wherein Y is selected from —H, —CN, —CO₂H, —SO₃H, —SO₂H, —PO₃H₂, —NO₂, —SSO₃H, halomethyl, dihalomethyl, trihalomethyl, N-methyl-piperidinyl, morpholinyl, hydroxyphenyl, phenyl, piperazinyl, cyclopropyl, cyclopentyl, cyclohexyl, pyridinyl, 5H-tetrazolyl, triazolyl or piperidinyl; R⁹(CH₂)₀₋₆COO—, —N(R⁹)(CH₂)₀₋₆COO(R⁹), —O(CH₂)₀₋₆(R⁹), —(CH₂)₁₋₆COO(R⁹), —(CH₂)₀₋₆N(R⁹)COO(R⁹), —(CH₂)₁₋₆N(R⁹)CO(R⁹), —(CH₂)₁₋₆CONH(R⁹), ═NOR⁹, wherein R⁹ is selected from the group consisting of —H, —C₁₋₄-alkyl, N-methyl-piperidinyl, morpholinyl, hydroxyphenyl, phenyl, piperazinyl, cyclopropyl, cyclopentyl, cyclohexyl, pyridinyl, 5H-tetrazolyl, triazolyl, or piperidinyl; —N(X¹)X², —SO₂N(X¹)X², wherein X¹ and X² are each, independently, H, aryl, or C₁-C₆-alkyl; salts thereof, esters thereof, and any combination thereof; W is selected from the group consisting of —H, —OH, halogen, —(CH₂)₀₋₆Y, —O—(CH₂)₀₋₆Y, wherein Y is selected from —H, —CN—CO₂H, —SO₃H, —SO₂H, —PO₃H₂, —NO₂, —SSO₃H, halomethyl, dihalomethyl, trihalomethyl, N-methyl-piperidinyl, morpholinyl, hydroxyphenyl, phenyl, piperazinyl, cyclopropyl, cyclopentyl, cyclohexyl, pyridinyl, 5H-tetrazolyl, triazolyl or piperidinyl; R⁹(CH₂)₀₋₆COO—, —N(R⁹)(CH₂)₀₋₆COO(R⁹), —O(CH₂)₀₋₆(R⁹), —(CH₂)₁₋₆COO(R⁹), —(CH₂)₁₋₆N(R⁹)COO(R⁹), —(CH₂)₁₋₆N(R⁹)CO(R⁹), —(CH₂)₁₋₆CONH(R⁹), ═NOR⁹, wherein R⁹ is selected from the group consisting of —H, —C₁₋₄-alkyl, N-methyl-piperidinyl, morpholinyl, hydroxyphenyl, phenyl, piperazinyl, cyclopropyl, cyclopentyl, cyclohexyl, pyridinyl, 5H-tetrazolyl, triazolyl, or piperidinyl; —N(X¹)X², —SO₂N(X¹)X², or —(CH₂)₁₋₆SO₂N(X¹)X², wherein X¹ and X² are each, independently, H, aryl, C₁-C₆-alkyl; salts thereof, esters thereof, and any combination thereof; and any combination thereof; or the formula 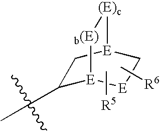
wherein E is, independently, either an sp²- or sp³-hybridized carbon or nitrogen atom; R⁵ and R⁶ are each, independently, —H, —OH, —(CH₂)₀₋₆Y, —O—(CH₂)₀₋₆Y, wherein Y is selected from —H, —OH, —CN, halo, —CHCH₂, —CH₂CHCH₂, —NO₂, morpholinyl, hydroxyphenyl, phenyl, piperazinyl, cyclopropyl, cyclopentyl, cyclohexyl, pyridinyl, 5H-tetrazolyl, triazolyl, piperidinyl, alkylcarbonyl, alkylthiocarbonyl, alkoxycarbonyl, aminocarbonyl, —CO₂H; —SO₃H; —SO₂H; —SO₂NH₂, —SSO₃H, —PO₃H₂; —NO₂, —SH, —OSO₃H, —OC(O)(OH), —O—, —S—, halomethyl, dihalomethyl, trihalomethyl, —SO₂N(X¹)X² or N(X¹)X², wherein X¹ and X² are each, independently, H, aryl, C₁-C₆-alkyl, esters thereof; salts thereof, and any combination thereof; R¹, R², R³, R⁴, are each, independently, selected from the group consisting of —H, —OH, halogen, —(CH₂)₀₋₆Y, —O—(CH₂)₀₋₆Y, wherein Y is selected from —H, —CN, —CO₂H, —SO₃H, —SO₂H, —PO₃H₂, —NO₂, —SSO₃H, halomethyl, dihalomethyl, trihalomethyl, N-methyl-piperidinyl, morpholinyl, hydroxyphenyl, phenyl, piperazinyl, cyclopropyl, cyclopentyl, cyclohexyl, pyridinyl, 5H-tetrazolyl, triazolyl or piperidinyl; R⁹(CH₂)₀₋₆COO—, —N(R⁹)(CH₂)₀₋₆COO(R⁹), —O(CH₂)₀₋₆(R⁹), —(CH₂)₁₋₆COO(R⁹), —(CH₂)₁₋₆N(R⁹)COO(R⁹), —(CH₂)₁₋₆N(R⁹)CO(R⁹), —(CH₂)₁₋₆CONH(R⁹), ═NOR⁹, wherein R⁹ is selected from the group consisting of —H, —C₁₋₄-alkyl, N-methyl-piperidinyl, morpholinyl, hydroxyphenyl, phenyl, piperazinyl, cyclopropyl, cyclopentyl, cyclohexyl, pyridinyl, 5H-tetrazolyl, triazolyl, or piperidinyl; —N(X¹)X², —SO₂N(X¹)X² wherein X¹ and X² are each, independently, H, aryl, C₁-C₆-alkyl; salts thereof, esters thereof, and any combination thereof; R¹ and R² can also form together for a fused 5- or 6-membered ring composed of one of the following bridging bivalent radicals (reading from R¹ to R²): -GR¹⁰—CH₂—CH₂—, —CH₂-GR¹⁰—CH₂—, —CH₂—CH₂-GR¹⁰—, -GR¹⁰—CH₂—CH₂—CH₂—, —CH₂-GR¹⁰—CH₂—CH₂—, —CH₂—CH₂-GR¹⁰—CH₂—, —CH₂—CH₂—CH₂—GR¹⁰—, -GR¹⁰═CH—CH═CH—, —CH=GR¹⁰—CH═CH—, —CH═CH-GR¹⁰═CH—, —CH═CH—CH=GR¹⁰—; wherein G is either an sp²- or sp³-hybridized carbon or nitrogen atom; wherein R¹⁰ has the meaning set forth for R⁶ and a, b, c and d are each 0 or 1.

In certain embodiments of Formula 1, W is 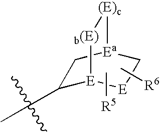
wherein E^{a} is N, E is C, R⁶ is H, and R⁵ is —CHCH₂ or —CH₂CH₃.

In other embodiments of Formula 1, R¹ and R² form together for a fused 5- or 6-membered ring composed of one of the following bridging bivalent radicals (reading from R¹ to R²): -GR¹⁰—CH₂—CH₂—, —CH₂-GR¹⁰—CH₂—, —CH₂—CH₂-GR¹⁰—, —CH=GR¹⁰—CH═CH—, —CH═CH-GR¹⁰═CH—. In a preferred embodiment of Formula 1, a is 1.

In other embodiments, the compound of the invention is represented by the Formula 2, 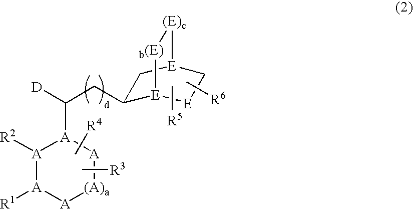
or a pharmaceutically acceptable salt thereof, wherein A and E are each, independently, an sp²- or sp³-hybridized carbon or nitrogen atom; D is selected from the group consisting of —H, —OH, halogen, —(CH₂)₀₋₆Y, —O—(CH₂)₀₋₆Y, wherein Y is selected from —H, —CN, —CO₂H, —SO₃H, —SO₂H, —PO₃H₂, —NO₂, —SSO₃H, halomethyl, dihalomethyl, trihalomethyl, N-methyl-piperidinyl, morpholinyl, hydroxyphenyl, phenyl, piperazinyl, cyclopropyl, cyclopentyl, cyclohexyl, pyridinyl, 5H-tetrazolyl, triazolyl or piperidinyl; R⁹(CH₂)₀₋₆COO—, —N(R⁹)(CH₂)₀₋₆COO(R⁹), —O(CH₂)₀₋₆(R⁹), —(CH₂)₁₋₆N(R⁹)COO(R⁹), —(CH₂)₁₋₆N(R⁹)CO(R⁹), —(CH₂)₁₋₆CONH(R⁹), ═NOR⁹, wherein R⁹ is selected from the group consisting of —H, —C₁₋₄-alkyl, N-methyl-piperidinyl, morpholinyl, hydroxyphenyl, phenyl, piperazinyl, cyclopropyl, cyclopentyl, cyclohexyl, pyridinyl, 5H-tetrazolyl, triazolyl, or piperidinyl; —N(X¹)X², —SO₂N(X¹)X², wherein X¹ and X² are each, independently, H, aryl, C₁-C₆-alkyl; salts thereof, esters thereof, and any combination thereof; R¹, R², R³, R⁴, R⁵ and R⁶, are each, independently, —H, —OH, halo, C₁-C₆-alkyl, —O—C₁₋₆-alkyl, —CHCH₂, —CH₂CHCH₂, —(CH₂)₀₋₆Y, —O—(CH₂)₀₋₆Y, wherein Y is selected from —H, —CN, —CO₂H, —SO₃H, —SO₂H, —PO₃H₂, —NO₂, —SSO₃H, halomethyl, dihalomethyl, trihalomethyl, N-methyl-piperidinyl, morpholinyl, hydroxyphenyl, phenyl, piperazinyl, cyclopropyl, cyclopentyl, cyclohexyl, pyridinyl, 5H-tetrazolyl, triazolyl or piperidinyl; R⁹(CH₂)₀₋₆COO—, —N(R⁹)(CH₂)₀₋₆COO(R⁹), —O(CH₂)₀₋₆(R⁹), —(CH₂)₁₋₆COO(R⁹), —(CH₂)₁₋₆N(R⁹)COO(R⁹), —(CH₂)₁₋₆N(R⁹)CO(R⁹), —(CH₂)₁₋₆CONH(R⁹), ═NOR⁹, wherein R⁹ is selected from the group consisting of —H, —C₁₋₄-alkyl, N-methyl-piperidinyl, morpholinyl, hydroxyphenyl, phenyl, piperazinyl, cyclopropyl, cyclopentyl, cyclohexyl, pyridinyl, 5H-tetrazolyl, triazolyl, or piperidinyl; —N(X¹)X², —SO₂N(X¹)X² wherein X¹ and X² are each, independently, H, aryl, C₁-C₆-alkyl; salts thereof, esters thereof, and any combination thereof; R¹ and R² can also form together for a fused 5- or 6-membered ring composed of one of the following bridging bivalent radicals (reading from R¹ to R²): -GR¹⁰—CH₂—CH₂—, —CH₂-GR¹⁰—CH₂—, —CH₂—CH₂-GR¹⁰—, -GR¹⁰—CH₂—CH₂—CH₂—, —CH₂-GR¹⁰—CH₂—CH₂—, —CH₂—CH₂-GR¹⁰—CH₂—, —CH₂—CH₂—CH₂-GR¹⁰—, -GR¹⁰═CH—CH═CH—, —CH=GR¹⁰—CH═CH—, —CH═CH-GR¹⁰═CH—, —CH═CH—CH=GR¹⁰—, wherein G is either an sp²- or sp³-hybridized carbon or nitrogen atom; wherein R¹⁰ has the meaning set forth for R⁶; and a, b, c and d are each 0 or 1.

In certain embodiments of Formula 2, R¹ and R² form together for a fused 5- or 6-membered ring composed of one of the following bridging bivalent radicals (reading from R¹ to R²): -GR¹⁰—CH₂—CH₂—, —CH₂-GR¹⁰—CH₂—, —CH₂—CH₂-GR¹⁰—, —CH=GR¹⁰—CH═CH—, —CH═CH-GR¹⁰═CH—.

In a preferred embodiment of Formula 2, a is 1. In another preferred embodiment of Formula 2, D is selected from the group consisting of —OC(O)(CH₂)₃CH₃, —OC(O)CH₂C(CH₃)₃, —OC(O)(CH₂)₂-cyclopentyl and —OC(O)(CH₂)₂-cyclopropyl. In yet another embodiment of Formula 2, R¹⁰ is —OCH₃.

In still other embodiments, the compound of the invention is represented by the Formula 3, 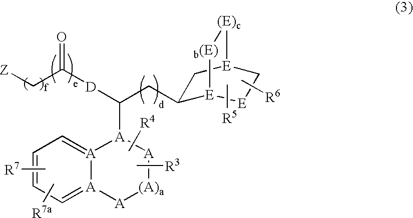
or a pharmaceutically acceptable salt thereof, wherein A and E are each, independently, an sp²- or sp³-hybridized carbon or nitrogen atom; D is selected from the group consisting of —CH₂CHCH—, —CH₂CHCHCH₂—, —O—, —[(CH₂)₁₋₆]—, —O—(CH₂)₁₋₆—, —O—(CH₂)₁₋₆—N(R⁹)—, —(CH₂)₁₋₆—N(R⁹)—, N(R⁹)—, wherein R⁹ is selected from the group consisting of —H, —C₁₋₄-alkyl, N-methyl-piperidinyl, morpholinyl, hydroxyphenyl, phenyl, piperazinyl, cyclopropyl, cyclopentyl, cyclohexyl, pyridinyl, 5H-tetrazolyl, triazolyl or piperidinyl; R³, R⁴, R⁵, R⁶, R⁷ and R^{7a} are each, independently, —H, —OH, —(CH₂)₀₋₆Y, —O—(CH₂)₀₋₆Y, wherein Y is selected from —H, —OH, —CN, halo, —CHCH₂, —CH₂CHCH₂, —NO₂, morpholinyl, hydroxyphenyl, phenyl, piperazinyl, cyclopropyl, cyclopentyl, cyclohexyl, pyridinyl, 5H-tetrazolyl, triazolyl, piperidinyl, alkylcarbonyl, alkylthiocarbonyl, alkoxycarbonyl, aminocarbonyl, —CO₂H; —SO₃H; —SO₂H; —SO₂NH₂, —SSO₃H, —PO₃H₂; —NO₂, —SH, —OSO₃H, —OC(O)(OH), —O—, —S—, halomethyl, dihalomethyl, trihalomethyl, —SO₂N(X¹)X² or N(X¹)X², wherein X¹ and X² are each, independently, H, aryl, C₁-C₆-alkyl, esters thereof; salts thereof, and any combination thereof, Z is selected from the group consisting of —H, —OH, —(CH₂)₀₋₆Y, —O—(CH₂)₀₋₆Y, wherein Y is selected from —H, —OH, —CN, halo, —CHCH₂, —CH₂CHCH₂, —NO₂, morpholinyl, hydroxyphenyl, phenyl, piperazinyl, cyclopropyl, cyclopentyl, cyclohexyl, pyridinyl, 5H-tetrazolyl, triazolyl, piperidinyl, alkylcarbonyl, alkylthiocarbonyl, alkoxycarbonyl, aminocarbonyl, —CO₂H, —SO₃H; —SO₂H; —SO₂NH₂, —SSO₃H, —PO₃H₂; —NO₂, —SH, —OSO₃H, —OC(O)(OH), —O—, —S—, halomethyl, dihalomethyl, trihalomethyl, —SO₂N(X¹)X² or N(X¹)X², wherein X¹ and X² are each, independently, H, aryl, C₁-C₆-alkyl, esters thereof, salts thereof, and any combination thereof; R⁷ and R^{7a} can also form together for a fused 5- or 6-membered ring composed of one of the following bridging bivalent radicals: —O—CH₂—CH₂—, —CH₂—O—CH₂—, —CH₂—CH₂—O—, —CH₂—CH₂—CH₂—, —O—CH₂—O—, —CH₂—CH₂—CH₂—CH₂—, a, b, c, d and e are each 0 or 1 and f is 0,1, 2, 3, 4, 5 or 6.

In certain embodiments of Formula 3, D is selected from the group consisting of O, N, or C₁₋₃-alkyl. In another embodiment of Formula 4, Z is selected from the group consisting of —H, cyclohexyl, cyclopentyl, phenyl, —CO₂H or piperidinyl. In another embodiment, R⁶ is selected from the group consisting of —CHCH₂, or (CH₂)₁₋₃Y, wherein Y is selected from the group consisting of —H, —OH, Br or CO₂H. In still another embodiment, R⁷ and R^{7a} are each, independently, selected from the group consisting of —H, —OCH₃, —NH₂ or —Br. In still another embodiment, R⁷ and R^{7a} form together for a fused 5-membered ring composed of the following bridging bivalent radical: —O—CH₂—O—. In another embodiment, f is 0-3. In another embodiment, D is —O—, e is 1, and f is 1 or 2. In another embodiment, Z is selected from the group consisting of H, —COOH, cyclopropyl, cyclopentyl, n-butyl and t-butyl. In another embodiment, R⁷ is —OCH₃, R^{7a} is H, and R³ and R⁴ are H. In another embodiment, R⁵ is H and R⁶ is —CHCH₂ or —CH₂CH₃.

In further embodiments, the compound of the invention is represented by the Formula 4, 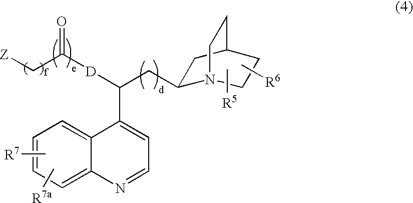
or a pharmaceutically acceptable salt thereof, wherein D is selected from the group consisting of —O—, —(CH₂)₁₋₆—, —N(R⁹)—, wherein R⁹ is selected from the group consisting of —H, —C₁₋₄-alkyl, N-methyl-piperidinyl, morpholinyl, hydroxyphenyl, phenyl, piperazinyl, cyclopropyl, cyclopentyl, cyclohexyl, pyridinyl, 5H-tetrazolyl, triazolyl, or piperidinyl; R⁵, R⁶, R⁷ and R^{7a} are each, independently, —H, —OH, —(CH₂)₀₋₆Y, —O—(CH₂)₀₋₆Y, wherein Y is selected from —H, —OH, —CN, halo, —CHCH₂, —CH₂CHCH₂, —NO₂, morpholinyl, hydroxyphenyl, phenyl, piperazinyl, cyclopropyl, cyclopentyl, cyclohexyl, pyridinyl, 5H-tetrazolyl, triazolyl, piperidinyl, alkylcarbonyl, alkylthiocarbonyl, alkoxycarbonyl, aminocarbonyl, —CO₂H; —SO₃H; —SO₂H; —SO₂NH₂, —SSO₃H, —PO₃H₂; —NO₂, —SH, —OSO₃H, —OC(O)(OH), —O—, —S—, halomethyl, dihalomethyl, trihalomethyl, —SO₂N(X¹)X² or N(X¹)X², wherein X¹ and X² are each, independently, H, aryl, C₁-C₆-alkyl, esters thereof; salts thereof, and any combination thereof; Z is selected from the group consisting of —H, —OH, —(CH₂)₀₋₆Y, —O—(CH₂)₀₋₆Y, wherein Y is selected from —H, —OH, —CN, halo, —CHCH₂, —CH₂CHCH₂, —NO₂, morpholinyl, hydroxyphenyl, phenyl, piperazinyl, cyclopropyl, cyclopentyl, cyclohexyl, pyridinyl, 5H-tetrazolyl, triazolyl, piperidinyl, alkylcarbonyl, alkylthiocarbonyl, alkoxycarbonyl, aminocarbonyl, —CO₂H, —SO₃H; —SO₂H; —SO₂NH₂, —SSO₃H, —PO₃H₂; —NO₂, —SH, —OSO₃H, —OC(O)(OH), —O—, —S—, halomethyl, dihalomethyl, trihalomethyl, —SO₂N(X¹)X² or N(X¹)X², wherein X¹ and X² are each, independently, H, aryl, C₁-C₆-alkyl, esters thereof; salts thereof, and any combination thereof; R⁷ and R^{7a} can also form together for a fused 5- or 6-membered ring composed of one of the following bridging bivalent radicals: —O—CH₂—CH₂—, —CH₂—O—CH₂—, —CH₂—CH₂—O—, —CH₂—CH₂—CH₂—, —O—CH₂—O—, —CH₂—CH₂—CH₂—CH₂—, d and e are each, independently, 0 or 1; f is 0, 1, 2, 3, 4, 5 or 6.

In cerain embodiments, D is selected from the group consisting of O, N, or C₁₋₃-alkyl. In another embodiment, Z is selected from the group consisting of —H, cyclohexyl, cyclopentyl, phenyl, —CO₂H or piperidinyl. In other embodiments, R⁶ is selected from the group consisting of —CHCH₂, or (CH₂)₁₋₃Y, wherein Y is selected from the group consisting of —H, —OH, Br or CO₂H. In other embodiments, R⁷ and R^{7a} are each, independently, selected from the group consisting of —H, —OCH₃, —NH₂ or —Br. In otherr embodiment, R⁷ and R^{7a} form together for a fused 5-membered ring composed of the following bridging bivalent radicals: —O—CH₂—O—. In other embodiments, f is 0-3. In other embodiments, D is —O—, e is 1, and f is 1 or 2. In other embodiments, Z is selected from the group consisting of H, —COOH, cyclopropyl, cyclopentyl, n-butyl and t-butyl. In otherr embodiments, R⁷ is —OCH₃, R^{7a} is H, and R³ and R⁴ are H. In another embodiment, R⁵ is H and R⁶ is —CHCH₂ or —CH₂CH₃.

In other embodiments, the compound is represented by the Formula 5, 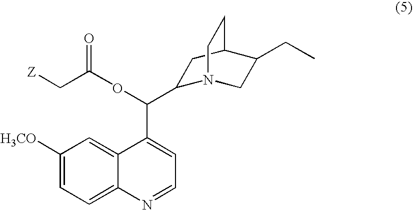
or a pharmaceutically acceptable salt thereof, wherein Z is selected from the group consisting of —H, —OH, —(CH₂)₀₋₆Y, —O—(CH₂)₀₋₆Y, wherein Y is selected from —H, C₁-C₆-alkyl, —OH, —CN, halo, —CHCH₂, —CH₂CHCH₂, —NO₂, morpholinyl, hydroxyphenyl, phenyl, piperazinyl, cyclopropyl, cyclopentyl, cyclohexyl, pyridinyl, 5H-tetrazolyl, triazolyl, piperidinyl, alkylcarbonyl, alkylthiocarbonyl, alkoxycarbonyl, aminocarbonyl, —CO₂H, —SO₃H; —SO₂H; —SO₂NH₂, —SSO₃H, —PO₃H₂; —NO₂, —SH, —OSO₃H, —OC(O)(OH), —O—, —S—, halomethyl, dihalomethyl, trihalomethyl, —SO₂N(X¹)X² or N(X¹)X², wherein X¹ and X² are each, independently, H, aryl, C₁-C₆-alkyl, esters thereof; salts thereof, and any combination thereof. In one embodiment, Z is selected from the group consisting of H, —COOH, cyclopropyl, cyclopentyl, CH₂CH₂CH₃, n-butyl and t-butyl.

In another embodiment, the compound is selected from the group consisting of 3-cyclopentyl-propionic acid quinolin-4-yl-(5-vinyl-1-aza-bicyclo[2.2.2]oct-2-yl)-methyl ester; cyclopropanecarboxylic acid quinolin-4-yl-(5-vinyl-1-aza-bicyclo[2.2.2]oct-2-yl)-methyl ester; succinic acid mono-[quinolin-4-yl-(5-vinyl-1-aza-bicyclo[2.2.2]oct-2-yl)-methyl]ester; 3-cyclopentyl-propionic acid (6-methoxy-quinolin-4-yl)-(5-vinyl-1-aza-bicyclo[2.2.2]oct-2-yl)-methyl ester; 2-[(3-cyclopentyl-propionyloxy)-(6-methoxy-quinolin-4-yl)-methyl]-5-vinyl-1-azonia-bicyclo[2.2.2]octane dihydrochloride; 3-cyclopentyl-propionic acid (5-ethyl-1-aza-bicyclo[2.2.2]oct-2-yl)-(6-methoxy-quinolin-4-yl)-methyl ester; 2-[(3-cyclopentyl-propionyloxy)-(6-methoxy-quinolin-4-yl)-methyl]-5-ethyl-1-azonia-bicyclo[2.2.2]octane dihydrochloride; pentanoic acid (6-methoxy-quinolin-4-yl)-(5-vinyl-1-aza-bicyclo[2.2.2]oct-2-yl; 2-[(6-methoxy-quinolin-4-yl)-pentanoyloxy-methyl]-5-vinyl-1-azonia-bicyclo[2.2.2]octane dihydrochloride; pentanoic acid (5-ethyl-1-aza-bicyclo[2.2.2]oct-2-yl)-(6-methoxy-quinolin-4-yl)-methyl ester; 5-ethyl-2-[(6-methoxy-quinolin-4-yl)-pentanoyloxy-methyl]-1-azonia-bicyclo[2.2.2]octane dihydrochloride; 3,3-dimethyl-butyric acid (6-methoxy-quinolin-4-yl)-(5-vinyl-1-aza-bicyclo[2.2.2]oct-2-yl)-methyl ester; 2-[(3,3-dimethyl-butyryloxy)-(6-methoxy-quinolin-4-yl)-methyl]-5-vinyl-1-azonia-bicyclo[2.2.2]octane dihydrochloride; 3,3-dimethyl-butyric acid (5-ethyl-1-aza-bicyclo[2.2.2]oct-2-yl)-(6-methoxy-quinolin-4-yl)-methyl ester and 2-[(3,3-dimethyl-butyryloxy)-(6-methoxy-quinolin-4-yl)-methyl]-5-ethyl-1-azonia-bicyclo[2.2.2]octane dihydrochloride.

In another aspect of the invention, contacting the cells with an effective amount of the compound inhibits the activity of a gated ion channel. In certain embodiments, the gated ion channel is comprised of at least one subunit selected from the group consisting of a member of the DEG/ENaC, P2X, and TRPV gene superfamilies. In other embodiments, the gated ion channel is comprised of at least one subunit selected from the group consisting of αENaC, βENaC, γENaC, δENaC, ASIC1a, ASIC1b, ASIC2a, ASIC2b, ASIC3, ASIC4, BLINaC, hINaC, P2X₁, P2X₂, P2X₃, P2X₄, P2X₅, P2X₆, P2X₇, TRPV1, TRPV2, TRPV3, TRPV4, TRPV5, and TRPV6. In certain embodiments, the gated ion channel is homomultimeric. In other embodiments, the gated ion channel is heteromultimeric. In certain embodiments, the DEG/ENaC gated ion channel is comprised of at least one subunit selected from the group consisting of αENaC, βENaC, γENaC, δENaC, BLINaC, hINaC, ASIC1a, ASIC1b, ASIC2a, ASIC2b, ASIC3, and ASIC4. In other embodiments, the DEG/ENaC gated ion channel is comprised of at least one subunit selected from the group consisting of ASIC1a, ASIC1b, ASIC2a, ASIC2b, ASIC3, and ASIC4. In one embodiment, the gated ion channel comprises ASIC1a. In another embodiment, the gated ion channel comprises ASIC3. In other embodiments, the P2X gated ion channel comprises at least one subunit selected from the group consisting of P2X₁, P2X₂, P2X₃, P2X₄, P2X₅, P2X₆, and P2X₇. In another embodiment, the TRPV gated ion channel comprises at least one subunit selected from the group TRPV1, TRPV2, TRPV3, TRPV4, TRPV5, and TRPV6. In another embodiment, the heteromultimeric gated ion channels include the following combinations of gated ion channels: αENaC, βENaC and γENaC; αENaC, βENaC and δENaC; ASIC1a and ASIC2a; ASIC1a and ASIC2b; ASIC1a and ASIC3; ASIC1b and ASIC3; ASIC2a and ASIC2b; ASIC2a and ASIC3; ASIC2b and ASIC3; ASIC1a, ASIC2a and ASIC3; P2X1 and P2X2; P2X1 and P2X5; P2X2 and P2X3; P2X2 and P2X6; P2X4 and P2X6; TRPV1 and TRPV2; TRPV5 and TRPV6; and TRPV1 and TRPV4. In another embodiment, heteromultimeric gated ion channels include the following combinations of gated ion channels: ASIC1a and ASIC2a; ASIC1a and ASIC2b; ASIC1a and ASIC3; ASIC2a and ASIC2b; ASIC1b and ASIC3; ASIC2a and ASIC3; and ASIC3 and ASIC2b.

In another aspect, the invention provides a method to modulate the activity of the gated ion channel in a subject in need thereof. In some embodiments, the gated ion channel acitivity is associated with pain. In other embodiments, the activity of the gated ion channel is associated with an inflammatory disorder. In other embodiments the gated ion channel activity is associated with ischemia. In other embodiments, the activity of the gated ion channel is associated with a neurological disorder.

In certain embodiments, the pain is selected from the group consisting of cutaneous pain, somatic pain, visceral pain and neuropathic pain. In another embodiment, the pain is acute pain or chronic pain.

In some embodiments, the cutaneous pain is associated with injury, trauma, a cut, a laceration, a puncture, a burn, a surgical incision, an infection or acute inflammation.

In some embodiments, the somatic pain is associated with an injury, disease or disorder of the musculoskeletal and connective tissue system. In certain embodiments, injury, disease or disorder of the musculoskeletal and connective tissue system is selected from the group consisting of sprains, broken bones, arthritis, psoriasis, eczema, and ischemic heart disease.

In some embodiments, the visceral pain is associated with an injury, disease or disorder of the circulatory system, the respiratory system, the gastrointestinal system, or the genitourinary system. In certain embodiments, the disease or disorder of the circulatory system is selected from the group consisting of ischaemic heart disease, angina, acute myocardial infarction, cardiac arrhythmia, phlebitis, intermittent claudication, varicose veins, myocardial infarction, hypertension and hypotension, rheumatic fever, pulmonary embolism, cerebrovascular diseases (e.g., stroke), atherosclerosis, peripheral vascular disease (e.g., intermittent claudication), thrombophlebitis and haemorrhoids. In certain embodiments, the disease or disorder of the respiratory system is selected from the group consisting of asthma, respiratory infection, chronic bronchitis, chronic obstructive pulmonary diseases, pulmonary oedema, cough, sinusitis, pharyngitis, and emphysema. In certain embodiments, the disease or disorder of the gastrointestinal system is selected from the group consisting of gastritis, duodenitis, irritable bowel syndrome, colitis, Crohn's disease, gastrointestinal reflux disease, ulcers, ulcerative colitis, paralytic ileus, oesophagitis, gastroenteritis, gastro-oesophageal reflux disease, hepatitis, cirrhosis and diverticulitis. In certain embodiments, the disease or disorder of the genitourinary system is selected from the group consisting of cystitis, urinary tract infections, glomuerulonephritis, polycystic kidney disease, kidney stones, nephritic syndrome, nephritis (e.g., interstitial nephritis), neurogenic bladder, prostatitis, endometriosis, and cancers of the genitourinary system. In other embodiments, the somatic pain is selected from the group consisting of arthralgia, myalgia, chronic lower back pain, phantom limb pain, cancer-associated pain, dental pain, fibromyalgia, idiopathic pain disorder, chronic non-specific pain, chronic pelvic pain, post-operative pain, and referred pain. In other embodiments, the neuropathic pain is associated with an injury, disease or disorder of the nervous system. In another embodiment, the injury, disease or disorder of the nervous system is selected from the group consisting of neuralgia, neuropathy, headache, psychogenic pain, chronic cephalic pain and spinal cord injury.

In other embodiments, the inflammatory disorder is selected from an inflammatory disorder of the musculoskeletal and connective tissue system, the respiratory system, the circulatory system, the genitourinary system the gastrointestinal system or the nervous system. In certain embodiments, the inflammatory disorder of the musculoskeletal and connective tissue system is selected from the group consisting of arthritis (e.g., inflammatory polyarthropathies (e.g., rheumatoid arthritis, juvenile arthritis, polyarthritis, gout), osteoarthritis, rheumatoid arthritis, and spondyloarthropathies), psoriasis, myocitis, dermatitis and eczema. In certain embodiments, the inflammatory disorder of the respiratory system is selected from the group consisting of asthma, bronchitis, sinusitis, pharyngitis, laryngitis, tracheitis, rhinitis, cystic fibrosis, respiratory infection and acute respiratory distress syndrome. In certain embodiments, the inflammatory disorder of the circulatory system is selected from the group consisting of vasculitis, haematuria syndrome, artherosclerosis, arteritis, phlebitis, carditis and coronary heart disease. In certain embodiments, the inflammatory disorder of the gastrointestinal system is selected from the group consisting of inflammatory bowel disorder, ulcerative colitis, Crohn's disease, diverticulitis, viral infection, bacterial infection, peptic ulcer, chronic hepatitis, gingivitis, periodentitis, stomatitis, gastritis and gastrointestinal reflux disease. In certain embodiments, the inflammatory disorder of the genitourinary system is selected from the group consisting of cystitis, polycystic kidney disease, nephritic syndrome, urinary tract infection, cystinosis, prostatitis, salpingitis, endometriosis, allergy (including allergic rhinitis/sinusitis, skin allergies (urticaria/hives, angioedema, atopic dermatitis), food allergies, drug allergies, insect allergies, and rare allergic disorders such as mastocytosis), autoimmune conditions (e.g., systemic lupus erythematosus, dermatomyositis, polymyositis, inflammatory neuropathies, Guillain Barré syndrome, inflammatory polyneuropathies, vasculitis, Wegener's granulomatosus and polyarteritis nodosa), and genitourinary cancer.

In other embodiments, the neurological disorder is selected from the group consisting of schizophrenia, bipolar disorder, depression, Alzheimer's disease, epilepsy, multiple sclerosis, amyotrophic lateral sclerosis, stroke, addiction, cerebral ischemia, neuropathy, retinal pigment degeneration, glaucoma, cardiac arrhythmia, shingles, Huntington's chorea, diseases of the eye (e.g., claucoma, chorioretinal inflammation, chroidal degeneration, retinal vascular occlusions, peripheral retinal degeneration, retinopathies (e.g. diabetic retinopathy, atherosclerotic retinopathy), and optic neuropathies), mental and behavioral disorders (e.g., panic attacks and phobias) and Parkinson disease.

In another aspect, the invention provides a method of treating pain in a subject in need thereof, comprising administering to the subject an effective amount of a compound of Formula 1, Formula 2, Formula 3, Formula 4 and Formula 5. In some embodiments, the compound is selected from the group consisting of 3-cyclopentyl-propionic acid quinolin-4-yl-(5-vinyl-1-aza-bicyclo[2.2.2]oct-2-yl)-methyl ester; cyclopropanecarboxylic acid quinolin-4-yl-(5-vinyl-1-aza-bicyclo[2.2.2]oct-2-yl)-methyl ester; succinic acid mono-[quinolin-4-yl-(5-vinyl-1-aza-bicyclo[2.2.2]oct-2-yl)-methyl]ester; 3-cyclopentyl-propionic acid (6-methoxy-quinolin-4-yl)-(5-vinyl-1-aza-bicyclo[2.2.2]oct-2-yl)-methyl ester; 2-[(3-cyclopentyl-propionyloxy)-(6-methoxy-quinolin-4-yl)-methyl]-5-vinyl-1-azonia-bicyclo[2.2.2]octane dihydrochloride; 3-cyclopentyl-propionic acid (5-ethyl-1-aza-bicyclo[2.2.2]oct-2-yl)-(6-methoxy-quinolin-4-yl)-methyl ester; 2-[(3-cyclopentyl-propionyloxy)-(6-methoxy-quinolin-4-yl)-methyl]-5-ethyl-1-azonia-bicyclo[2.2.2]octane dihydrochloride; pentanoic acid (6-methoxy-quinolin-4-yl)-(5-vinyl-1-aza-bicyclo[2.2.2]oct-2-yl; 2-[(6-methoxy-quinolin-4-yl)-pentanoyloxy-methyl]-5-vinyl-1-azonia-bicyclo[2.2.2]octane dihydrochloride; pentanoic acid (5-ethyl-1-aza-bicyclo[2.2.2]oct-2-yl)-(6-methoxy-quinolin-4-yl)-methyl ester; 5-ethyl-2-[(6-methoxy-quinolin-4-yl)-pentanoyloxy-methyl]-1-azonia-bicyclo[2.2.2]octane dihydrochloride; 3,3-dimethyl-butyric acid (6-methoxy-quinolin-4-yl)-(5-vinyl-1-aza-bicyclo[2.2.2]oct-2-yl)-methyl ester; 2-[(3,3-dimethyl-butyryloxy)-(6-methoxy-quinolin-4-yl)-methyl]-5-vinyl-1-azonia-bicyclo[2.2.2]octane dihydrochloride; 3,3-dimethyl-butyric acid (5-ethyl-1-aza-bicyclo[2.2.2]oct-2-yl)-(6-methoxy-quinolin-4-yl)-methyl ester and 2-[(3,3-dimethyl-butyryloxy)-(6-methoxy-quinolin-4-yl)-methyl]-5-ethyl-1-azonia-bicyclo[2.2.2]octane dihydrochloride.

In some embodiments, the subject is a mammal. In certain embodiments, the mammal is a human. In another embodiment, the pain is selected from the group consisting of cutaneous pain, somatic pain, visceral pain and neuropathic pain. In other embodiments, the pain is acute pain or chronic pain.

In another aspect, the invention provides a method of treating an inflammatory disorder in a subject in need thereof, comprising administering to the subject an effective amount of a compound of Formula 1, Formula 2, Formula 3, Formula 4, and Formula 5. In one embodiment, the compound is selected from the group consisting of 3-cyclopentyl-propionic acid quinolin-4-yl-(5-vinyl-1-aza-bicyclo[2.2.2]oct-2-yl)-methyl ester; cyclopropanecarboxylic acid quinolin-4-yl-(5-vinyl-1-aza-bicyclo[2.2.2]oct-2-yl)-methyl ester; succinic acid mono-[quinolin-4-yl-(5-vinyl-1-aza-bicyclo[2.2.2]oct-2-yl)-methyl]ester; 3-cyclopentyl-propionic acid (6-methoxy-quinolin-4-yl)-(5-vinyl-1-aza-bicyclo[2.2.2]oct-2-yl)-methyl ester; 2-[(3-cyclopentyl-propionyloxy)-(6-methoxy-quinolin-4-yl)-methyl]-5-vinyl-1-azonia-bicyclo[2.2.2]octane dihydrochloride; 3-cyclopentyl-propionic acid (5-ethyl-1-aza-bicyclo[2.2.2]oct-2-yl)-(6-methoxy-quinolin-4-yl)-methyl ester; 2-[(3-cyclopentyl-propionyloxy)-(6-methoxy-quinolin-4-yl)-methyl]-5-ethyl-1-azonia-bicyclo[2.2.2]octane dihydrochloride; pentanoic acid (6-methoxy-quinolin-4-yl)-(5-vinyl-1-aza-bicyclo[2.2.2]oct-2-yl; 2-[(6-methoxy-quinolin-4-yl)-pentanoyloxy-methyl]-5-vinyl-1-azonia-bicyclo[2.2.2]octane dihydrochloride; pentanoic acid (5-ethyl-1-aza-bicyclo[2.2.2]oct-2-yl)-(6-methoxy-quinolin-4-yl)-methyl ester; 5-ethyl-2-[(6-methoxy-quinolin-4-yl)-pentanoyloxy-methyl]-1-azonia-bicyclo[2.2.2]octane dihydrochloride; 3,3-dimethyl-butyric acid (6-methoxy-quinolin-4-yl)-(5-vinyl-1-aza-bicyclo[2.2.2]oct-2-yl)-methyl ester; 2-[(3,3-dimethyl-butyryloxy)-(6-methoxy-quinolin-4-yl)-methyl]-5-vinyl-1-azonia-bicyclo[2.2.2]octane dihydrochloride; 3,3-dimethyl-butyric acid (5-ethyl-1-aza-bicyclo[2.2.2]oct-2-yl)-(6-methoxy-quinolin-4-yl)-methyl ester and 2-[(3,3-dimethyl-butyryloxy)-(6-methoxy-quinolin-4-yl)-methyl]-5-ethyl-1-azonia-bicyclo[2.2.2]octane dihydrochloride.

In some embodiments, the subject is a mammal. In certain embodiments, the mammal is a human. In certain embodiments, the inflammatory disorder is inflammatory disorder of the musculoskeletal and connective tissue system, the respiratory system, the circulatory system, the genitourinary system, the gastrointestinal system or the nervous system.

In another aspect, the invention provides a method of treating a neurological disorder in a subject in need thereof, comprising administering an effective amount of a compound of Formula 1, Formula 2, Formula 3, Formula 4 and Formula 5. In one embodiment, the compound is selected from the group consisting of 3-cyclopentyl-propionic acid quinolin-4-yl-(5-vinyl-1-aza-bicyclo[2.2.2]oct-2-yl)-methyl ester; cyclopropanecarboxylic acid quinolin-4-yl-(5-vinyl-1-aza-bicyclo[2.2.2]oct-2-yl)-methyl ester; succinic acid mono-[quinolin-4-yl-(5-vinyl-1-aza-bicyclo[2.2.2]oct-2-yl)-methyl]ester; 3-cyclopentyl-propionic acid (6-methoxy-quinolin-4-yl)-(5-vinyl-1-aza-bicyclo[2.2.2]oct-2-yl)-methyl ester; 2-[(3-cyclopentyl-propionyloxy)-(6-methoxy-quinolin-4-yl)-methyl]-5-vinyl-1-azonia-bicyclo[2.2.2]octane dihydrochloride; 3-cyclopentyl-propionic acid (5-ethyl-1-aza-bicyclo[2.2.2]oct-2-yl)-(6-methoxy-quinolin-4-yl)-methyl ester; 2-[(3-cyclopentyl-propionyloxy)-(6-methoxy-quinolin-4-yl)-methyl]-5-ethyl-1-azonia-bicyclo[2.2.2]octane dihydrochloride; pentanoic acid (6-methoxy-quinolin-4-yl)-(5-vinyl-1-aza-bicyclo[2.2.2]oct-2-yl; 2-[(6-methoxy-quinolin-4-yl)-pentanoyloxy-methyl]-5-vinyl-1-azonia-bicyclo[2.2.2]octane dihydrochloride; pentanoic acid (5-ethyl-1-aza-bicyclo[2.2.2]oct-2-yl)-(6-methoxy-quinolin-4-yl)-methyl ester; 5-ethyl-2-[(6-methoxy-quinolin-4-yl)-pentanoyloxy-methyl]-1-azonia-bicyclo[2.2.2]octane dihydrochloride; 3,3-dimethyl-butyric acid (6-methoxy-quinolin-4-yl)-(5-vinyl-1-aza-bicyclo[2.2.2]oct-2-yl)-methyl ester; 2-[(3,3-dimethyl-butyryloxy)-(6-methoxy-quinolin-4-yl)-methyl]-5-vinyl-1-azonia-bicyclo[2.2.2]octane dihydrochloride; 3,3-dimethyl-butyric acid (5-ethyl-1-aza-bicyclo[2.2.2]oct-2-yl)-(6-methoxy-quinolin-4-yl)-methyl ester and 2-[(3,3-dimethyl-butyryloxy)-(6-methoxy-quinolin-4-yl)-methyl]-5-ethyl-1-azonia-bicyclo[2.2.2]octane dihydrochloride.

In some embodiments, the subject is a mammal. In certain embodiments, the mammal is a human. In certain embodiments, the neurological disorder is selected from the group consisting of schizophrenia, bipolar disorder, depression, Alzheimer's disease, epilepsy, multiple sclerosis, amyotrophic lateral sclerosis, stroke, addiction, cerebral ischemia, neuropathy, retinal pigment degeneration, glaucoma, cardiac arrhythmia, Huntington's chorea, and Parkinson disease.

In still other embodiments of the invention, the methods of the invention further comprise administering an adjuvant composition. In some embodiments, the adjuvant composition is selected from the group consisting of opioid analgesics, non-opioid analgesics, local anesthetics, corticosteroids, non-steroidal anti-inflammatory drugs, non-selective COX inhibitors, non-selective COX2 inhibitors, selective COX2 inhibitors, antiepileptics, barbiturates, antidepressants, marijuana, and topical analgesics.

In another aspect, the invention provides a pharmaceutical composition comprising a compound of Formula 1, Formula 2, Formula 3, Formula 4, Formula 5.

### BRIEF DESCRIPTION OF THE DRAWINGS

FIGS. 1A and 1B demonstrate results of Formalin tests described herein, demonstrating that compounds A and B both attenuated flinching behaviour compared with injection of vehicle.

FIG. 2 demonstrates concentration response relationships between Compound A and ASIC1a and ASIC3 that were acquired using the patch-clamp procedure described herein.

### DETAILED DESCRIPTION OF THE INVENTION

The present invention is based, at least in part, on the identification of compounds useful in modulation of the activity of gated ion channels. Gated ion channels are involved in receiving, conducting, and transmitting signals in a cell (e.g., an electrically excitable cell, for example, a neuronal or muscle cell). Gated ion channels can determine membrane excitability (the ability of, for example, a cell to respond to a stimulus and to convert it into a sensory impulse). Gated ion channels can also influence the resting potential of membranes, wave forms and frequencies of action potentials, and thresholds of excitation. Gated ion channels are typically expressed in electrically excitable cells, e.g., neuronal cells, and are multimeric; they may form homomultimeric (e.g., composed of one type of subunit), or heteromultimeric structures (e.g., composed of more than one type of subunit). Gated ion channels may also be found in nonexcitable cells (e.g., adipose cells or liver cells), where they may play a role in, for example, signal transduction.

As used herein, the terms “gated ion channel” or “gated channel” are used interchangeably and are intended to refer to a mammalian (e.g, rat, mouse, human)multimeric complex responsive to, for example, variations of voltage (e.g., membrane depolarization or hyperpolarization), temperature (e.g., higher or lower than 37° C.), pH (e.g., pH values higher or lower than 7.4), ligand concentration and/or mechanical stimulation. Examples of specific modulators include, but are not limited to, endogenous extracellular ligands such as anandamide, ATP, glutamate, cysteine, glycine, gamma-aminobutyric acid (GABA), histidine, serotonin (5HT), acetylcholine, epinephrine, norepinephrine, protons, ions, e.g., Na⁺, Ca⁺⁺, K⁺, Cl⁻, H⁺, Zn⁺, and/or peptides, e.g., Met-enkephaline, Leu-enkephaline, dynorphin, neurotrophins, and /or the RFamide related peptides, e.g., FMRFamide and/or FLRFamide; to endogenous intracellular ligands such as cyclic nucleotides (e.g. cyclicAMP, cyclicGMP), ATP, Ca⁺⁺ and/or G-proteins; to exogenous extracellular ligands or modulators such as α-amino-3-hydroxy-5-methyl-4-isolaxone propionate (AMPA), amiloride, capsaicin, capsazepine, epibatidine, cadmium, barium, gadolinium, guanidium, kainate, N-methyl-D-aspartate (NMDA). Gated ion channels also include complexes responsive to toxins, examples of which include but are not limited to, Agatoxin (e.g. α-agatoxin IVA, IVB, ω-agatoxin IVA, TK), Agitoxins (Agitoxin 2), Apamin, Argiotoxins, Batrachotoxins, Brevetoxins (e.g. Brevetoxin PbTx-2, PbTx-3, PbTx-9), Charybdotoxins, Chlorotoxins, Ciguatoxins, Conotoxins (e.g α-conotoxin GI, GIA, GII, IMI, MI, MII, SI, SIA, SII, and/or EI; δ-conotoxins, μ-conotoxin GIIIA, GIIIB, GIIIC and/or GS, ω-conotoxin GVIA, MVIIA MVIIC, MVIID, SVIA and/or SVIB), Dendrotoxins, Graminotoxins (GsMTx-4, ω-grammotoxin SIA), Grayanotoxins, Hanatoxins, Iberiotoxins, Imperatoxins, Jorotoxins, Kaliotoxins, Kurtoxins, Leiurotoxin 1, Pricotoxins, Psalmotoxins, (e.g., Psalmotoxin 1 (PcTx1)), Margatoxins, Noxiustoxins, Phrixotoxins, PLTX II, Saxitoxins, Stichodactyla Toxins, sea anemone toxins (e.g. APETx2 from Anthopleura elegantissima), Tetrodotoxins, Tityus toxin K-α, Scyllatoxins and/or tubocurarine.

Gated ion channels are generally homomeric or heteromeric complexes composed of subunits, comprising at least one subunit belonging to the DEG/ENaC, TRPV and/or P2X gene superfamilies. Non-limiting examples of the DEG/ENaC receptor gene superfamily include epithelial Na⁺ channels, e.g., αENaC, βENaC, γENaC, and/or δENaC, the mammalian degenerins (also referred to as MDEG, brain Na⁺ channels (BNaC, BNC) and the acid sensing ion channels (ASICs), e.g., ASIC1, ASIC1a, ASIC1b, ASIC2, ASIC2a, ASIC2b, ASIC3, and/or ASIC4. Non-limiting examples of the P2X receptor gene superfamily include P2X₁, P2X₂, P2X₃, P2X₄, P2X₅, P2X₆, and P2X₇. Non-limiting examples of the TRPV receptor gene superfamily include TRPV1 (also referred to as VR1), TRPV2 (also referred to as VRL-1), TRPV3 (also referred to as VRL-3), TRPV4 (also referred to as VRL-2), TRPV5 (also referred to as ECAC-1), and/or TRPV6 (also referred to as ECAC-2).

Non limiting examples of heteromultimeric gated ion channels include αENaC, βENaC and γENaC; αENaC, βENaC and δENaC; ASIC1a and ASIC2a; ASIC1a and ASIC2b; ASIC1a and ASIC3; ASIC1b and ASIC3; ASIC2a and ASIC2b; ASIC2a and ASIC3; ASIC2b and ASIC3; ASIC1a, ASIC2a and ASIC3; ASIC3 and P2X, e.g. P2X1, P2X2, P2X3, P2X4, P2X5, P2X6 and P2X7, preferably ASIC3 and P2X2; ASIC3 and P2X3; and ASIC3, P2X2 and P2X3 ASIC4 and at least one of ASIC1a, ASIC1b, ASIC2a, ASIC2b, and ASIC3; BLINaC (or hINaC) and at least one of ASIC1a, ASIC1b, ASIC2a, ASIC2b, ASIC3, and ASIC4; δENaC and ASIC, e.g ASIC1a, ASIC1b, ASIC2a, ASIC2b, ASIC3 and ASIC4; P2X1 and P2X2, P2X1 and P2X5, P2X2 and P2X3, P2X2 and P2X6, P2X4 and P2X6, TRPV1 and TRPV2, TRPV5 and TRPV6, TRPV1 and TRPV4.

I. Compounds of the Invention

The present invention provides compounds which modulate the activity of a gated ion channel. In some embodiments, the compounds of the invention modulate the activity of a gated ion channel comprised of at least one subunit belonging to the DEG/ENaC, TRPV and/or P2X gene superfamilies. In some embodiments, the compounds of the invention modulate the activity of the gated ion channel comprised of at least one subunit selected from the group consisting of αENaC, βENaC, γENaC, δENaC, ASIC1a, ASIC1b, ASIC2a, ASIC2b, ASIC3, ASIC4, BLINaC, MNaC, P2X₁, P2X₂, P2X₃, P2X₄, P2X₅, P2X₆, P2X₇, TRPV1, TRPV2, TRPV3, TRPV4, TRPV5, and TRPV6. In still other embodiments, the compounds of the invention modulate the activity of the DEG/ENaC gated ion channel comprised of at least one subunit selected from the group consisting of αENaC, βENaC, γENaC, δENaC, BLINaC, hINaC, ASIC1a, ASIC1b, ASIC2a, ASIC2b, ASIC3, and ASIC4. In certain embodiments, the compounds of the invention modulate the activity of the DEG/ENaC gated ion channel comprised of at least one subunit selected from the group consisting of ASIC1a, ASIC1b, ASIC2a, ASIC2b, ASIC3, and ASIC4. In certain embodiments, the compounds of the invention modulate the activity of the DEG/ENaC gated ion channel comprised of at least two subunits selected from the group consisting of ASIC1a, ASIC1b, ASIC2a, ASIC2b, ASIC3, and ASIC4. In yet other embodiments, the compounds of the invention modulate the activity of the DEG/ENaC gated ion channel comprised of at least three subunits selected from the group consisting of ASIC1a, ASIC1b, ASIC2a, ASIC2b, ASIC3, and ASIC4. In certain embodiments, the compounds of the invention modulate the activity of a gated ion channel comprised of ASIC, i.e., ASIC1a or ASIC1b. In certain embodiments, the compounds of the invention modulate the activity of a gated ion channel comprised of ASIC3. In certain embodiments, the compounds of the invention modulate the activity of a gated ion channel comprised of ASIC1a and ASIC2a,; ASIC1a and ASIC2a; ASIC1a and ASIC3; ASIC1b and ASIC3; ASIC2a and ASIC2b; ASIC2a and ASIC3; ASIC2b and ASIC3; ASIC1a and ASIC3; and ASIC1a, ASIC2a and ASIC3. In other embodiments, the compounds of the invention modulate the activity of the P2X gated ion channel comprised of at least one subunit selected from the group consisting of P2X₁, P2X₂, P2X₃, P2X₄, P2X₅, P2X₆, and P2X₇. In certain embodiments, the compounds of the invention modulate the activity of a gated ion channel comprised of P2X2, P2X3 or P2X4. In certain embodiments, the compounds of the invention modulate the activity of a gated ion channel comprised of P2X1 and P2X2, P2X1 and P2X5, P2X2 and P2X3, P2X2 and P2X6, and P2X4 and P2X6. In yet another aspect of the invention, the compounds of the invention modulate the activity of the TRPV gated ion channel comprised of at least one subunit selected from the group TRPV1, TRPV2, TRPV3, TRPV4, TRPV5, and TRPV6. In certain embodiments, the compounds of the invention modulate the activity of a gated ion channel comprised of TRPV1 or TRPV2. In certain embodiments, the compounds of the invention modulate the activity of a gated ion channel comprised of TRPV1 and TRPV2, TRPV1 and TRPV4, and TRPV5 and TRPV6.

In one apect, the compound that modulates the activity of a gated ion channel is of the Formula 1, 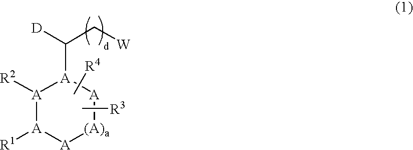
or a pharmaceutically acceptable salt thereof, wherein A is, independently, either an sp²- or sp³-hybridized carbon or nitrogen atom; D is selected from the group consisting of —H, —OH, halogen, —(CH₂)₀₋₆Y, —O—(CH₂)₀₋₆Y, wherein Y is selected from —H, —CN, —CO₂H, —SO₃H, —SO₂H, —PO₃H₂, —NO₂, —SSO₃H, halomethyl, dihalomethyl, trihalomethyl, N-methyl-piperidinyl, morpholinyl, hydroxyphenyl, phenyl, piperazinyl, cyclopropyl, cyclopentyl, cyclohexyl, pyridinyl, 5H-tetrazolyl, triazolyl or piperidinyl; R⁹(CH₂)₀₋₆COO—, —N(R⁹)(CH₂)₀₋₆COO(R⁹), —O(CH₂)₀₋₆(R⁹), —(CH₂)₁₋₆COO(R⁹), —(CH₂)₁₋₆N(R⁹)COO(R⁹), —(CH₂)₁₋₆N(R⁹)CO(R⁹), —(CH₂)₁₋₆CONH(R⁹), ═NOR⁹, wherein R⁹ is selected from the group consisting of —H, —C₁₋₄-alkyl, N-methyl-piperidinyl, morpholinyl, hydroxyphenyl, phenyl, piperazinyl, cyclopropyl, cyclopentyl, cyclohexyl, pyridinyl, 5H-tetrazolyl, triazolyl, or piperidinyl; —N(X¹)X², —SO₂N(X¹)X², wherein X¹ and X² are each, independently, H, aryl, or C₁-C₆-alkyl; salts thereof, esters thereof, and any combination thereof; W is selected from the group consisting of —H, —OH, halogen, —(CH₂)₀₋₆Y, —O—(CH₂)₀₋₆Y, wherein Y is selected from —H, —CN—CO₂H, —SO₃H, —SO₂H, —PO₃H₂, —NO₂, —SSO₃H, halomethyl, dihalomethyl, trihalomethyl, N-methyl-piperidinyl, morpholinyl, hydroxyphenyl, phenyl, piperazinyl, cyclopropyl, cyclopentyl, cyclohexyl, pyridinyl, 5H-tetrazolyl, triazolyl or piperidinyl; R⁹(CH₂)₀₋₆COO—, —N(R⁹)(CH₂)₀₋₆COO(R⁹), —O(CH₂)₀₋₆(R⁹), —(CH₂)₁₋₆COO(R⁹), —(CH₂)₁₋₆N(R⁹)COO(R⁹), —(CH₂)₁₋₆N(R⁹)CO(R⁹), —(CH₂)₁₋₆CONH(R⁹), ═NOR⁹, wherein R⁹ is selected from the group consisting of —H, —C₁₋₄-alkyl, N-methyl-piperidinyl, morpholinyl, hydroxyphenyl, phenyl, piperazinyl, cyclopropyl, cyclopentyl, cyclohexyl, pyridinyl, 5H-tetrazolyl, triazolyl, or piperidinyl; —N(X¹)X², —SO₂N(X¹)X², or —(CH₂)₁₋₆SO₂N(X¹)X², wherein X¹ and X² are each, independently, H, aryl, C₁-C₆-alkyl; salts thereof, esters thereof, and any combination thereof; and any combination thereof; or the formula 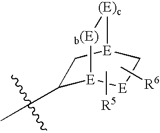
wherein E is, independently, either an sp²- or sp³-hybridized carbon or nitrogen atom; R⁵ and R⁶ are each, independently, —H, —OH, —(CH₂)₀₋₆Y, —O—(CH₂)₀₋₆Y, wherein Y is selected from —H, —OH, —CN, halo, —CHCH₂, —CH₂CHCH₂, —NO₂, morpholinyl, hydroxyphenyl, phenyl, piperazinyl, cyclopropyl, cyclopentyl, cyclohexyl, pyridinyl, 5H-tetrazolyl, triazolyl, piperidinyl, alkylcarbonyl, alkylthiocarbonyl, alkoxycarbonyl, aminocarbonyl, —CO₂H; —SO₃H; —SO₂H; —SO₂NH₂, —SSO₃H, —PO₃H₂; —NO₂, —SH, —OSO₃H, —OC(O)(OH), —O—, —S—, halomethyl, dihalomethyl, trihalomethyl, —SO₂N(X¹)X² or N(X¹)X², wherein X¹ and X² are each, independently, H, aryl, C₁-C₆-alkyl, esters thereof; salts thereof, and any combination thereof; R¹, R², R³, R⁴, are each, independently, selected from the group consisting of —H, —OH, halogen, —(CH₂)₀₋₆Y, —O—(CH₂)₀₋₆Y, wherein Y is selected from —H, —CN, —CO₂H, —SO₃H, —SO₂H, —PO₃H₂, —NO₂, —SSO₃H, halomethyl, dihalomethyl, trihalomethyl, N-methyl-piperidinyl, morpholinyl, hydroxyphenyl, phenyl, piperazinyl, cyclopropyl, cyclopentyl, cyclohexyl, pyridinyl, 5H-tetrazolyl, triazolyl or piperidinyl; R⁹(CH₂)₀₋₆COO—, —N(R⁹)(CH₂)₀₋₆COO(R⁹), —(CH₂)₁₋₆N(R⁹)CO(R⁹), —O(CH₂)₀₋₆(R⁹), —(CH₂)₁₋₆COO(R⁹), —(CH₂)₁₋₆N(R⁹)COO(R⁹), —(CH₂)₁₋₆N(R⁹)CO(R⁹), —(CH₂)₁₋₆CONH(R⁹), ═NOR⁹, wherein R⁹ is selected from the group consisting of —H, —C₁₋₄-alkyl, N-methyl-piperidinyl, morpholinyl, hydroxyphenyl, phenyl, piperazinyl, cyclopropyl, cyclopentyl, cyclohexyl, pyridinyl, 5H-tetrazolyl, triazolyl, or piperidinyl; —N(X¹)X², —SO₂N(X¹)X² wherein X¹ and X² are each, independently, H, aryl, C₁-C₆-alkyl; salts thereof, esters thereof, and any combination thereof; R¹ and R² can also form together for a fused 5- or 6-membered ring composed of one of the following bridging bivalent radicals (reading from R¹ to R²):
-GR¹⁰—CH₂—CH₂—
—CH₂-GR¹⁰—CH₂—
—CH₂—CH₂-GR¹⁰—
-GR¹⁰—CH₂—CH₂—CH₂—
—CH₂-GR¹⁰—CH₂—CH₂—
—CH₂—CH₂-GR¹⁰—CH₂—
—CH₂—CH₂—CH₂-GR¹⁰—
-GR¹⁰═CH—CH═CH—
—CH=GR¹⁰—CH═CH—
—CH═CH—GR¹⁰═CH—
—CH═CH—CH=GR¹⁰—

wherein G is either an sp²- or sp³-hybridized carbon or nitrogen atom; wherein R¹⁰ has the meaning set forth for R⁶; and a, b, c and d are each 0 or 1.

In a preferred embodiment of Formula 1, W is 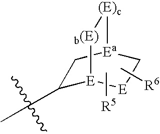
wherein E^{a} is N, E is C, R⁶ is H, and R⁵ is —CHCH₂ or —CH₂CH₃.

In another preferred emdodiment of Formula 1, R¹ and R² form together for a fused 5- or 6-membered ring composed of one of the following bridging bivalent radicals (reading from R¹ to R²):
-GR¹⁰—CH₂—CH₂—
—CH₂-GR¹⁰—CH₂—
—CH₂—CH₂-GR¹⁰—
—CH=GR¹⁰—CH═CH—
—CH═CH-GR¹⁰═CH—

In another preferred emdodiment of Formula 1, a is 1.

A preferred embodiment of Formula 1 is represented as Formula 2, 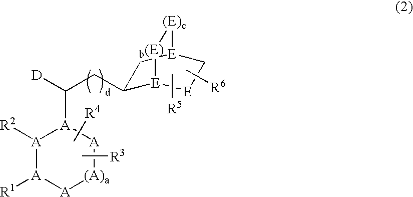
or a pharmaceutically acceptable salt thereof, wherein A and E are each, independently, an sp²- or sp³-hybridized carbon or nitrogen atom; D is selected from the group consisting of —H, —OH, halogen, —(CH₂)₀₋₆Y, —O—(CH₂)₀₋₆Y, wherein Y is selected from —H, —CN, —CO₂H, —SO₃H, —SO₂H, —PO₃H₂, —NO₂, —SSO₃H, halomethyl, dihalomethyl, trihalomethyl, N-methyl-piperidinyl, morpholinyl, hydroxyphenyl, phenyl, piperazinyl, cyclopropyl, cyclopentyl, cyclohexyl, pyridinyl, 5H-tetrazolyl, triazolyl or piperidinyl; R⁹(CH₂)₀₋₆COO—, —N(R⁹)(CH₂)₀₋₆COO(R⁹), —O(CH₂)₀₋₆(R⁹), —(CH₂)₁₋₆COO(R⁹), —(CH₂)₁₋₆N(R⁹)COO(R⁹), —(CH₂)₁₋₆N(R⁹)CO(R⁹), —(CH₂)₁₋₆CONH(R⁹), ═NOR⁹, wherein R⁹ is selected from the group consisting of —H, —C₁₋₄-alkyl, N-methyl-piperidinyl, morpholinyl, hydroxyphenyl, phenyl, piperazinyl, cyclopropyl, cyclopentyl, cyclohexyl, pyridinyl, 5H-tetrazolyl, triazolyl, or piperidinyl; —N(X¹)X², —SO₂N(X¹)X², wherein X¹ and X² are each, independently, H, aryl, C₁-C₆-alkyl; salts thereof, esters thereof, and any combination thereof; R¹, R², R³, R⁴, R⁵ and R⁶, are each, independently, —H, —OH, halo, C₁-C₆-alkyl, —O—C₁₋₆-alkyl, —CHCH₂, —CH₂CHCH₂, —(CH₂)₀₋₆Y, —O—(CH₂)₀₋₆Y, wherein Y is selected from —H, —CN, —CO₂H, —SO₃H, —SO₂H, —PO₃H₂, —NO₂, —SSO₃H, halomethyl, dihalomethyl, trihalomethyl, N-methyl-piperidinyl, morpholinyl, hydroxyphenyl, phenyl, piperazinyl, cyclopropyl, cyclopentyl, cyclohexyl, pyridinyl, 5H-tetrazolyl, triazolyl or piperidinyl; R⁹(CH₂)₀₋₆COO—, —N(R⁹)(CH₂)₀₋₆COO(R⁹), —O(CH₂)₀₋₆(R⁹), —(CH₂)₁₋₆COO(R⁹), —(CH₂)₁₋₆N(R)COO(R⁹), —(CH₂)₁₋₆N(R⁹)CO(R⁹), —(CH₂)₁₋₆CONH(R⁹), ═NOR⁹, wherein R⁹ is selected from the group consisting of —H, —C₁₋₄-alkyl, N-methyl-piperidinyl, morpholinyl, hydroxyphenyl, phenyl, piperazinyl, cyclopropyl, cyclopentyl, cyclohexyl, pyridinyl, 5H-tetrazolyl, triazolyl, or piperidinyl; —N(X¹)X², —SO₂N(X¹)X² wherein X¹ and X² are each, independently, H, aryl, C₁-C₆-alkyl; salts thereof, esters thereof, and any combination thereof; R¹ and R² can also form together for a fused 5- or 6-membered ring composed of one of the following bridging bivalent radicals (reading from R¹ to R²):
-GR¹⁰—CH₂—CH₂—
—CH₂-GR¹⁰—CH₂—
—CH₂—CH₂-GR¹⁰—
-GR¹⁰—CH₂—CH₂—CH₂—
—CH₂-GR¹⁰—CH₂—CH₂—
—CH₂—CH₂-GR¹⁰—CH₂—
—CH₂—CH₂—CH₂-GR¹⁰—
-GR¹⁰═CH—CH═CH—
—CH=GR¹⁰—CH═CH—
—CH═CH-GR¹⁰═CH—
—CH═CH—CH=GR¹⁰—

wherein G is either an sp²- or sp³-hybridized carbon or nitrogen atom; wherein R¹⁰ has the meaning set forth for R⁶; and a, b, c and d are each 0 or 1.

In a preferred embodiment of Formula 2, R¹ and R² form together for a fused 5- or 6-membered ring composed of one of the following bridging bivalent radicals (reading from R¹ to R²):
-GR¹⁰—CH₂—CH₂—
—CH₂-GR¹⁰—CH₂—
—CH₂—CH₂-GR¹⁰—
—CH=GR¹⁰—CH═CH—
—CH═CH-GR¹⁰═CH—

In another preferred embodiment of Formula 2, a is 1. In another preferred embodiment of Formula 2, D is selected from the group consisting of —OC(O)(CH₂)₃CH₃, —OC(O)CH₂C(CH₃)₃, —OC(O)(CH₂)₂-cyclopentyl and —OC(O)(CH₂)₂-cyclopropyl. In yet another preferred embodiment of Formula 2, R¹⁰ is —OCH₃.

Another preferred embodiment of Formula 1 is represented as Formula 3, 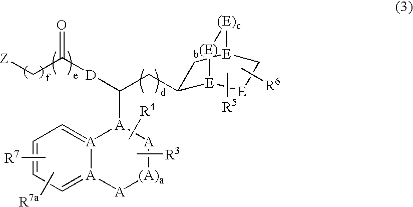
or a pharmaceutically acceptable salt thereof, wherein A and E are each, independently, an sp²- or sp³-hybridized carbon or nitrogen atom; D is selected from the group consisting of —CH₂CHCH—, —CH₂CHCHCH₂—, —O—, —[(CH₂)₁₋₆]—, —O—(CH₂)₁₋₆—, —O—(CH₂)₁₋₆—N(R⁹)—, —(CH₂)₁₋₆—N(R⁹)—, N(R⁹)-, wherein R⁹ is selected from the group consisting of —H, —C₁₋₄-alkyl, N-methyl-piperidinyl, morpholinyl, hydroxyphenyl, phenyl, piperazinyl, cyclopropyl, cyclopentyl, cyclohexyl, pyridinyl, 5H-tetrazolyl, triazolyl or piperidinyl; R³, R⁴, R⁵, R⁶, R⁷ and R^{7a} are each, independently, —H, —OH, —(CH₂)₀₋₆Y, —O—(CH₂)₀₋₆Y, wherein Y is selected from —H, —OH, —CN, halo, —CHCH₂, —CH₂CHCH₂, —NO₂, morpholinyl, hydroxyphenyl, phenyl, piperazinyl, cyclopropyl, cyclopentyl, cyclohexyl, pyridinyl, 5H-tetrazolyl, triazolyl, piperidinyl, alkylcarbonyl, alkylthiocarbonyl, alkoxycarbonyl, aminocarbonyl, —CO₂H; —SO₃H; —SO₂H; —SO₂NH₂, —SSO₃H, —PO₃H₂; —NO₂, —SH, —OSO₃H, —OC(O)(OH), —O—, —S—, halomethyl, dihalomethyl, trihalomethyl, —SO₂N(X¹)X² or N(X¹)X², wherein X¹ and X² are each, independently, H, aryl, C₁-C₆-alkyl, esters thereof; salts thereof, and any combination thereof; Z is selected from the group consisting of —H, —OH, —(CH₂)₀₋₆Y, —O—(CH₂)₀₋₆Y, wherein Y is selected from —H, —OH, —CN, halo, —CHCH₂, —CH₂CHCH₂, —NO₂, morpholinyl, hydroxyphenyl, phenyl, piperazinyl, cyclopropyl, cyclopentyl, cyclohexyl, pyridinyl, 5H-tetrazolyl, triazolyl, piperidinyl, alkylcarbonyl, alkylthiocarbonyl, alkoxycarbonyl, aminocarbonyl, —CO₂H, —SO₃H; —SO₂H; —SO₂NH₂, —SSO₃H, —PO₃H₂; —NO₂, —SH, —OSO₃H, —OC(O)(OH), —O—, —S—, halomethyl, dihalomethyl, trihalomethyl, —SO₂N(X¹)X² or N(X¹)X², wherein X¹ and X² are each, independently, H, aryl, C₁-C₆-alkyl, esters thereof; salts thereof, and any combination thereof; R⁷ and R^{7a} can also form together for a fused 5- or 6-membered ring composed of one of the following bridging bivalent radicals:
—O—CH₂—CH₂—
—CH₂—O—CH₂—
—CH₂—CH₂—O—
—CH₂—CH₂—CH₂—
—O—CH₂—O—
—CH₂—CH₂—CH₂—CH₂—
a, b, c, d and e are each 0 or 1; and f is 0, 1, 2, 3, 4, 5 or 6.

In a preferred embodiment of Formula 3, D is selected from the group consisting of O, N, or C₁₋₃-alkyl. In another preferred embodiment of Formula 3, Z is selected from the group consisting of —H, cyclohexyl, cyclopentyl, phenyl, —CO₂H or piperidinyl.

In another preferred embodiment of Formula 3, R⁶ is selected from the group consisting of —CHCH₂, or (CH₂)₁₋₃Y, wherein Y is selected from the group consisting of —H, —OH, Br or CO₂H. In another preferred embodiment of Formula 3, wherein R⁷ and R^{7a} are each, independently, selected from the group consisting of —H, —OCH₃, —NH₂ or —Br. In still another preferred embodiment of Formula 3, R⁷ and R^{7a} form together for a fused 5-membered ring composed of the following bridging bivalent radical: —O—CH₂—O—. In another preferred embodiment of Formula 3, f is 0-3. In another preferred embodiment of Formula 3, D is —O—, e is 1, and f is 1 or 2. In yet another preferred embodiment of Formula 3, Z is selected from the group consisting of H, —COOH, cyclopropyl, cyclopentyl, n-butyl and t-butyl. In still another preferred embodiment of Formula 3, R⁷ is —OCH₃, R^{7a} is H, and R³ and R⁴ are H. In another preferred embodiment of Formula 3, R⁵ is H and R⁶ is —CHCH₂ or —CH₂CH₃.

Another preferred embodiment of Formula 1 is represented as Formula 4, 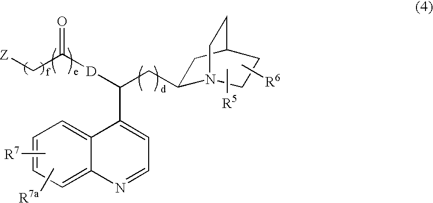
or a pharmaceutically acceptable salt thereof, wherein D is selected from the group consisting of —O—, —(CH₂)₁₋₆—, —N(R⁹)—, wherein R⁹ is selected from the group consisting of —H, —C₁₋₄-alkyl, N-methyl-piperidinyl, morpholinyl, hydroxyphenyl, phenyl, piperazinyl, cyclopropyl, cyclopentyl, cyclohexyl, pyridinyl, 5H-tetrazolyl, triazolyl, or piperidinyl; R⁵, R⁶, R⁷ and R^{7a} are each, independently, —H, —OH, —(CH₂)₀₋₆Y, —O—(CH₂)₀₋₆Y, wherein Y is selected from —H, —OH, —CN, halo, —CHCH₂, —CH₂CHCH₂, —NO₂, morpholinyl, hydroxyphenyl, phenyl, piperazinyl, cyclopropyl, cyclopentyl, cyclohexyl, pyridinyl, 5H-tetrazolyl, triazolyl, piperidinyl, alkylcarbonyl, alkylthiocarbonyl, alkoxycarbonyl, aminocarbonyl, —CO₂H; —SO₃H; —SO₂H; —SO₂NH₂, —SSO₃H, —PO₃H₂; —NO₂, —SH, —OSO₃H, —OC(O)(OH), —O—, —S—, halomethyl, dihalomethyl, trihalomethyl, —SO₂N(X¹)X² or N(X¹)X², wherein X¹ and X² are each, independently, H, aryl, C₁-C₆-alkyl, esters thereof; salts thereof, and any combination thereof; Z is selected from the group consisting of —H, —OH, —(CH₂)₀₋₆Y, —O—(CH₂)₀₋₆Y, wherein Y is selected from —H, —OH, —CN, halo, —CHCH₂, —CH₂CHCH₂, —NO₂, morpholinyl, hydroxyphenyl, phenyl, piperazinyl, cyclopropyl, cyclopentyl, cyclohexyl, pyridinyl, 5H-tetrazolyl, triazolyl, piperidinyl, alkylcarbonyl, alkylthiocarbonyl, alkoxycarbonyl, aminocarbonyl, —CO₂H, —SO₃H; —SO₂H; —SO₂NH₂, —SSO₃H, —PO₃H₂; —NO₂, —SH, —OSO₃H, —OC(O)(OH), —O—, —S—, halomethyl, dihalomethyl, trihalomethyl, —SO₂N(X¹)X² or N(X¹)X², wherein X¹ and X² are each, independently, H, aryl, C₁-C₆-alkyl, esters thereof; salts thereof, and any combination thereof, R⁷ and R^{7a} can also form together for a fused 5- or 6-membered ring composed of one of the following bridging bivalent radicals:
—O—CH₂—CH₂—
—CH₂—O—CH₂—
—CH₂—CH₂—O—
—CH₂—CH₂—CH₂—
—O—CH₂—O—
—CH₂—CH₂—CH₂—CH₂—

d and e are each, independently, 0 or 1; and f is 0, 1, 2, 3, 4, 5 or 6.

In a preferred embodiment of Formula 4, D is selected from the group consisting of O, N, or C₁₋₃-alkyl. In another preferred embodiment of Formula 4, Z is selected from the group consisting of —H, cyclohexyl, cyclopentyl, phenyl, —CO₂H or piperidinyl. In another embodiment of Formula 4, R⁶ is selected from the group consisting of —CHCH₂, or (CH₂)₁₋₃Y, wherein Y is selected from the group consisting of —H, —OH, Br or CO₂H. In yet another embodiment of Formula 4, R⁷ and R^{7a} are each, independently, selected from the group consisting of —H, —OCH₃, —NH₂ or —Br. In still another embodiment of Formula 4, R⁷ and R^{7a} form together for a fused 5-membered ring composed of the following bridging bivalent radical: —O—CH₂—O—. In another embodiment of Formula 4, f is 0-3. In still another embodiment of Formula 4, D is —O—, e is 1, and f is 1 or 2. In yet another embodiment of Formula 4, Z is selected from the group consisting of H, —COOH, cyclopropyl, cyclopentyl, n-butyl and t-butyl. In still another embodiment of Formula 4, R⁷ is —OCH₃, R^{7a} is H, and R³ and R⁴ are H. In another embodiment of Formula 4, R⁵ is H and R⁶ is —CHCH₂ or —CH₂CH₃.

Another preferred embodiment of Formula 1 is represented as Formula 5, 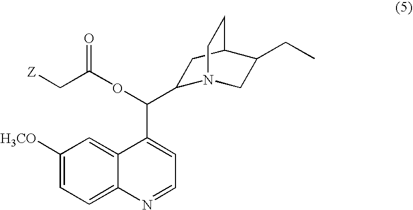
or a pharmaceutically acceptable salt thereof, wherein Z is selected from the group consisting of —H, —OH, —(CH₂)₀₋₆Y, —O—(CH₂)₀₋₆Y, wherein Y is selected from —H, C₁-C₆-alkyl, —OH, —CN, halo, —CHCH₂, —CH₂CHCH₂, —NO₂, morpholinyl, hydroxyphenyl, phenyl, piperazinyl, cyclopropyl, cyclopentyl, cyclohexyl, pyridinyl, 5H-tetrazolyl, triazolyl, piperidinyl, alkylcarbonyl, alkylthiocarbonyl, alkoxycarbonyl, aminocarbonyl, —CO₂H, —SO₃H; —SO₂H; —SO₂NH₂, —SSO₃H, —PO₃H₂; —NO₂, —SH, —OSO₃H, —OC(O)(OH), —O—, —S—, halomethyl, dihalomethyl, trihalomethyl, —SO₂N(X¹)X² or N(X¹)X², wherein X¹ and X² are each, independently, H, aryl, C₁-C₆-alkyl, esters thereof, salts thereof, and any combination thereof.

In a preferred embodiment of Formula 5, Z is selected from the group consisting of H, —COOH, cyclopropyl, cyclopentyl, CH₂CH₂CH₃, n-butyl and t-butyl.

A preferred embodiment of Formula 4 is 3-cyclopentyl-propionic acid quinolin-4-yl-(5-vinyl-1-aza-bicyclo[2.2.2]oct-2-yl)-methyl ester: 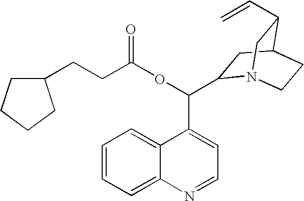
and pharmaceutically acceptable salts thereof.

Another preferred embodiment of Formula 4 is cyclopropanecarboxylic acid quinolin-4-yl-(5-vinyl-1-aza-bicyclo[2.2.2]oct-2-yl)-methyl ester: 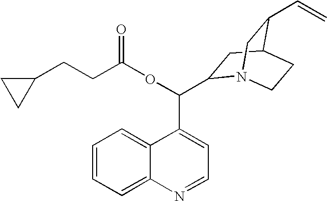
and pharmaceutically acceptable salts thereof.

Another preferred embodiment of Formula 4 is succinic acid mono-[quinolin-4-yl-(5-vinyl-1-aza-bicyclo[2.2.2]oct-2-yl)-methyl]ester: 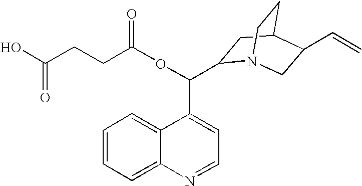
and pharmaceutically acceptable salts thereof.

Another preferred embodiment of Formula 4 is 3-cyclopentyl-propionic acid (6-methoxy-quinolin-4-yl)-(5-vinyl-1-aza-bicyclo[2.2.2]oct-2-yl)-methyl ester: 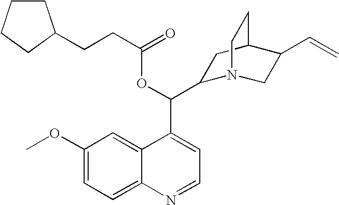
and pharmaceutically acceptable salts thereof (e.g., 2-[(3-cyclopentyl-propionyloxy)-(6-methoxy-quinolin-4-yl)-methyl]-5-vinyl-1-azonia-bicyclo[2.2.2]octane dihydrochloride).

Another preferred embodiment of Formula 4 is 3-cyclopentyl-propionic acid (5-ethyl-1-aza-bicyclo[2.2.2]oct-2-yl)-(6-methoxy-quinolin-4-yl)-methyl ester: 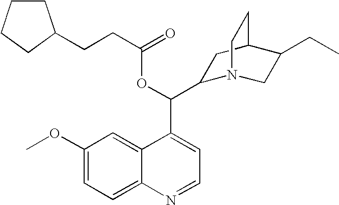
and pharmaceutically acceptable salts thereof (e.g., 2-[(3-cyclopentyl-propionyloxy)-(6-methoxy-quinolin-4-yl)-methyl]-5-ethyl-1-azonia-bicyclo[2.2.2]octane dihydrochloride).

Another preferred embodiment of Formula 4 is pentanoic acid (6-methoxy-quinolin-4-yl)-(5-vinyl-1-aza-bicyclo[2.2.2]oct-2-yl)-methyl ester: 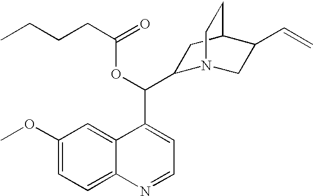
and pharmaceutically acceptable salts thereof (e.g., 2-[(6-methoxy-quinolin-4-yl)-pentanoyloxy-methyl]-5-vinyl-1-azonia-bicyclo[2.2.2]octane dihydrochloride).

Another preferred embodiment of Formula 4 is pentanoic acid (5-ethyl-1-aza-bicyclo[2.2.2]oct-2-yl)-(6-methoxy-quinolin-4-yl)-methyl ester: 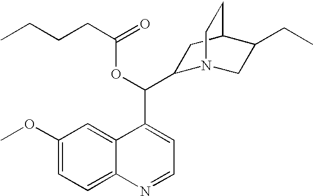
and pharmaceutically acceptable salts thereof (e.g., 5-ethyl-2-[(6-methoxy-quinolin-4-yl)-pentanoyloxy-methyl]-1-azonia-bicyclo[2.2.2]octane dihydrochloride).

Another preferred embodiment of Formula 4 is 3,3-dimethyl-butyric acid (6-methoxy-quinolin-4-yl)-(5-vinyl-1-aza-bicyclo[2.2.2]oct-2-yl)-methyl ester: 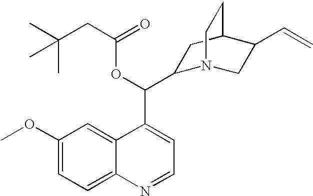
and pharmaceutically acceptable salts thereof (e.g., 2-[(3,3-dimethyl-butyryloxy)-(6-methoxy-quinolin-4-yl)-methyl]-5-vinyl-1-azonia-bicyclo[2.2.2]octane dihydrochloride).

Another preferred embodiment of Formula 4 is 3,3-dimethyl-butyric acid (5-ethyl-1-aza-bicyclo[2.2.2]oct-2-yl)-(6-methoxy-quinolin-4-yl)-methyl ester: 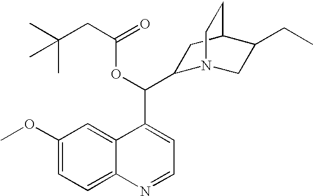
and pharmaceutically acceptable salts thereof (e.g., 2-[(3,3-dimethyl-butyryloxy)-(6-methoxy-quinolin-4-yl)-methyl]-5-ethyl-1-azonia-bicyclo[2.2.2]octane dihydrochloride).

It is to be understood that all of the compounds of Formula 1, 2, 3 and 4 described above will further include double bonds between adjacent atoms as required to satisfy the valence of each atom. That is, double bonds are added to provide the following number of total bonds to each of the following types of atoms: carbon: four bonds; nitrogen: three bonds; oxygen: two bonds; and sulfur: two bonds.

It will be noted that the structures of some of the compounds of this invention include asymmetric carbon atoms. It is to be understood accordingly that the isomers arising from such asymmetry (e.g., all enantiomers and diastereomers) are included within the scope of this invention, unless indicated otherwise. Such isomers can be obtained in substantially pure form by classical separation techniques and by stereochemically controlled synthesis. Furthermore, the structures and other compounds and moieties discussed in this application also include all tautomers thereof. Compounds described herein may be obtained though art recognized synthesis strategies.

In one embodiment of the invention, the compounds of the invention that modulate the activity of a gated ion channel are capable of chemically interacting with a gated ion channel, including αENaC, βENaC, γENaC, δENaC, ASIC1a, ASIC1b, ASIC2a, ASIC2b, ASIC3, ASIC4, BLINaC, hINaC, P2X₁, P2X₂, P2X₃, P2X₄, P2X₅, P2X₆, P2X₇, TRPV1, TRPV2, TRPV3, TRPV4, TRPV5, TRPV6. The language “chemical interaction” is intended to include, but is not limited to reversible interactions such as hydrophobic/hydrophilic, ionic (e.g. coulombic attraction/repulsion, ion-dipole, charge-transfer), covalent bonding, Van der Waals, and hydrogen bonding. In certain embodiments, the chemical interaction is a reversible Michael addition. In a specific embodiment, the Michael addition involves, at least in part, the formation of a covalent bond.

Compounds of the inventions can be synthesized according to standard organic synthesis procedures that are known in the art.

Below is a scheme for a general embodiment of Formula 4 using organic starting materials and synthesis procedures well-known in organic chemistry synthesis:

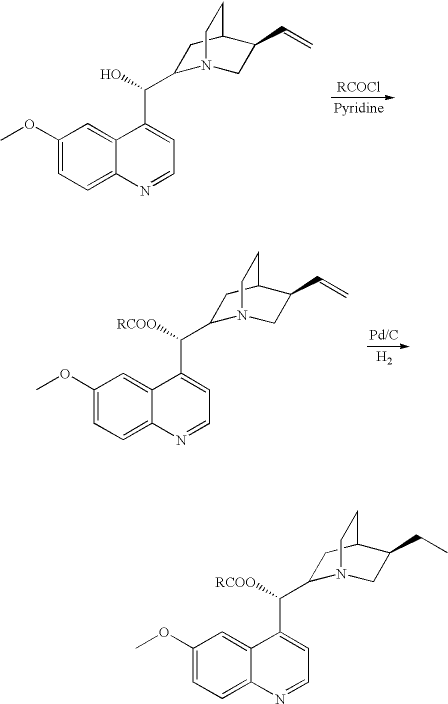

Below is a scheme for a specific embodiment of Formula 4 using organic starting materials and synthesis procedures well-known in organic chemistry synthesis: 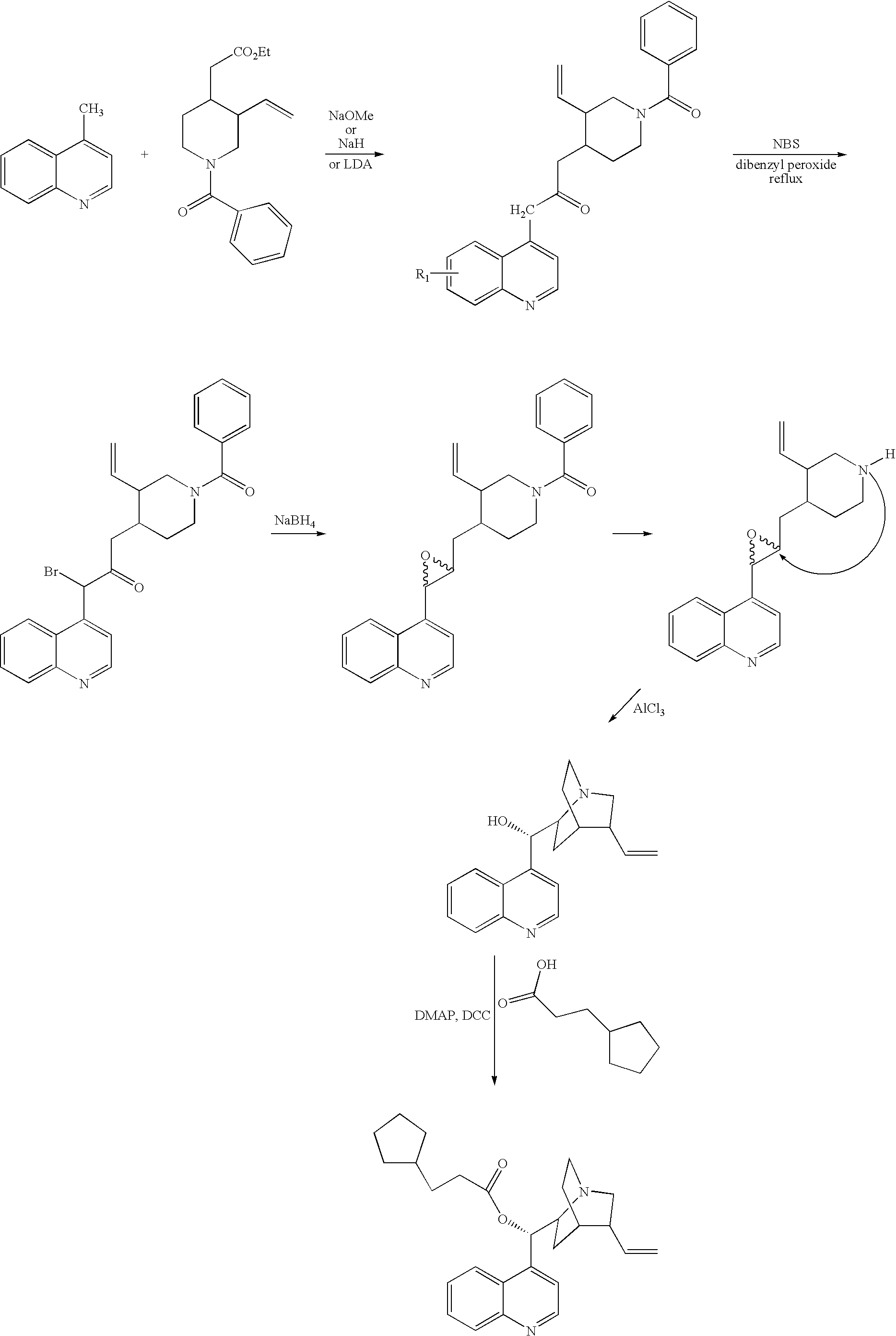
An analogous procedure can be found in German Patent No. 1933600.

The end products of the reactions described herein may be isolated by conventional techniques, e.g. by extraction, crystallisation, distillation, chromatography, etc.

Acid addition salts of the compounds of Formula 1 are most suitably formed from pharmaceutically acceptable acids, and include for example those formed with inorganic acids e.g. hydrochloric, sulphuric or phosphoric acids and organic acids e.g. succinic, maleic, acetic or fumaric acid. Other non-pharmaceutically acceptable salts e.g. oxalates may be used for example in the isolation of the compound of Formula 1, 2, 3 and 4 for laboratory use, or for subsequent conversion to a pharmaceutically acceptable acid addition salt. Also included within the scope of the invention are solvates and hydrates of the invention.

The conversion of a given compound salt to a desired compound salt is achieved by applying standard techniques, in which an aqueous solution of the given salt is treated with a solution of base e.g. sodium carbonate or potassium hydroxide, to liberate the free base which is then extracted into an appropriate solvent, such as ether. The free base is then separated from the aqueous portion, dried, and treated with the requisite acid to give the desired salt.

In vivo hydrolyzable esters or amides of certain compounds of Formula 1, 2, 3 and 4 can be formed by treating those compounds having a free hydroxy or amino functionality with the acid chloride of the desired ester in the presence of a base in an inert solvent such as methylene chloride or chloroform. Suitable bases include triethylamine or pyridine. Conversely, compounds of Formula 1, 2, 3 and 4 having a free carboxy group may be esterified using standard conditions which may include activation followed by treatment with the desired alcohol in the presence of a suitable base.

Examples of pharmaceutically acceptable addition salts include, without limitation, the non-toxic inorganic and organic acid addition salts such as the hydrochloride derived from hydrochloric acid, the hydrobromide derived from hydrobromic acid, the nitrate derived from nitric acid, the perchlorate derived from perchloric acid, the phosphate derived from phosphoric acid, the sulphate derived from sulphuric acid, the formate derived from formic acid, the acetate derived from acetic acid, the aconate derived from aconitic acid, the ascorbate derived from ascorbic acid, the benzenesulphonate derived from benzensulphonic acid, the benzoate derived from benzoic acid, the cinnamate derived from cinnamic acid, the citrate derived from citric acid, the embonate derived from embonic acid, the enantate derived from enanthic acid, the fumarate derived from fumaric acid, the glutamate derived from glutamic acid, the glycolate derived from glycolic acid, the lactate derived from lactic acid, the maleate derived from maleic acid, the malonate derived from malonic acid, the mandelate derived from mandelic acid, the methanesulphonate derived from methane sulphonic acid, the naphthalene-2-sulphonate derived from naphtalene-2-sulphonic acid, the phthalate derived from phthalic acid, the salicylate derived from salicylic acid, the sorbate derived from sorbic acid, the stearate derived from stearic acid, the succinate derived from succinic acid, the tartrate derived from tartaric acid, the toluene-p-sulphonate derived from p-toluene sulphonic acid, and the like. Such salts may be formed by procedures well known and described in the art.

Other acids such as oxalic acid, which may not be considered pharmaceutically acceptable, may be useful in the preparation of salts useful as intermediates in obtaining a chemical compound of the invention and its pharmaceutically acceptable acid addition salt.

Metal salts of a chemical compound of the invention includes alkali metal salts, such as the sodium salt of a chemical compound of the invention containing a carboxy group.

In the context of this invention the “onium salts” of N-containing compounds are also contemplated as pharmaceutically acceptable salts. Preferred “onium salts” include the alkyl-onium salts, the cycloalkyl-onium salts, and the cycloalkylalkyl-onium salts.

The chemical compound of the invention may be provided in dissoluble or indissoluble forms together with a pharmaceutically acceptable solvents such as water, ethanol, and the like. Dissoluble forms may also include hydrated forms such as the monohydrate, the dihydrate, the hemihydrate, the trihydrate, the tetrahydrate, and the like. In general, the dissoluble forms are considered equivalent to indissoluble forms for the purposes of this invention.

A. Stereoisomers

The chemical compounds of the present invention may exist in (+) and (−) forms as well as in racemic forms. The racemates of these isomers and the individual isomers themselves are within the scope of the present invention.

Racemic forms can be resolved into the optical antipodes by known methods and techniques. One way of separating the diastereomeric salts is by use of an optically active acid, and liberating the optically active amine compound by treatment with a base. Another method for resolving racemates into the optical antipodes is based upon chromatography on an optical active matrix. Racemic compounds of the present invention can thus be resolved into their optical antipodes, e.g., by fractional crystallisation of d- or I-(tartrates, mandelates, or camphorsulphonate) salts for example.

The chemical compounds of the present invention may also be resolved by the formation of diastereomeric amides by reaction of the chemical compounds of the present invention with an optically active activated carboxylic acid such as that derived from (+) or (−) phenylalanine, (+) or (−) phenylglycine, (+) or (−) camphanic acid or by the formation of diastereomeric carbamates by reaction of the chemical compound of the present invention with an optically active chloroformate or the like.

Additional methods for the resolving the optical isomers are known in the art. Such methods include those described by Jaques J, Collet A, and Wilen S in “Enantiomers, Racemates, and Resolutions”, John Wiley and Sons, New York (1981).

Optical active compounds can also be prepared from optical active starting materials.

Moreover, some of the chemical compounds of the invention being oximes, may thus exist in two forms, syn- and anti-form (Z- and E-form), depending on the arrangement of the substituents around the —C═N— double bond. A chemical compound of the present invention may thus be the syn- or the anti-form (Z- and E-form), or it may be a mixture hereof.

The term “alkyl” includes saturated aliphatic groups, including straight-chain alkyl groups (e.g., methyl, ethyl, propyl, butyl, pentyl, hexyl, heptyl, octyl, nonyl, decyl, etc.), branched-chain alkyl groups (isopropyl, tert-butyl, isobutyl, etc.), cycloalkyl (alicyclic) groups (cyclopropyl, cyclopentyl, cyclohexyl, cycloheptyl, cyclooctyl), alkyl substituted cycloalkyl groups, and cycloalkyl substituted alkyl groups.

The term alkyl further includes alkyl groups, which can further include oxygen, nitrogen, sulfur or phosphorous atoms replacing one or more carbons of the hydrocarbon backbone. In an embodiment, a straight chain or branched chain alkyl has 10 or fewer carbon atoms in its backbone (e.g., C₁-C₁₀ for straight chain, C₃-C₁₀ for branched chain), and more preferably 6 or fewer. Likewise, preferred cycloalkyls have from 4-7 carbon atoms in their ring structure, and more preferably have 5 or 6 carbons in the ring structure.

In the context of this invention halogen represents a fluorine, a chlorine, a bromine or an iodine atom. Thus, a trihalogenmethyl group represents e.g. a trifluoromethyl group and a trichloromethyl group.

The term “substituted” is intended to describe moieties having substituents replacing a hydrogen on one or more atoms, e.g. C or N, of a molecule. Such substituents can include, for example, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, morpholino, hydroxyphenyl, phenyl, piperizine, cyclopentane, cyclohexane, pyridine, 5H-tetrazole, triazole, piperidine, or an aromatic or heteroaromatic moiety.

The term “aryl” includes groups, including 5- and 6-membered single-ring aromatic groups that may include from zero to four heteroatoms, for example, phenyl, pyrrole, furan, thiophene, thiazole, isothiaozole, imidazole, triazole, tetrazole, pyrazole, oxazole, isoxazole, pyridine, pyrazine, pyridazine, and pyrimidine, and the like. Furthermore, the term “aryl” includes multicyclic aryl groups, e.g., tricyclic, bicyclic, e.g., naphthalene, benzoxazole, benzodioxazole, benzothiazole, benzoimidazole, benzothiophene, methylenedioxyphenyl, quinoline, isoquinoline, napthridine, indole, benzofuran, purine, benzofuran, deazapurine, or indolizine. Those aryl groups having heteroatoms in the ring structure may also be referred to as “aryl heterocycles”, “heterocycles,” “heteroaryls” or “heteroaromatics”. The aromatic ring can be substituted at one or more ring positions with such substituents as described above, as for example, halogen, hydroxyl, alkoxy, alkylcarbonyloxy, arylcarbonyloxy, alkoxycarbonyloxy, aryloxycarbonyloxy, carboxylate, alkylcarbonyl, alkylaminoacarbonyl, aralkylaminocarbonyl, alkenylaminocarbonyl, alkylcarbonyl, arylcarbonyl, aralkylcarbonyl, alkenylcarbonyl, alkoxycarbonyl, aminocarbonyl, alkylthiocarbonyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety. Aryl groups can also be fused or bridged with alicyclic or heterocyclic rings which are not aromatic so as to form a polycycle (e.g., tetralin).

Additionally, the phrase “any combination thereof” implies that any number of the listed functional groups and molecules may be combined to create a larger molecular architecture. For example, the terms “phenyl,” “carbonyl” (or “═O”), “—O—,” “—OH,” and C₁₋₆ (i.e., —CH₃ and —CH₂CH₂CH₂—) can be combined to form a 3-methoxy-4-propoxybenzoic acid substituent. It is to be understood that when combining functional groups and molecules to create a larger molecular architecture, hydrogens can be removed or added, as required to satisfy the valence of each atom.

II. Assays

The present invention relates to a method of modulating gated ion channel activity. As used herein, the various forms of the term “modulate” include stimulation (e.g., increasing or upregulating a particular response or activity) and inhibition (e.g., decreasing or downregulating a particular response or activity). In one aspect, the methods of the present invention comprise contacting a cell with an effective amount of a gated ion channel modulator compound, e.g. a compound of the invention, thereby modulating the activity of a gated ion channel. In certain embodiments, the effective amount of the compound of the invention inhibits the activity of the gated ion channel

The gated ion channels of the present invention are comprised of at least one subunit belonging to the DEG/ENaC, TRPV (also referred to as vanilloid) and/or P2X gene superfamilies. In one aspect the gated ion channel is comprised of at least one subunit selected from the group consisting of αENaC, βENaC, γENaC, δENaC, ASIC1a, ASIC1b, ASIC2a, ASIC2b, ASIC3, ASIC4, BLINaC, hINaC,, P2X₁, P2X₂, P2X₃, P2X₄, P2X₅, P2X₆, P2X₇, TRPV1, TRPV2, TRPV3, TRPV4, TRPV5, and TRPV6. In one aspect, the DEG/ENaC gated ion channel is comprised of at least one subunit selected from the group consisting of αENaC, βENaC, γENaC, δENaC, BLINaC, hINaC, ASIC1a, ASIC1b, ASIC2a, ASIC2b, ASIC3, and ASIC4. In certain embodiments, the DEG/ENaC gated ion channel is comprised of at least one subunit selected from the group consisting of ASIC1a, ASIC1b, ASIC2a, ASIC2b, ASIC3, and ASIC4. In certain embodiments, the gated ion channel is comprised of ASIC1a, ASIC1b, or ASIC3. In another aspect of the invention, P2X gated ion channel is comprised of at least one subunit selected from the group consisting of P2X₁, P2X₂, P2X₃, P2X₄, P2X₅, P2X₆, and P2X₇. In yet another aspect of the invention, the TRPV gated ion channel is comprised of at least one subunit selected from the group TRPV1, TRPV2, TRPV3, TRPV4, TRPV5, and TRPV6. In another apect, the gated ion channel is a heteromultimeric gated ion channel, including, but not limited to, αENaC, βENaC and γENaC; αENaC, βENaC and δENaC; ASIC1a and ASIC2a; ASIC1a and ASIC2b; ASIC1a and ASIC3; ASIC1b and ASIC3; ASIC2a and ASIC2b; ASIC2a and ASIC3; ASIC2b and ASIC3; ASIC1a, ASIC2a and ASIC3; ASIC3 and P2X, e.g P2X1, P2X2, P2X3, P2X4, P2X5, P2X6 and P2X7, preferably ASIC3 and P2X2; ASIC3 and P2X3; and ASIC3, P2X2 and P2X3; ASIC4 and at least one of ASIC1a, ASIC1b, ASIC2a, ASIC2b, and ASIC3; BLINaC (or hINaC) and at least one of ASIC1a, ASIC1b, ASIC2a, ASIC2b, ASIC3, and ASIC4; 6ENaC and ASIC, e.g. ASIC1a, ASIC1b, ASIC2a, ASIC2b, ASIC3 and ASIC4; P2X1 and P2X2, P2X1 and P2X5, P2X2 and P2X3, P2X2 and P2X6, P2X4 and P2X6, TRPV1 and TRPV2, TRPV5 and TRPV6, TRPV1 and TRPV4.

Assays for determining the ability of a compound within the scope of the invention to modulate the activity of gated ion channels are well known in the art and described in Examples 1-4. Other assays for determining the ability of a compound to modulate the activity of a gated ion channel are also readily available to the skilled artisan.

The gated ion channel modulating compounds of the invention may be identified using the following screening method, which method comprises the subsequent steps of

(i) subjecting a gated ion channel containing cell to the action of a selective activator, e.g., protons by adjustment of the pH to an acidic level, ATP by diluting sufficient amounts of ATP in the perfusion buffer or temperature by heating the perfusion buffer to temperatures above 37° C.;

(ii) subjecting a gated ion channel containing cell to the action of the chemical compound; and

(iii) monitoring the change in membrane potential or ionic current induced by the activator, e.g., protons, on the gated ion channel containing cell. Alternatively, fluorescent imaging can be utilized to monitor the effect induced by the activator, e.g., protons, on the gated ion channel containing cell.

The gated ion channel containing cells may be subjected to the action of protons by adjustment of the pH to an acidic level using any convenient acid or buffer, including organic acids such as formic acid, acetic acid, citric acid, ascorbic acid, 2-Morpholinoethanesulfonic acid (MES) and lactic acid, and inorganic acids such as hydrochloric acid, hydrobromic acid and nitric acid, perchloric acid and phosphoric acid.

In the methods of the invention, the current flux induced by the activator, e.g., protons, across the membrane of the gated ion channel containing cell may be monitored by electrophysiological methods, for example patch clamp or two-electrode voltage clamp techniques.

Alternatively, the change in membrane potential induced by gated ion channel activators, e.g., protons of the gated ion channel containing cells may be monitored using fluorescence methods. When using fluorescence methods, the gated ion channel containing cells are incubated with a membrane potential indicating agent that allows for a determination of changes in the membrane potential of the cells, caused by the added activators, e.g., protons. Such membrane potential indicating agents include fluorescent indicators, preferably DiBAC₄(3), DiOC5(3), DiOC2(3), DiSBAC2(3) and the FMP dye (Molecular Devices).

In another alternative embodiment, the change in gated ion channel activity induced by activators, e.g., protons, on the gated ion channel can be measured by assessing changes in the intracellular concentration of certain ions, e.g., calcium, sodium, potassium, magnesium, protons, and chloride in cells by fluorescence. Fluorescence assays can be performed in multi-well plates using plate readers, e.g., FLIPR assay (Fluorescence Image Plate Reader; available from Molecular Devices), e.g. using fluorescent calcium indicators, e.g. as described in, for example, Sullivan E., et al. (1999) Methods Mol Biol. 114:125-33, Jerman, J. C., et al. (2000) Br J Pharmacol 130(4):916-22, and U.S. Pat. No. 6,608,671, the contents of each of which are incorporated herein by reference. When using such fluorescence methods, the gated ion channel containing cells are incubated with a selective ion indicating agent that allows for a determination of changes in the intracellular concentration of the ion, caused by the added activators, e.g., protons. Such ion indicating agents include fluorescent calcium indicators, preferably Fura-2, Fluo-3, Fluo-4, Fluo4FF, Fluo-5F, Fluo-5N, Calcium Green, Fura-Red, Indo-1, Indo-5F, and rhod-2, fluorescent sodium indicators, preferably SBFI, Sodium Green, CoroNa Green, fluorescent potassium indicators, preferably PBFI, CD222, fluorescent magnesium indicators, preferably Mag-Fluo-4, Mag-Fura-2, Mag-Fura-5, Mag-Fura-Red, Mag-indo-1, Mag-rho-2, Magnesium Green, fluorescent chloride indicators, preferably SPQ, Bis-DMXPQ, LZQ, MEQ, and MQAE, fluorescent pH indicators, preferably BCECF and BCPCF.

The gated ion channel antagonising compounds of the invention show activity in concentrations below 2M, 1.5M, 1 M, 500 mM, 250 mM, 100 mM, 750 μM, 500 μM, 250 μM, 100 μM, 75 μM, 50 μM, 25 μM, 10 μM, 5 μM, 2.5 μM, or below 1 μm. In its most preferred embodiment the ASIC antagonising compounds show activity in low micromolar and the nanomolar range.

As used herein, the term “contacting” (i.e., contacting a cell e.g. a neuronal cell, with a compound) is intended to include incubating the compound and the cell together in vitro (e.g., adding the compound to cells in culture) or administering the compound to a subject such that the compound and cells of the subject are contacted in vivo. The term “contacting” is not intended to include exposure of cells to a modulator or compound that may occur naturally in a subject (i.e., exposure that may occur as a result of a natural physiological process).

A. In Vitro Assays

Gated ion channel polypeptides for use in the assays described herein can be readily produced by standard biological techniques or by chemical synthesis. For example, a host cell transfected with an expression vector containing a nucleotide sequence encoding the desired gated ion channel can be cultured under appropriate conditions to allow expression of the peptide to occur. Alternatively, the gated ion channel can be obtained by culturing a primary cell line or an established cell line that can produce the gated ion channel.

The methods of the invention can be practiced in vitro, for example, in a cell-based culture screening assay to screen compounds which potentialy bind, activate or modulate gated ion channel function. In such a method, the modulating compound can function by interacting with and eliminating any specific function of gated ion channel in the sample or culture. The modulating compounds can also be used to control gated ion channel activity in neuronal cell culture.

Cells for use in in vitro assays, in which gated ion channels are naturally present, include various cell lines, such as cortical neuronal cell lines, in particular mouse or rat cortical neuronal cells, and human embryonic kidney (HEK) cells, in particular HEK293 cells. Primary cell cultures can also be used in the methods of the invention. For example, sensory neuronal cells can also be isolated and cultured in vitro from different animal species. The most widely used protocols use sensory neurons isolated from neonatal (Eckert, et al. (1997) J Neurosci Methods 77:183-190) and embryonic (Vasko, et al. (1994) J Neurosci 14:4987-4997) rat. Trigeminal and dorsal root ganglion sensory neurons in culture exhibit certain characteristics of sensory neurons in vivo.

Alternatively, the gated ion channel, e.g., a gated channel, e.g., a proton gated ion channel, may be exogenous to the cell in question, and may in particular be introduced by recombinant DNA technology, such as transfection, microinjection or infection. Such cells include Chinese hamster ovary (CHO) cells, African green monkey kidney cell line (CV-1 or CV-1-derived COS cells, e.g. COS-1 and COS-7) Xenopus laevis oocytes, or any other cell lines capable of expressing gated ion channels.

The nucleotide and amino acid sequences of the gated ion channels of the invention are known in the art. For example, the sequences of the human gated channels can be found in Genbank GI Accession Nos: GI:40556387 (ENaCalpha Homo sapiens); GI:4506815 (ENaCalpha Homo sapiens); GI:4506816 (ENaCbeta Homo sapiens); GI:4506817 (ENaCbeta Homo sapiens); GI:34101281 (ENaCdelta Homo sapiens); GI:34101282 (ENaCdelta Homo sapiens); GI:42476332 (ENaCgamma Homo sapiens); GI:42476333 (ENaCgamma Homo sapiens); GI:31442760 (HINAC Homo sapiens); GI:31442761 (HINAC Homo sapiens); GI: 21536350 (ASIC1a Homo sapiens); GI:21536351 (ASIC1a Homo sapiens); GI:21536348(ASIC1b Homo sapiens); GI:21536349 (ASIC1b Homo sapiens); GI:34452694 (ASIC2; transcript variant 1 Homo sapiens); GI:34452695 (ASIC2; isoform 1 Homo sapiens); GI:34452696(ASIC2; transcript variant 2 Homo sapiens); GI:9998944 (ASIC2; isoform 2 Homo sapiens); GI:4757709 (ASIC3; transcript variant 1 Homo sapiens); GI:4757710(ASIC3; isoform 1 Homo sapiens); GI:9998945(ASIC3; transcript variant 2 Homo sapiens); GI:9998946 (ASIC3; isoform 2 Homo sapiens); GI:9998947 (ASIC3; transcript variant 3 Homo sapiens); GI: 9998948 (ASIC3; isoform 3 Homo sapiens); GI:33519441 (ASIC4; transcript variant 1 Homo sapiens); GI:33519442 (ASIC4; isoform 1 Homo sapiens); GI:33519443 (ASIC4; transcript variant 2 Homo sapiens); GI:33519444 (ASIC4; isoform 2 Homo sapiens); GI:27894283 (P2X1 Homo sapiens); GI:4505545 (P2X1 Homo sapiens); GI:28416917 (P2X2; transcript variant 1 Homo sapiens); GI:25092719 (P2X2; isoform A Homo sapiens); GI:28416922(P2X2; transcript variant 2 Homo sapiens); GI:28416923 (P2X2; isoform B Homo sapiens); GI:28416916(P2X2; transcript variant 3 Homo sapiens); GI:7706629 (P2X2; isoform C Homo sapiens); GI:28416918(P2X2; transcript variant 4 Homo sapiens); GI:25092733 (P2X2; isoform D Homo sapiens); GI:28416920 (P2X2; transcript variant 5 Homo sapiens); GI:28416921(P2X2; isoform H Homo sapiens); GI:28416919 (P2X2; transcript variant 6 Homo sapiens); GI:27881423 (P2X2; isoform 1 Homo sapiens); GI:28416924 (P2X3 Homo sapiens); GI:28416925 (P2X3 Homo sapiens); GI:28416926 (P2X4; transcript variant 1 Homo sapiens); GI:28416927 (P2X4; isoform A Homo sapiens); GI: 28416928 (P2X4; transcript variant 2 Homo sapiens); GI:28416929 (P2X4; isoform B Homo sapiens); GI:28416930 (P2X4; transcript variant 3 Homo sapiens); GI:2841693 1(P2X4; isoform C Homo sapiens); GI:28416932 (P2X5; transcript variant 1 Homo sapiens); GI:28416933(P2X5; isoform A Homo sapiens); GI:28416934 (P2X5; transcript variant 2 Homo sapiens); GI:28416935 (P2X5; isoform B Homo sapiens); GI:28416936 (P2X5; transcript variant 3 Homo sapiens); GI:28416937 (P2X5; isoform C Homo sapiens); GI:38327545 (P2X6 Homo sapiens); GI:4885535 (P2X6 Homo sapiens); GI:34335273 (P2X7; transcript variant 1 Homo sapiens); GI:29294631 (P2X7; isoform A Homo sapiens); GI:34335274 (P2X7; transcript variant 2 Homo sapiens); GI:29294633 (P2X7; isoform B Homo sapiens); GI:18375666 (TRPV1; transcript variant 1 Homo sapiens); GI:18375667(TRPV1; vanilloid receptor subtype 1 Homo sapiens); GI:18375664 (TRPV 1; transcript variant 2 Homo sapiens); GI:18375665 (TRPV 1; vanilloid receptor subtype 1 Homo sapiens); GI:18375670 (TRPV1; transcript variant 3 Homo sapiens); GI:18375671(TRPV1; vanilloid receptor subtype 1 Homo sapiens); GI:18375668 (TRPV1; transcript variant 4 Homo sapiens); GI:18375669 (TRPV1; vanilloid receptor subtype 1 Homo sapiens); GI:7706764 (VRL-1; transcript variant 1 Homo sapiens); GI:7706765 (VRL-1; vanilloid receptor-like protein 1 Homo sapiens); GI:22547178 (TRPV2; transcript variant 2 Homo sapiens); GI:20127551 (TRPV2; vanilloid receptor-like protein 1 Homo sapiens); GI:22547183 (TRPV4; transcript variant 1 Homo sapiens); GI:22547184 (TRPV4; isoform A Homo sapiens); GI:22547179 (TRPV4; transcript variant 2 Homo sapiens); GI:22547180 (TRPV4; isoform B Homo sapiens); GI:21361832 (TRPV5 Homo sapiens); GI:17505200 (TRPV5 Homo sapiens); GI:21314681 (TRPV6 Homo sapiens); GI:21314682 (TRPV6 Homo sapiens); GI: 34452696 (ACCN1; transcript variant 2; Homo sapiens); GI: 34452694 (ACCN1; transcript variant 1; Homo sapiens); GI: 9998944 (ACCN1; isoform 2; Homo sapiens); GI: 34452695 (ACCN1; isoform 1; Homo sapiens). The contents of each of these records is incorporated herein by reference. Additionally, sequences for channels of other species are readily available and obtainable by those skilled in the art.

A nucleic acid molecule encoding a gated ion channel for use in the methods of the present invention can be amplified using cDNA, mRNA, or genomic DNA as a template and appropriate oligonucleotide primers according to standard PCR amplification techniques. The nucleic acid so amplified can be cloned into an appropriate vector and characterized by DNA sequence analysis. Using all or a portion of such nucleic acid sequences, nucleic acid molecules of the invention can be isolated using standard hybridization and cloning techniques (e.g., as described in Sambrook et al., ed., Molecular Cloning: A Laboratory Manual, 2nd edt, Cold Spring Harbor Laboratory Press, Cold Spring Harbor, N.Y., 1989).

Expression vectors, containing a nucleic acid encoding a gated ion channel, e.g., a gated ion channel subunit protein, e.g., αENaC, βENaC, γENaC, δENaC, ASIC1a, ASIC1b, ASIC2a, ASIC2b, ASIC3, ASIC4, BLINaC, hINaC, P2X₁, P2X₂, P2X₃, P2X₄, P2X₅, P2X₆, P2X₇, TRPV1, TRPV2, TRPV3, TRPV4, TRPV5, and TRPV6 protein (or a portion thereof) are introduced into cells using standard techniques and operably linked to regulatory sequence. Such regulatory sequences are described, for example, in Goeddel, Methods in Enzymology: Gene Expression Technology vol. 185, Academic Press, San Diego, Calif. (1991). Regulatory sequences include those which direct constitutive expression of a nucleotide sequence in many types of host cell and those which direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, and the like. The expression vectors of the invention can be introduced into host cells to thereby produce proteins or peptides, including fusion proteins or peptides, encoded by nucleic acids as described herein.

Examples of vectors for expression in yeast S. cerevisiae include pYepSec1 (Baldari et al., 1987, EMBO J. 6:229-234), pMFa (Kurjan and Herskowitz, 1982, Cell 30:933-943), pJRY88 (Schultz et al., 1987, Gene 54:113-123), pYES2 (Invitrogen Corporation, San Diego, Calif.), and pPicZ (Invitrogen Corp, San Diego, Calif.).

Baculovirus vectors available for expression of proteins in cultured insect cells (e.g., Sf 9 cells) include the pAc series (Smith et al., 1983, Mol. Cell Biol. 3:2156-2165) and the pVL series (Lucklow and Summers, 1989, Virology 170:31-39).

Examples of mammalian expression vectors include pCDM8 (Seed, 1987, Nature 329:840), pMT2PC (Kaufman et al., 1987, EMBO J. 6:187-195), pCDNA3. When used in mammalian cells, the expression vector's control functions are often provided by viral regulatory elements. For example, commonly used promoters are derived from polyoma, Adenovirus 2, cytomegalovirus and Simian Virus 40. For other suitable expression systems for eukaryotic cells see chapters 16 and 17 of Sambrook et al., supra.

B. In Vivo Assays

The activity of the compounds of the invention as described herein to modulate one or more gated ion channel activies (e.g., a gated ion channel modulator, e.g., a compound of the invention) can be assayed in an animal model to determine the efficacy, toxicity, or side effects of treatment with such an agent. Alternatively, an agent identified as described herein can be used in an animal model to determine the mechanism of action of such an agent.

Animal models for determining the ability of a compound of the invention to modulate a gated ion channel biological activity are well known and readily available to the skilled artisan. Examples of animal models for pain and inflammation include, but are not limited to the models listed in Table 5. Animal models for investigating neurological disorders include, but are not limited to, those described in Morris et al., (Learn. Motiv. 1981; 12: 239-60) and Abeliovitch et al., Cell 1993; 75: 1263-71). An example of an animal model for investigating mental and behavioral disorders is the Geller-Seifter paradigm, as described in Psychopharmacology (Berl). 1979 Apr. 11;62(2):117-21.

Genitourinary models include methods for reducing the bladder capacity of test animals by infusing either protamine sulfate and potassium chloride (See, Chuang, Y. C. et al., Urology 61(3): 664-670 (2003)) or dilute acetic acid (See, Sasaki, K. et al., J. Urol. 168(3): 1259-1264 (2002)) into the bladder. For urinary tract disorders involving the bladder using intravesically administered protamine sulfate as described in Chuang et al. (2003) Urology 61: 664-70. These methods also include the use of a well accepted model of for urinary tract-disorders involving the bladder using intravesically administered acetic acid as described in Sasaki et al. (2002) J. Urol. 168: 1259-64. Efficacy for treating spinal cord injured patients can be tested using methods as described in Yoshiyama et al. (1999) Urology 54: 929-33.

For neurological models for studying spinal cord injury, see, Yoshiyama, M. et al., Urology 54(5): 929-933 (1999).

Gastrointestinal models can be found in: Gawad, K. A., et al., Ambulatory long-term pH monitoring in pigs, Surg Endosc, (2003); Johnson, S. E. et al., Esophageal Acid Clearance Test in Healthy Dogs, Can. J. Vet. Res. 53(2): 244-7 (1989); and Cicente, Y. et al., Esophageal Acid Clearance: More Volume-dependent Than Motility Dependent in Healthy Piglets, J. Pediatr. Gastroenterol. Nutr. 35(2): 173-9 (2002). Models for a variety of assays can be used to assess visceromotor and pain responses to rectal distension. See, for example, Gunter et al., Physiol. Behav., 69(3): 379-82 (2000), Depoortere et al., J. Pharmacol. and Exp. Ther., 294(3): 983-990 (2000), Morteau et al., Fund. Clin. Pharmacol., 8(6): 553-62 (1994), Gibson et al., Gastroenterology (Suppl. 1), 120(5): A19-A20 (2001) and Gschossmann et al., Eur. J. Gastro. Hepat., 14(10): 1067-72 (2002) the entire contents of which are each incorporated herein by reference.

Gastrointestinal motility can be assessed based on either the in vivo recording of mechanical or electrical events associated intestinal muscle contractions in whole animals or the activity of isolated gastrointestinal intestinal muscle preparations recorded in vitro in organ baths (see, for example, Yaun et al., Br. J. Pharmacol., 112(4):1095-1100 (1994), Jin et al., J. Pharm. Exp. Ther., 288(1): 93-97 (1999) and Venkova et al., J. Pharm. Exp. Ther., 300(3): 1046-1052 (2002)). Tatersall et al. and Bountra et al., European Journal of Pharmacology, 250: (1993) R5 and 249:(1993) R3-R4 and Milano et al., J. Pharmacol. Exp. Ther., 274(2): 951-961 (1995).

Alternatively, the compounds can also be assayed in non-human transgenic animals containing exogenous sequences encoding one or more gated ion channels. As used herein, a “transgenic animal” is a non-human animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, etc. Methods for generating transgenic animals via embryo manipulation and microinjection, particularly animals such as mice, have become conventional in the art and are described, for example, in U.S. Pat. Nos. 4,736,866 and 4,870,009, U.S. Pat. No. 4,873,191 and in Hogan, Manipulating the Mouse Embryo, Cold Spring Harbor Laboratory Press, Cold Spring Harbor, N.Y., 1986. Similar methods are used for production of other transgenic animals.

A homologous recombinant animal can also be used to assay the compounds of the invention. Such animals can be generated according to well known techniques (see, e.g., Thomas and Capecchi, 1987, Cell 51:503; Li et al., 1992, Cell 69:915; Bradley, Teratocarcinomas and Embryonic Stem Cells: A Practical Approach, Robertson, Ed., IRL, Oxford, 1987, pp. 113-152;Bradley (1991) Current Opinion in Bio/Technology 2:823-829 and in PCT Publication NOS. WO 90/11354, WO 91/01140, WO 92/0968, and WO 93/04169).

Other useful transgenic non-human animals can be produced which contain selected systems which allow for regulated expression of the transgene (see, e.g., Lakso et al. (1992) Proc. Natl. Acad. Sci. USA 89:6232-6236). Another example of a recombinase system is the FLP recombinase system of Saccharomyces cerevisiae (O'Gorman et al., 1991, Science 251:1351-1355).

III. Methods of Treatment

The compounds of the present invention modulate gated ion channel-associated diseases, disorders and conditions and are therefore useful as treatments for neurological diseases, disorders and conditions as described above. The compounds of the present invention are also useful for the treatment of pain. As used herein, a “gated ion channel modulator” refers to a compound that modulates, inhibits, promotes or otherwise alters the responsiveness of gated ion channels to, for example, variations of voltage, temperature, pH, intra- and extracellular ligand concentrations, intra- and extracellular ion concentrations, and/or mechanical stimulation. For example, an “ASIC1a modulator” refers to a compound that modulates, e.g., inhibits, promotes, or otherwise alters, an activity of a gated ion channel, e.g., an ASIC1a gated ion channel.

“Gated ion channel-mediated activity” is a biological activity that is normally modulated (e.g., promoted), either directly or indirectly, in the presence of a gated ion channel (e.g., an ASIC). A biological activity that is mediated by a particular gated ion channel, e.g. ASIC1a, is referred to herein by reference to that gated ion channel, e.g. ASIC1a-mediated activity. To determine the ability of a compound to inhibit a gated ion channel-mediated activity, conventional in vitro and in vivo assays can be used.

The present invention provides for both therapeutic methods of treating a subject at risk of (or susceptible to) a disease, disorder or condition or having a disease, disorder or condition associated with the activity of gated ion channels, e.g. inflammatory pain, acute pain, chronic malignant pain, chronic nonmalignant pain, neuropathic pain and migraine, and neurological, neurodegenerative or neuropsychiatric disorders. Accordingly, the present invention provides methods to treat a subject, e.g., a mammalian subject, e.g., a human, that would benefit from the modulation of an activity of a gated ion channel.

In one embodiment, the invention provides a method of treating a disease, disorder or condition mediated by a gated ion channel activity in a subject. The method comprises the step of administering to the subject a therapeutically effective amount of a gated ion channel modulator. The disease, disorder or condition to be treated can be any disease, disorder or condition which is modulated, at least in part, directly or indirectly, by interaction of a compound of the invention with gated ion channels, which would in turn diminish or alleviate at least one symptom associated with or caused by the gated ion channel-mediated activity being treated. For example, treatment can be diminishment of one or several symptoms of a disorder or complete eradication of a disorder.

In another embodiment, the invention provides a method for the treatment, prevention or alleviation of at least one symptom of a disease, disorder or condition in a subject, e.g., a mammalian subject, e.g., a human, wherein the disease, disorder or condition is associated with modulation of extracellular pH and comprises administering a therapeutically effective amount of a compound of the invention to the subject. In another embodiment, the invention provides a method for the treatment, prevention or alleviation of at least one symptom of a disease, disorder or condition in a subject, e.g., a mammalian subject, e.g., a human, wherein the disease, disorder or condition is associated with modulation of extracellular calcium and comprises administering a therapeutically effective amount of a compound of the invention to the subject.

In yet another embodiment, the invention provides a method for the treatment, prevention or alleviation of pain in a subject, e.g., a mammalian subject, e.g., a human, comprising administering a therapeutically effective amount of a compound of the invention to the subject.

In another embodiment, a method for the treatment, prevention or alleviation of at least one symptom of a neurological disease, disorder or condition in a subject, e.g., a mammalian subject, e.g., a human, comprising administering a therapeutically effective amount of a compound of the invention to the subject, is provided.

The terms “treated,” “treating” or “treatment”, as used herein, is defined as the application or administration of a therapeutic agent to a patient, or application or administration of a therapeutic agent to an isolated tissue or cell line from a patient, who has a disease, disorder or condition, a symptom of a disease, disorder or condition or a predisposition toward a disease, disorder or condition, with the purpose of preventing, curing, healing, alleviating, relieving, altering, remedying, ameliorating, improving or affecting the disease or disorder, the symptoms of disease or disorder or the predisposition toward a disease or disorder.

The term “subject” is intended to include animals, which are capable of suffering from or afflicted with a gated ion channel-associated state or gated ion channel associated disease, disorder, or condition, or any disease, disorder, or condition involving, directly or indirectly, gated ion channel activity. Examples of subjects include mammals, e.g., humans, dogs, cows, horses, pigs, sheep, goats, cats, mice, rabbits, rats, and transgenic non-human animals. In certain embodiments, the subject is a human, e.g., a human suffering from, at risk of suffering from, or potentially capable of suffering from a gated ion channel-associated state or gated ion channel-associated disease, disorder, or condition.

Examples of gated ion channel-associated diseases, disorders and conditions include, but are not limited to, pain (e.g, inflammatory pain, acute pain, chronic malignant pain, chronic nonmalignant pain, visceral pain, neuropathic pain and migraine), inflammatory disorders and neurological disorders (e.g., neurodegenerative or neuropsychiatric disorders).

The term “pain” as used herein refers to a sensation of discomfort that can be described as “unpleasant sensory and emotional experience associated with actual or potential tissue damage, or described in terms of such damage” (as defined by the International Association for the Study of Pain (IASP)). Pain can range from mild, localized discomfort to agony and results from the stimulation (e.g, via ASIC activity) of specialized nociceptive neurons. Pain is generally associated with tissue damage or inflammation. “Nociception” is the activity by which the nervous system detects noxious, i.e. potentially tissue-damaging, stimuli (also referred to as physiological pain). Pain can be subdivided into nociceptive pain (defined as pain caused by damage to tissues and characterized by the ongoing activity of Aδ and C-nociceptors) and neuropathic pain (defined as pain caused by the aberrant signal processing in the nervous system due to nervous system injury, damage or impairment). Pain can be further grouped into three categories based on the anatomical localization, including cutaneous pain, somatic (or deep somatic) pain and visceral pain. (It is recognized that certain disorders are associated with more than one category of pain.)

“Cutaneous pain” is caused by injury to the skin or superficial tissues. Cutaneous nociceptors terminate just below the skin, and due to the high concentration of nerve endings, produce a well-defined, localized pain of short duration. Examples of injuries that produce cutaneous pain include, but are not limited to, cuts, burns and lacerations, as well as traumatic injury and post-operative or surgical pain(e.g., at the site of incision).

“Somatic pain” originates from injury, inflammation or disease of the ligaments, tendons, bones, blood vessels, and nerves themselves, and is detected with somatic nociceptors. The scarcity of pain receptors in these areas produces a dull, poorly-localized pain of longer duration than cutaneous pain. Examples of somatic pain include, but are not limited to, sprains, broken bones, arthralgia, vasculitis, myalgia and myofascial pain. Arthralgia refers to pain caused by a joint that has been injured (such as a contusion, break or dislocation) and/or inflamed (e.g., arthritis). Vaculitis refers to inflammation of blood vessels with pain. Myalgia refers to pain originating from the muscles. Myofascial pain refers to pain stemming from injury or inflammation of the fascia and/or muscles. Somatic pain may also be associated with diseases or disorders of the ligaments, tendons, bones, blood vessels and nerves, including, but not limited to, disorders of the musculoskeletal system and connective tissues, and disorders of the circulatory system,

“Visceral” pain is associated with injury, inflammation or disease of the body organs and internal cavities. Disorders that are associated with visceral pain include, but are not limited, to disorders of the circulatory system, respiratory system, gastrointestinal system, genitourinary system, immune system, as well as ear, nose and throat. Visceral pain can also be associated with infectious and parasitic diseases that affect the body organs and tissues. The even greater scarcity of nociceptors in body organs and cavities produces a pain usually more aching and of a longer duration than somatic pain. Visceral pain is extremely difficult to localize, and several injuries to visceral tissue exhibit “referred” pain, where the sensation is localized to an area completely unrelated to the site of injury. For example, myocardial ischaemia (the loss of blood flow to a part of the heart muscle tissue) is possibly the best known example of referred pain; the sensation can occur in the upper chest as a restricted feeling, or as an ache in the left shoulder, arm or even hand. Phantom limb pain is the sensation of pain from a limb that one no longer has or no longer gets physical signals from—an experience almost universally reported by amputees and quadriplegics.

“Neuropathic pain” (“neuralgia”) can occur as a result of injury, inflammation or disease to the nerve tissue itself, for example, caused by a nerve or nerves that are irritated, trapped, pinched, severed or inflamed (neuritis). This can disrupt the ability of the sensory nerves to transmit correct information to the thalamus, and hence the brain interprets painful stimuli even though there is no obvious or documented physiologic cause for the pain. Disorders of the nerve tissue include, but are not limited to, disorders of the nervous system.

Pain can also be categorized as being acute or chronic. “Acute pain” is defined as short-term pain or pain with an easily identifiable cause. It is often fast and sharp and centralized to one area followed by aching pain that can be spread out. “Chronic pain” is defined as constant or intermittent pain that has lasted longer than the expected time of healing, e.g., at least about 2 weeks, 3 weeks, one month, two months, thee months, six months or longer.

As used herein the term “inflammatory disease or disorder” includes diseases or disorders which are caused, at least in part, or exacerbated by inflammation, e.g., increased blood flow, edema, activation of immune cells (e.g., proliferation, cytokine production, or enhanced phagocytosis). Inflammatory disorders are generally characterized by heat, redness, swelling, pain and loss of function. The cause of inflammation may be due to physical damage, chemical substances, micro-organisms, tissue necrosis, cancer or other agents. Inflammatory disorders include acute inflammatory disorders, chronic inflammatory disorders, inflammatory polyarthropathies (e.g., rheumatoid arthritis, juvenile arthritis, polyarthritis, gout), osteoarthritis, rheumatoid arthritis, spondyloarthropathies and recurrent inflammatory disorders. Acute inflammatory disorders are generally of relatively short duration, and last for from about a few minutes to about one to two days, although they may last several weeks. The main characteristics of acute inflammatory disorders include increased blood flow, exudation of fluid and plasma proteins (edema) and emigration of leukocytes, such as neutrophils. Chronic inflammatory disorders, generally, are of longer duration, e.g., weeks to months to years or longer, and are associated histologically with the presence of lymphocytes and macrophages and with proliferation of blood vessels and connective tissue. Recurrent inflammatory disorders include disorders which recur after a period of time or which have periodic episodes. Some disorders may fall within one or more categories.

Inflammatory disorders that may be treated according to the methods of the invention include, but are not limited to, inflammation of the nervous system, circulatory system, respiratory system, musculoskeletal and connective tissue system, gastrointestinal system, genitourinary system, eye and adnexa, ear, nose and throat, and endocrine system. Examples of causes of inflammatory disorders include, but are not limited to, microbial infections (e.g., bacterial, viral and fungal infections), physical agents (e.g., burns, radiation, and trauma), chemical agents (e.g., toxins and caustic substances), tissue necrosis and various types of immunologic reactions.

The terms “neurological disorder,” “neurodegenerative disorder” and “neuropsychiatric disorder” can be used interchangeably herein and refer to disorders and states (e.g., a disease state) that are also associated with gated ion channel-associated diseases, disorders and conditions. For example, a neurological disorder can be associated with inappropriate sympathetic or parasympathetic nerve function. Examples of neurological states that may be treated according to the methods of the invention include, but are not limited to schizophrenia, biopolar disorder, depression, Alzheimer's disease, epilepsy, cancer, musculoskeletal diseases, multiple sclerosis, amyotrophic lateral sclerosis, stroke, addiction, cerebral ischemia, cardiac disease (e.g., cardiac arrhythmia), neuropathy (e.g., anticancer-agent-intoxicated neuropathy, diabetic neuropathy), retinal pigment degeneration, glaucoma, Huntington's chorea, and Parkinson's disease. As used herein, “neuropathy” is defined as a failure of the nerves that carry information to and from the brain and spinal cord resulting in one or more of pain, loss of sensation, loss of function, and inability to control muscles. In some cases, the failure of nerves that control blood vessels, intestines, and other organs results in abnormal blood pressure, digestion problems, and loss of other basic body processes. Peripheral neuropathy may involve damage to a single nerve or nerve group (mononeuropathy) or may affect multiple nerves (polyneuropathy).

The above compounds can be used for administration to a subject for the modulation of a gated ion channel-mediated activity, including, but not limited to pain, inflammatory and neurological disorders, including disorders and states (e.g., a disease state) that are associated with gated ion channel-mediated activity, including, but not limited to, abnormal neuron growth, abnormal neuron proliferation or abnormal neuron function, abnormal neurotransmission and/or any abnormal function of cells, organs, or physiological systems that are modulated, at least in part, by a gated ion channel-mediated activity.

Accordingly, the compounds of the invention may be used to treat pain, and it is understood that the compounds may also alleviate or treat one or more additional symptoms of a disease or disorder discussed herein, e.g., inflammatory and/or neurological disorder.

Examples of pain that may be treated according to the methods of the invention include, but are not limited to pain associated with injury, trauma, cutaneous pain, somatic pain, visceral pain, neuropathic pain, nociceptive pain, acute pain, chronic malignant pain, chronic nonmalignant pain, post-operative pain, cancer pain and inflammatory pain.

Examples of cutaneous pain include, but are not limited to pain related to cuts, burns, lacerations, punctures, incisions, surgical pain, post-operative pain, including orodental surgery, and pain associated with inflammation and infection.

Examples of somatic pain include, but are not limited to arthralgia, myalgia, myofascial pain syndrome, chronic lower back pain, cancer-associated pain, phantom limb pain, central pain, bone injury pain, dental pain, fibromyalgia syndrome, meralgia paraesthetica, fibrocitis, idiopathic pain disorder, atypical odontalgia, loin pain, non-cardiac chest pain, chronic nonspecific pain, musculoskeletal pain disorder, chronic pelvic pain, and pain during labor and delivery, post-operative pain, cluster headaches, surgical pain, pain resulting from severe, for example third degree, burns, post partum pain, postmastectomy pain syndrome, stump pain, referred pain, reflex sympathetic dystrophy, and causalgia.

Examples of somatic pain further include, but are not limited to, pain related to injuries, diseases or disorders associated with the musculoskeletal system and connective tissues. Examples of musculoskeletal system and connective tissue injuries and disorders include, sprains, broken bones, arthropathies (e.g., various forms of arthritis, rheumatoid arthritis, osteoarthritis, spondyloarthropathies and gout), dorsopathies (e.g., various forms of scoliosis, kyphosis, lordosis, osteochondrosis, spondylolysis, subluxation, and torticollis), myositis and diseases of the muscles (e.g., infective myositis, interstitial myositis, calcification and ossification of muscle, diastasis of muscle, ischaemic infarction of muscle, and muscle strain), osteopathies and chondropathies (e.g., various forms of osteoporosis with or without pathological fracture including, but not limited to postmenopausal, drug-induced, and idiopathic osteoporosis.), osteomalacia, disorders of continuity of bone (e.g., malunion or nonunion of fracture, stress fracture, and pathological fracture), disorders of bone density and structure (e.g., fibrous dysplasia, skeletal fluorosis, osteitis and condensans) and disorders of the skin (e.g., psoriasis, eczema and dermatitis).

Additional examples of somatic pain include, but are not limited to, pain related to injuries, diseases or disorders associated with the circulatory system. Examples of circulatory system injuries and disorders include, but are not limited to, acute and chronic rheumatic heart diseases, myocardial infarction, hypertension and hypotension, rheumatic fever, pulmonary embolism, cerebrovascular diseases (e.g., stroke), atherosclerosis, peripheral vascular disease (e.g., intermittent claudication), hypertensive diseases, ischaemic heart diseases. including angina pectoris, acute myocardial infarction, coronary thrombosis, coronary insufficiency, mitral insufficiency, hypertrophic cardiomyopathy, ventricular cardiac arrhythmia (e.g., sustained ventricular tachydardia, non-sustained ventricular tachycardia, ventricular fibrillation, ventricular premature beats and ventricular flutter), and atrial tachyarrhythmia (e.g., atrial fibrillation and atrial flutter) and Dressler's syndrome; diseases of arteries, arterioles and capillaries (e.g., atherosclerosis, aneurysm, peripheral vascular disease, Raynaud's syndrome, Buerger, intermittent claudication, acrocyanosis and erythrocyanosis); diseases of veins, lymphatic vessels and lymph nodes (e.g., various forms of phlebitis, thrombophlebitis, embolism or thrombosis of the veins, varicose veins, haemorrhoids, varices, (including oesophageal, gastric, scrotal, pelvic), lymphadenitis, lymphangitis, and lymphoedema.

Disorders of the respiratory system associated with visceral pain include, but are not limited to acute and chronic upper respiratory infections (e.g., various forms or manifestations of acute or chronic nasopharyngitis, sinusitis, pharyngitis, tonsillitis, laryngitis, tracheitis, laryngotracheitis, rhinitis), cough, influenza, various forms of pneumoniae (e.g., bacterial, viral, parasitic or fungal), acute or chronic lower respiratory infections (e.g., various forms or manifestations of acute or chronic bronchitis, bronchiolitis, tracheobronchitis, emphysema, bronchiectasis, status asthmaticus, asthma and other chronic obstructive pulmonary diseases (COPD), adult respiratory distress syndrome, pulmonary oedema, pyothorax, and diseases of the pleura.

Disorders of the gastrointestinal system associated with visceral pain include, but are not limited to, disorders of the tooth (e.g., anodontia, supernumerary teeth, mottled teeth, teething syndrome, embedded and impacted teeth, dental caries, pathological resorption, ankylosis of teeth, hypercementosis, pulpitis, necrosis or degeneration of the pulp, various forms of acute or chronic gingivitis, periodontitis, or periodontal disease, gingival recession); dentofacial anomalies (e.g., manadibular hyper- or hypoplasia, asymmetry of jaw, retrognathism, crossbite, temporomandibular joint disorders), orodental cysts, inflammatory conditions of jaws, diseases of the salivary glands (e.g., sialoadenitis, sialolithiasis, abscess, fistula or mucocele of the salivary gland); diseases of the lip and oral mucosa (e.g., various forms of stomatitis, recurrent oral aphthae, cellulites and abscess of the mouth); diseases of the tongue (e.g., various forms of glossitis, glossodynia, hypertrophy of tongue papillae); diseases of the oesophagus, stomach and duodenum (e.g., oesophagitis, Gastro-oesophageal reflux disease, Achalasia of cardia, ulcer of oesophagus, dyskinesia of oesophagus, diverticulum of oesophagus, ulcers (e.g., oesophageal, gastric, duodenal, gastrojejunal); various forms and manifestations of gastritis and duodenitis, (e.g., dyspepsia, pyloric stenosis, pylorospasms), esophageal varicies and refractory ascites and esophageal carcinoma; diseases of the appendix (e.g., various forms and manifestations of appendicitis); hernia (e.g,. various forms and manifestations of inguinal, femoral, umbilical, ventral, or diaphragmatic hernia), diseases of the intestines including noninfective enteritis and colitis (e.g., various forms and manifestations of Crohn's disease, ulcerative colitis, collagenous colitis, gastroenteritis and colitis due to radiation, toxic, allergic or dietetic gastroenteritis and colitis, as well as various forms of ileitis, jejunitis or sigmoiditis), acute or chronic vascular disorders of the intestine (e.g., fulminant ischaemic colitis, intestinal infarction, chronic ischaemic colitis or enteritis, mesenteric vascular insufficiency, angiodysplasia of colon), paralytic ileus and intestinal obstruction (e.g., volvulus, gallstone ileus, intestinal occlusion), diverticular disease of intestine with or without perforation and/or abscess (e.g., diverticulitis, diverticulosis, diverticulum), irritable bowel syndrome, constipation, diarrhea (e.g., functional diarrhea, and infectious diarrhea such as diarrhea associated with amebiasis, giardiasis, viral infection, cytomegalovirus infection, or pathogenic bacterial infection), neurogenic bowel, megacolon, anal spasm (proctalgia fugax), fissure, fistula or abscess of anal and rectal regions, anal or rectal polyp, anal or rectal prolapse, anal or rectal stenosis, ulcer of anus or rectum, radiation proctitis), diseases of the peritoneum (e.g. various forms and manifestations of acute or chronic peritonitis, peritoneal adhesions, haemoperitoneum), diseases of the liver such as various forms and manifestations of alcoholic liver disease (e.g., alcoholic fatty liver, alcoholic hepatitis, alcoholic fibrosis and sclerosis of liver, alcoholic cirrhosis of liver, alcoholic hepatic failure), various forms and manifestations of acute, subacute or chronic hepatic failure, of acute and chronic hepatitis (e.g., nonspecific reactive hepatitis, autoimmune hepatitis, chronic persistent hepatitis, chronic lobular hepatitis, granulomatous hepatitis, infectious or parasitic hepatitis (e.g., cytomegaloviral, herpesviral, toxoplasma) of fibrosis and cirrhosis of liver (e.g.. hepatic fibrosis and/or sclerosis, cardiac sclerosis of liver, primary or secondary biliary cirrhosis, macronodular cirrhosis, cryptogenic cirrhosis), liver necrosis, infarction of liver, hepatic veno-occlusive disease, Budd-Chiari syndrome, portal hypertension, hepatorenal syndrome, focal nodular hyperplasia of liver, hepatoptosis, various forms and manifestations of toxic or idiosyncratic liver disease; disorders of gallbladder, biliary tract and pancreas (e.g., cholelithiasis, cholecystolithiasis, choledocholithiasis, gallstone or calculus of gallbladder with or without cholecystitis), gallstone or calculus of bile duct (with or without cholecystitis and/or cholangitis), acute or chronic cholecystitis (e.g., emphysematous, gangrenous or suppurative cholecystitis, empyema or gangrene of gallbladder), occlusion, stenosis or stricture of cystic duct or gallbladder without calculus, hydrops, perforation or fistula of gallbladder, cholecystocolic or cholecystoduodenal fistula, cholesterolosis of gallbladder, cholangitis (e.g., ascending, primary, secondary, recurrent, sclerosing, or stenosing cholangitis), obstruction of the bile duct without calculus, spasm of sphincter of Oddi, biary cyst, various forms and manifestations of pancreatitis (e.g., acute, subacute or chronic pancreatitis, infectious pancraetitis, haemorrhagic pancreatitis, suppurative pancreatitis), pancreatic steatorrhoea, cyst or pseudocyst of the pancreas, coeliac disease, gluten-sensitive enteropathy, idiopathic steatorrhoea, gastric or intestinal haemorrhage, functional abdominal pain syndrome (FAPS), gastrointestinal motility disorders, faecal incontinence, and various forms of cancer and neoplasm of the gastrointestinal system (e.g., neoplasm of the oesophagus, stomach, small intestine, colon, liver and pancreas) Disorders of the genitourinary system associated with visceral pain include, but are not limited to, glomerular diseases nephritic syndromes (e.g., glomerulonephritis, nephritis, acute, infectious or chronic tubulo-interstitial nephritis, diffuse sclerosing glomerulonephritis, recurrent and persistent haematuria), nephrotic syndrome, lipoid nephrosis, proteinuria (e.g., Bence Jones, gestational, orthostatic, persistent), glomerular disorders in other diseases (e.g., infectious and parasitic diseases, blood diseases and disorders involving the immune mechanism, diabetes mellitus, systemic connective tissue disorders, endocrine, nutritional and metabolic diseases)], acute or chronic renal tubulo-interstitial diseases (e.g., interstitial nephritis, infectious interstitial nephritis, pyelitis, pyelonephritis), chronic obstructive or non-obstructive pyelonephritis, obstructive and reflux uropathy, nephropathy (e.g., analgesic nephropathy, nephrpathy induced by drugs, medicaments, heavy metals and biological substances, renal tubulo-interstitial disorders in other diseases (e.g., in infectious and parasitic diseases, in neoplastic diseases, in blood diseases and disorders involving the immune, in metabolic diseases, in systemic connective tissue disorders, or in transplant rejection), acute or chronic renal failure (with tubular, cortical or medullary necrosis) uraemia, urolithiasis (e.g., calculus of kidney and/or ureter, nephrolithiasis, renal calculus or stone, Staghom calculus, ureteric stone, calculous pyelonephritis), calculus of lower urinary tract, bladder or urethra, renal colic), renal osteodystrophy, azotaemic osteodystrophy, nephrogenic diabetes insipidus, Lightwood-Albright syndrome, renal tubular acidosis, atrophy or hypertrophy of kidney, unilateral or bilateral Ischaemia and infarction of kidney, megaloureter, nephroptosis, pyelitis, pyeloureteritis, ureteritis, ureterocele, disorders of kidney and ureter in infectious and parasitic diseases (e.g., schistosomiasis, tuberculosis, syphilis), polycystic kidney disease, cystitis (e.g., acute cystitis, prostatocystitis, interstitial cystitis, irradiation cystitis, trigonitis, urethrotrigonitis) uninhibited, reflex or flaccid neuropathic bladder, neuromuscular dysfunction of bladder, neurogenic bladder dysfunction, overactive bladder, Bladder-neck obstruction (e.g., bladder-neck stenosis, vesicointestinal fistula, vesical fistula, diverticulum of bladder), bladder disorders in diseases (e.g., tuberculous cystitis, balder disorder in schistosomiasis), frequent micturition, polyuria, oliguria, anuria, nocturia, enuresis, dysuria, urinary incontinence, pneumaturia, disorders of urethra (e.g. urethritis and urethral syndrome, ulcer of urethra, urethral meatitis, urethral stricture, urethral, urethroperineal or urethrorectal fistula, urethral diverticulum, urethral caruncle, prolapsed urethral mucosa, urethritis and urethral disorders in other diseases such as candidal urethritis), urinary tract infection, urinary incontinence (overflow, reflex or urge), stress incontinence, diseases of the male genital organs (e.g., hyperplasia of prostate including adenofibromatous hypertrophy, adenoma, fibroadenoma, fibroma, hypertrophy, myoma), inflammatory diseases of prostate (e.g., acute or chronic prostatitis, abscesses of the prostate, prostatocystitis), calculus of prostate, congestion and haemorrhage of prostate, atrophy of prostate, hydrocele (e.g., hydrocele of spermatic cord, testis, or tunica vaginalis, encysted hydrocele, infected hydrocele, spermatocele), Torsion of testis, orchitis, epididymitis and epididymo-orhitis with or without abscess, redundant prepuce, phimosis and paraphimosis, leukoplakia of penis (e.g., Balanitis xerotica obliterans, Kraurosis pf penis), Balanoposthitis, priapism, ulcer of penis, induratio penis plastica (e.g., Peyrone's disese), atrophy, hypertrophy or thrombosis of corpus cavemosum and penis, inflammatory disorders of seminal vesicle (e.g., vesiculitis), of spermatic cord, of tunica vaginalis, of vas deferens, or of scrotum, atrophy of testis, vascular disorders of male genital organs (e.g, haematocele, haemorrhage, thrombosis), disorders of male genital organs in other diseases (e.g., gonococal, trichonal or tuberculous prostatitis, chlamydial, gonoccocal, or tuberculous epididymitis and/or orchitis, filarial chylocele, herpesviral infection of genital tract, tuberculosis of seminal vesicle), disorders of breast [e.g. benign mammary dysplasia, fibrocystic or diffuse mastopathy, cyst of the breast, fibroadenosis or fibrosclerosis of the breast, inflammatory disorders of the breast (e.g. abscess, carbuncle, acute, subacute or nonpurpuerperal mastitis), hypertrophy, lump in breast, fissure and fistula of nipple, fat necrosis and atrophy of the breast, galactorrhea, mastodynia, induration of breast, galactocele, inflammatory diseases of female pelvis organs (e.g., acute or chronic salpingitis and oophoritis), (abscess of fallopian tubes and/or ovary, pyosalpinx, salpingo-oophoritis, hydrosalpinx), acute and chronic inflammatory diseases of the uterus and cervix (e.g., endomyometritis, metritis, myometritis, pyometra, uterine abscess, cervicitis, endocervicitis, exocervicitis), acute and chronic parametritis and pelvic cellulites, acute and chronic pelvic peritonitis, female pelvic inflammatory disorders associated with other diseases (e.g., tuberculous infection of the cervix uteri, syphilitic, gonococcal and chlamydial pelvic inflammatory disorders), diseases of Bartholin's gland, inflammation of the vagina and vulva (e.g., acute, subacute and chronic vaginitis, acute, subacute and chronic vulvitis, vulvovaginitis as well as vaginitis, vulvitis and vulvovaginitis in infectious and parasitic diseases (eg., candidiasis, herpesviral infection and pinworm infection), ulceration of vagina and vulva (e.g., ulceration in hepesviral infection and tuberculosis), endometriosis ( e.g., endometriosis of the uterus, ovary, fallopian tube, pelvic peritoneum, vagina and intestine), female genital prolapse, fistulae involving female genital tract, polyp (e.g., polyp of corpus uteri, cervix uteri, vagina and vulva), dysplasia of the cervix uteri, vagina and vulva, menstruation disorders (e.g., primary and secondary amenorrhea, oligomenorrhea, excessive menstruation, ovulation bleeding, menorrhagia), pain associated with female genital organs and menstrual cycle (e.g., Mittleschmerz, dyspareunia, vaginismus, primary and secondary dysmenorrheal and postmenopausal disorders), neoplasm of the genitourinary system (e.g., neoplasm of the kidney, urether, urethra, bladder, cervix, uterus, vagina, vulva, ovary, penis, prostate and testis) Examples of diseases or disorders associated with neuropathic pain include, but are not limited to, neuralgia (e.g., posttherapeutic neuralgia, postherpetic neuralgia and trigeminal neuralgia), neuropathy (e.g., diabetic neuropathy), neuropathic pain, orofacial neuropathic pain, pain associated with cancer, psychogenic pain, headache (e.g., nonorganic chronic headache, tension-type headache, cluster headache and migraine), conditions associated with chronic cephalic pain, complex regional pain syndrome, nerve trunk pain, somatoform pain disorder, cyclical mastalgia, chronic fatigue syndrome, multiple somatization syndrome, chronic pain disorder, tabes dorsalis, spinal cord injury, central pain, noncardiac chest pain, central post-stroke pain, shingles, and Morton's neuroma.

Inflammatory disorders that may be treated according to the methods of the invention include, but are not limited to, inflammation associated with microbial infections (e.g., bacterial, viral and fungal infections), physical agents (e.g., burns, radiation, and trauma), chemical agents (e.g., toxins and caustic substances), tissue necrosis and various types of immunologic reactions. Examples of inflammatory disorders further include, but are not limited to, disorders of the musculoskeletal and connective tissue system, disorders of the respiratory system, disorders of the circulatory system, disorders of the genitourinary system and disorders of the gastrointestinal system. Inflammatory disorders of these systems include, but are not limited to those exemplified above. Exemplary inflammatory disorders include, but are not limited to arthritis (e.g., osteoarthritis, rheumatoid arthritis), acute and chronic infections (bacterial, viral and fungal); acute and chronic bronchitis, sinusitis, and other respiratory infections, including the common cold; acute and chronic asthma; acute and chronic gastroenteritis and colitis; acute and chronic cystitis and urethritis; acute respiratory distress syndrome; cystic fibrosis; acute and chronic dermatitis; acute and chronic conjunctivitis; acute and chronic serositis (pericarditis, peritonitis, synovitis, pleuritis and tendinitis); uremic pericarditis; acute and chronic cholecystis; acute and chronic vaginitis; acute and chronic uveitis; lupus erythematosus, eczema, shingles, psoriasis, hyperalgesia, irritable bowl syndrome, Crohn's disease, multiple sclerosis, drug reactions; and burns (thermal, chemical, and electrical).

Neurological disorders that may be treated according to the methods of the invention include, but are not limited to schizophrenia, schizotypal disorders, schizoaffective disorders (e.g., manic type, depressive type, and mixed type), various forms and manifestations of dementia and delirium, delusional disorders, psychotic disorders (e.g., transient acute psychotic disorders, acute polymorphic psychotic disorders with or without symptoms of schizophrenia), bipolar affective disorder, cyclothymia, dysthemia, manic episodes, hypomania, depression, (e.g., mild, moderate and severe depressive episodes, psychotic depression, recurrent depressive disorder with and without psychotic symptoms, atypical depression), Anxiety disorders (e.g., phobic disorders (e.g., agoraphobia, claustrophobia), panic disorders, phobias, anxiety hyteria, generalized anxiety disorder, and neurosis), obsessive-compulsive disorders, acute stress reaction, post-traumatic stress disorder, dissociative disorders (e.g., dissociative amnesia, dissociative fugue, dissociative stupor, dissociative convulsions, dissociative motor disorders, trance, and possession disorders,) somatoform disorders (e.g., somatization disorder, persistent somatoform pain disorder, and somatoform autonomic dysfunction, psychogenic dysmenorrhoea, teeth-grinding), neurasthenia, eating disorders (e.g., anorexia nervosa, bulimia nervosa, psychogenic overeating, and psychogenic vomiting), sleep disorders (e.g., insomnia, hypersomnia, somnambulism, sleep terrors, and nightmares), nonorganic sexual dysfunction, learning disabilities and disorders, extrapyramidal and movement disorders (e.g., Parkinson's disease, secondary parkinsonism (e.g., drug-induced, malignant neuroleptic syndrome), degenerative diseases of the basal ganglia (e.g., Hallervorden-Spatz disease, supranuclear ophtalmoplegia, and striatonigral degeneration), dystonia (e.g., drug-induced, idiopathic familial dystonia, orofacial dystonia, spasmodic torticollis, and blepharospasm), tremor, myoclonus, chorea, tics, restless legs syndrome, and stiff-man syndrome), degenerative diseases, (e.g., Alzheimer's disease, Reye's syndrome, Pick's disease, progressive isolated aphasia, Alper's disease, Leigh's disease), demyelinating diseases (e.g., multiple sclerosis, diffuse sclerosis (e.g., Schilder's diseases), concentric sclerosis (e.g., Balo disease), neuromyelitis optica, acute disseminated demyelination (e.g., Hurt's disease), central pontine myelinolysis, and necrosing myelitis), various forms of epilepsy and epileptic syndromes (e.g., seizures, Grand mal, Petit mal, Landau-Kleffner syndrome, Todd's paralysis and status epilepticus, various forms of migraines and headaches (e.g., migraines with or without aura, status migrainosus, cluster headache syndrome, chronic paroxysmal hemicrania, tension-type headaches, and chronic post-traumatic headaches), transient cerebral ischaemic attacks (e.g, vertebro-basilar artery syndrome, carotid artery syndrome, amaurosis fugax, and transient global amnesia), vascular syndromes of brain (e.g., cerebral artery syndrome (e.g., middle, anterior and posterior artery syndrome), brain stem stroke syndrome (e.g. Benedikt, Claude, Fovillle, Millard-Gubler, Wallenberg and Weber syndromes), cerebellar stroke syndrome, lacunar syndromes (e.g., pure motor and pure sensory lacunar syndromes), sleep disorders (e.g., insomnias, hypersomnias, delayed sleep wake syndrome, sleep apnoea, narcolepsy, cataplexy, Kleine-Levin syndrome, and pickwickian syndrome), disorders of the trigeminal nerve (e.g., trigeminal neuralgia and atypical facial pain), facial and other cranial nerve disorders (e.g., Bell's palsy, geniculate ganglionitis, Melkersson's syndrome, clonic hemifacial spasm, facial myokymia, glossopharyngeal neuralgia, polyneuritis cranialis, postzoster neuralgia, and multiple cranial nerve palsies), nerve root and plexus disorders (e.g., thoracic outlet syndrome, neuralgic amyotrophy (e.g., Parsonage-Aldren-Turner syndrome, shoulder-girdle neuritis) and phantom limb syndrome with and without pain), mononeuropathies (e.g., carpal tunnel syndrome, Tardy ulnar nerve syndrome, causalgia, interdigital neuroma, sciatica, meralgia paraesthetica, tarsal tunnel syndrome, Morton's metatarsalgia, peroneal nerve palsy, intercostal neuropathy, mononeuritis multiplex, and diabetic mononeuropathy), hereditary and iodopathic polyneuropathies (e.g., Charcot-Marie-Tooth disease, Déjerine-Sottas disease, peroneal muscular atrophy, Roussy-Levy syndrome, Refsum's disease, Morvan's disease, and Nelaton's syndrome), inflammatory and other polyneuropathies (e.g., Guillain-Barré syndrome, drug-induced, radiation-induced and alcoholic polyneuropathies, diabetic polyneuropathies, polyneuropathies in infectious and parasitic diseases, in neoplastic diseases, in nutritional deficiency, in musculoskeletal disorders and in systemic connective tissue disorders), myoneural disorders (e.g., myasthenia gravis, congenital myasthenia, toxic myoneural disorders, various forms muscular dystrophy, myotonic disorders (e.g., myotonia, chondrodystrophic myotonia, myotonia congenita, paramyotonia congenita, pseudomyotonia), other myopathies (e.g., congenital, alcoholic, drug-induced, and inflammatory myopathies), myasthenic syndromes in other diseases (e.g., infectious and parasitic diseases, endocrine diseases, diabetic amyotrophy, neoplastic diseases, in metabolic diseases, rheumatoid arthritis, scleroderma, sicca syndrome, and sytemic lupus erythematosus), Eaton-Lambert syndrome), various forms and manifestations of cerebral palsy, hemiplegia, paraplegia, tetraplegia, diplegia and monoplegia, and cauda equina syndrome), disorders of the autonomic nervous system (e.g., familial dysautonomia, Riley-Day, Horner's disease, Shy-Drager syndrome, and carotid sinus syncope), cerebral cyst, anoxic brain damage, cerebral oedema, hydrocephalus, musculoskeletal diseases, neuromuscular diseases (e.g., muscular dystrophy), amyotrophic lateral sclerosis, systemic atrophies (e.g., Huntingdon's chorea, ataxia (e.g., hereditary ataxia, congenital ataxia, cerebellar ataxia), hereditary spastic paraplegia, spinal muscular atrophy, motor neuron disease, paraneoplastic neuromyopathy and neuropathy), stroke, addiction, cerebral ischemia, ventricular cardiac arrhythmia (e.g., sustained ventricular tachydardia, non-sustained ventricular tachycardia, ventricular fibrillation, ventricular premature beats and ventricular flutter), and atrial tachyarrhythmia (e.g., atrial fibrillation and atrial flutter), neuropathy (e.g., anticancer-agent-intoxicated neuropathy, diabetic neuropathy), retinal pigment degeneration, glaucoma, Huntington's chorea, Parkinson's disease diseases of the eye (e.g., claucoma, chorioretinal inflammation, chroidal degeneration, retinal vascular occlusions, peripheral retinal degeneration, retinopathies (e.g. diabetic retinopathy, atherosclerotic retinopathy), and optic neuropathies), and cancer of the nervous system (e.g., brain and spinal cord).

The methods of the present invention also include the treatment of a disease, disorder or condition that would benefit from the modulation of a gated ion channel activity in combination with an adjuvant composition, such as for example other pain drugs. As used herein the phrase, “pain drugs” is intended to refer to analgesics, anti-inflammatory agents, anesthetics, corticosteroids, antiepileptics, barbiturates, antidepressants, and marijuana.

As used herein, an “analgesic” is an agent that relieves pain without significant impairment of consciousness or sense perception and may result in the reduction of inflammation as do corticosteroids, e.g., an anti-inflammatory agent. Analgesics can be subdivided into NSAIDs (non-steroidal-anti-inflammatory agents) narcotic analgesics, and non-narcotic agents. NSAIDs can be further subdivided into non-selective COX (cyclooxygenase) inhibitors, and selective COX2 inhibitors. Opioid analgesics can be natural, synthetic or semi-synthetic opioid (narcotic) analgesics, and include for example, morphine, codeine, meperidine, propxyphen, oxycodone, hydromorphone, heroine, tranadol, and fentanyl. Non-opioid analgesics (non-narcotic) analgesics include, for example, acetaminophen, paracetamol, clonidine, NMDA antagonists, and cannabinoids. Non-selective COX inhibitors include, but are not limited to acetylsalicylic acid (ASA), ibuprofen, naproxen, ketoprofen, piroxicam, etodolac, and bromfenac. Selective COX2 inhibitors include, but are not limited to celecoxib, valdecoxib, parecoxib, and etoricoxib.

As used herein an “anesthetic” is an agent that interferes with sense perception near the site of administration, a local anesthetic, or result in alteration or loss of consciousness, e.g., systemic anesthetic agents. Local anesthetics include but are not limited to lidocaine and buvicaine.

Non-limiting examples of antiepileptic agents are carbamazepine, phenytoin and gabapentin. Non-limiting examples of antidepressants are amitriptyline and desmethylimiprimine.

IV. Methods of Administration

The gated ion channel modulators, e.g., compounds of the invention, (also referred to herein as “active compounds”) of the invention can be incorporated into pharmaceutical compositions suitable for administration. Such compositions typically comprise the gated ion channel modulator and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., a compound of the invention, e.g., a gated ion channel modulator) in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. “Dosage unit form” as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit large therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

As defined herein, a therapeutically effective amount of active compound (i.e., an effective dosage) ranges from about 0.001 to 30 mg/kg body weight, preferably about 0.01 to 25 mg/kg body weight, more preferably about 0.1 to 20 mg/kg body weight, and even more preferably about 1 to 10 mg/kg, 2 to 9 mg/kg, 3 to 8 mg/kg, 4 to 7 mg/kg, or 5 to 6 mg/kg body weight. The skilled artisan will appreciate that certain factors may influence the dosage required to effectively treat a subject, including but not limited to the severity of the disease, disorder or condition, previous treatments, the general health and/or age of the subject, and other diseases, disorders or conditions present. Moreover, treatment of a subject with a therapeutically effective amount of compound of the invention can include a single treatment or, preferably, can include a series of treatments.

The pharmaceutical compositions can be included in a container, pack, or dispenser together with instructions for administration.

This invention is further illustrated by the following Exemplification which should not be construed as limiting. The contents of all references, sequences, Figures, and published patent applications cited throughout this application are hereby incorporated by reference.

Equivalents

Those skilled in the art will recognize, or be able to ascertain using no more than routine experimentation, many equivalents to the specific embodiments of the invention described herein. Such equivalents are intended to be encompassed by the following claims.

### INCORPORATION BY REFERENCE

The entire contents of all patents, published patent applications and other references cited herein are hereby expressly incorporated herein in their entireties by reference.

### EXAMPLES

### Example 1

### Screening and Bioanalysis of ASIC Antagonists

This example describes the in vitro assessment of the activity of the compounds of the present invention.

Electrophysiological assays in Xenopus laevis oocytes expressing gated ion channels are performed as follows:

Surgically removed oocytes from adult Xenopus laevis are treated for 2 h at room temperature with 1 mg/ml type I collagenase (Sigma) in Barth solution under constant agitation. Selected oocytes at stage IV-V are defolliculated manually before nuclear microinjection of 2.5-5 ng of a suitable expression vector, such as pCDNA3, comprising the nucleotide sequence encoding a gated ion channel subunit protein. In such an experiment, the oocytes express homomultimeric proton-gated ion channels on their surface. In an alternate experiment one, two, three or more vectors comprising the coding sequences for distinct gated ion channel subunits are co-injected in the oocyte nuclei. In the latter case, oocytes express heteromultimeric proton-gated ion channels. For example, ASIC2a and/or ASIC3 subunits in pcDNA3 vector are co-injected at a 1:1 cDNA ratio. After 2-4 days of expression at 19° C. in Barth solution containing 50 mg/ml gentamicin and 1.8 mM CaCl2, gated ion channels are activated by applying an acidic solution (pH<7) and currents are recorded in a two electrode voltage-clamp configuration, using an OC-725B amplifier (Warner Instruments). Currents are acquired and digitized at 500 Hz on an Apple Imac G3 computer with an AID NB-MIO-16XL interface (National Instruments) then recorded traces are post-filtered at 100 Hz in Axograph (Axon Instruments) (Neher, E. and Sakmann, B. (1976) Nature 260:799-802). Compounds of the present invention are prepared in a modified Ringer's solution containing 97 mM NaCl, 2 mM KCl, 1.8 mM CaCl2, 10 mM acetic acid, 10 mM Hepes (Sigma) brought to pH 7.3 with NaOH. Test solutions are prepared by lowering the pH with HCl and applied to oocytes by constant perfusion (10-12 ml/min) at room temperature. Osmolarity of all solutions is adjusted to 235 mOsm with choline chloride.

### Example 2

### Screening and Bioanalysis of ASIC Antagonists

This example describes another in vitro assessment of the activity of the compounds of the present invention.

Another example of an in vitro assessment method consists of using mammalian heterologous expression systems, which are known to the skilled in the art, and include a variety of mammalian cell lines such as COS, HEK, e.g., HEK293and/or CHO, cells. Cell lines are transfected with gated ion channel(s) and used to perform electrophysiology as follows:

All experiments are performed at room temperature (20-25° C.) in voltage clamp using conventional whole cell patch clamp methods (Neher, E., et al. (1978) Pfluegers Arch 375:219-228).

The amplifier used is the EPC-9 (HEKA-electronics, Lambrect, Germany) run by a Macintosh G3 computer via an ITC-16 interface. Experimental conditions are set with the Pulse-software accompanying the amplifier. Data is low pass filtered and sampled directly to hard-disk at a rate of 3 times the cut-off frequency.

Pipettes are pulled from borosilicate glass using a horizontal electrode puller (Zeitz-lnstrumente, Augsburg, Germany). The pipette resistances are 2-3 MOhms in the salt solutions used in these experiments. The pipette electrode is a chloridized silver wire, and the reference is a silver chloride pellet electrode (In Vivo Metric, Healdsburg, USA) fixed to the experimental chamber. The electrodes are zeroed with the open pipette in the bath just prior to sealing.

Coverslips with the cells are transferred to a 15 μl experimental chamber mounted on the stage of an inverted microscope (IMT-2, Olympus) supplied with Nomarski optics. Cells are continuously superfused with extracellular saline at a rate of 2.5 ml/min. After giga-seal formation, the whole cell configuration is attained by suction. The cells are held at a holding voltage of −60 mV and at the start of each experiment the current is continuously measured for 45 s to ensure a stable baseline. Solutions of low pH (<7) are delivered to the chamber through a custom-made gravity-driven flowpipe, the tip of which is placed approximately 50 μm from the cell. Application is triggered when the tubing connected to the flowpipe is compressed by a valve controlled by the Pulse-software. Initially, low pH (in general, pH 6.5) is applied for 1 s every 45 s. The sample interval during application is 550 μs. After stable responses are obtained, the extracellular saline as well as the low-pH solution are switched to solutions containing the compound to be tested. The compound is present until responses of a repeatable amplitude are achieved. Current amplitudes are measured at the peak of the responses, and effect of the compounds is calculated as the amplitude at compound equilibrium divided by the amplitude of the current evoked by the pulse just before the compound was included.

The following salt solutions are used: extracellular solution (mM): NaCl (140), KCl (4), CaCl2 (2), MgCl2 (4), HEPES (10, pH 7.4); intracellular solution (mM): KCl (120), KOH (31), MgCl2 (1.785), BGTA (10), HEPES (10, pH 7.2). In general, compounds for testing are dissolved in 50% DMSO at 500 fold the highest concentration used.

Results of the in vitro electrophysiology studies for effect on ASIC1a (HEK cells) are shown in Table 1 below:

TABLE 1

| Compound | IC₅₀ |
|---|---|
| 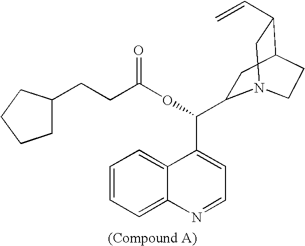 | IC₅₀: between 17 and 23 μM (ASIC1a) |
| (Compound A) | IC₅₀: between 4 and 6 μM (ASIC3) |

The data in Table 2 and Table 3 below demonstrate the results of pH dependency studies of Compound A (10 μM) with ASIC1a and ASIC3.

TABLE 2
ASIC1a - pH Dependency

| pH | Fraction of Control |
|---|---|
| 5.0 | greater than .85 |
| 6.0 | greater than .85 |
| 6.5 | between .70 and .85 |
| 6.8 | between .10 and .25 |

TABLE 3
ASIC3 - pH Dependency

| pH | Fraction of Control |
|---|---|
| 5.0 | greater than .75 |
| 6.0 | between .60 and .75 |
| 6.5 | between .30 and .45 |
| 6.8 | between .20 and .35 |

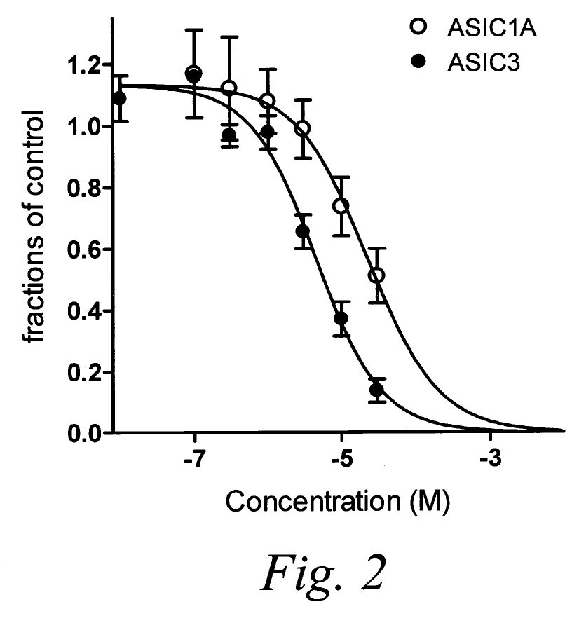

FIG. 2 demonstrates concentration response relationships between Compound A and ASIC1a and ASIC3 that were acquired at pH 6.5, using the patch-clamp procedure described in this example.

The patch-claim experiments described in this example demonstated that use of pentanoic acid (5-ethyl-1-aza-bicyclo[2.2.2]oct-2-yl)-(6-methoxy-quinolin-4-yl)-methyl ester and 3,3-dimethyl-butyric acid (5-ethyl-1-aza-bicyclo[2.2.2]oct-2-yl)-(6-methoxy-quinolin-4-yl)-methyl ester resulted in a larger difference in current between control ASICs and ASICs with 1 μM compound compared with pentanoic acid (6-methoxy-quinolin-4-yl)-(5-vinyl-1-aza-bicyclo[2.2.2]oct-2-yl)-methyl ester and 3,3-dimethyl-butyric acid (6-methoxy-quinolin-4-yl)-(5-vinyl-1-aza-bicyclo[2.2.2]oct-2-yl)-methyl ester did. These experiments were performed on ASIC3 and ASIC1a, and at pH 6.5 and pH 5.0.

### Example 3

### Screening and Bioanalysis of ASIC Antagonists

This example describes another in vitro assessment of the inhibitory activity of the compounds of the present invention utilizing patch-clamp electrophysiology of sensory neurons.

Sensory neurons can be isolated and cultured in vitro from different animal species. The most widely used protocols use sensory neurons isolated from neonatal (Eckert, et al. (1997) J Neurosci Methods 77:183-190) and embryonic (Vasko, et al. (1994) J Neurosci 14:4987-4997) rat. Trigeminal and dorsal root ganglion sensory neurons in culture exhibit certain characteristics of sensory neurons in vivo. Electrophysiology is performed as described above in Example 2.

### Example 4

### In Vivo Screening and Bioanalysis of ASIC Antagonists

This example describes the in vivo assessment of the inhibitory activity of the compounds of the present invention.

A number of well-established models of pain are described in the literature and are known to the skilled in the art (see, for example, Table 5). This example describes the use of the Formalin test.

Male Sprague-Dawley rats (body weight, 180-300 g) are housed together in groups of four animals under standard conditions with unrestricted access to food and water. Rats are housed in the room in which the testing procedure is performed to minimize any stress response to novel environmental cues. All experiments are conducted according to the ethical guidelines for investigations of experimental pain in conscious animals (Zimmerman, 1983).

Separate groups of rats are used for the different experiments such that each animal is used on one occasion only. All experiments for a given compound are performed using age matched animals in an attempt to avoid variability between experiments. Prior to initiation of the formalin test, 4 rats are habituated in separate testing cages for 15-20 min. The testing cages are constructed from open mesh steel (L29×H16×W22 cm), with angled mirrors placed underneath and behind the cages to allow for an unimpeded view of the animals paws. Animals are then given an injection of either compound or vehicle as appropriate for the testing paradigm. After 4-5 min, they are gently restrained and formalin (5%, 50 ml, s.c.; Formaldehyde solution minimum 37%, diluted 1:20 in saline) is injected into the plantar surface of the hindpaw using a 27 gauge needle. After injection, each rat is placed back in its testing cage and measurement of flinching behavior is initiated immediately by a skilled observer blinded to drug treatment. On the basis of the response pattern, two distinct phases of nociceptive behavior are observed, characterized by flinching of the affected paw and scored (Blackburn-Munro et al., Eur J Pharmacol. 2002 Jun. 12;445(3):231-8). The first phase, occurs on average 0-5 min after injection of formalin and the second phase occurs 15-40 min after the injection. Each flinch is registered on-line by the observer into a DOS-based PC program. Each rat is observed for 15 s in sequence and the 15 s bins are collated for each rat to obtain 5 min data bins for the 60 min duration of the experiment.

All data is analysed using Sigmastat 2.03 statistical software (Jandel Scientific GmbH, Germany) and expressed as mean±S.E.M. Two Way Repeated Measures Analysis of Variance (Two Way RM ANOVA) is used to analyse formalin test data and where applicable, this is followed by post-hoc analysis using Bonferroni's t-test to allow comparisons between groups. Comparisons of the total number of flinches recorded during the second phase of the formalin test between groups is made using One Way ANOVA. P<0.05 is considered statistically significant in all cases.

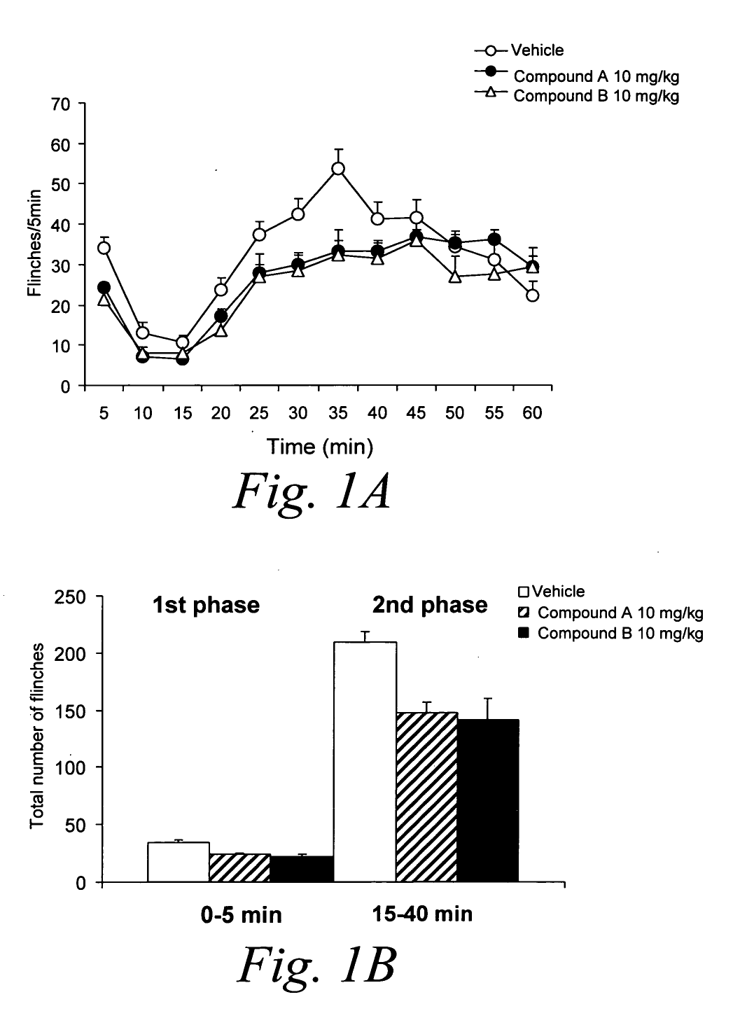

Results from the Formalin tests described in this example are demonstrated in FIGS. 1A and 1B. Compounds A and B (both 10 mg/kg in 0.9% NaCl) were injected i.v. 10 min prior to injection of formalin in the hind limb. Compounds A and B both attenuated flinching behaviour compared with injection of vehicle (isotonic glucose). All groups n=8 animals.

### Example 5

### Identification of ASIC Anatagonists using FLIPR

Cell Culture

ASIC1a expressing HEK293 cells are grown in culture medium (DMEM with 10% FBS), in polystyrene culture flasks (175 mm²) at 37° C. in a humidified atmosphere of 5% CO₂. Confluency of cells should be 80-90% on day of plating. Cells are rinsed with 10 ml of PBS and cells are re-suspended by addition of culture medium and trituration with a 25 ml pipette.

The cells are seeded at a density of approximately 1×10⁶ cells/ml (100 μl/well) in black-walled, clear bottom, 96-well plates pre-treated with 10 mg/l poly-D-lysin (75 μl/well for ≧30 min). Plated cells were allowed to proliferate for 24 h before loading with dye.

Loading with Fluo-4/AM

Fluo-4/AM (1 mg, Molecular Probes) is added 912 μl DMSO. The Fluo-4/AM stock solution (1 mM) is diluted with culture medium to a final concentration of 2 μM.

The culture medium is aspirated from the wells, and 50 μl of the Fluo-4/AM loading solution is added to each well. The cells are incubated at 37° C. for 30 min.

Calcium Measurements

After the loading period, the loading solution is aspirated and the cells are washed twice with 100 μl modified FLIPR medium (145 mM NaCl, 5 mM KCl, 5 mM CaCl₂, 1 mM MgCl₂, 10 mM HEPES, pH 7.4) to remove extracellular dye. Following the second wash, 100 μl modified FLIPR medium is added to each well and the fluorescence is measured in FLIPR.

FLIPR Settings (ASIC1a)

Temperature: Room temperature (20-22° C.)

First addition: 50 μl test solution at a rate of 30 μl/sec and a starting height of 100 μl

Second addition: 50 μl MES solution (20 mM, 5 mM final concentration) at a rate of 35 μl/sec and a starting height of 150 μl.

Reading intervals: pre-incubation—10 sec×7 and 3 sec×3 antagonist phase—3 sec×17 and 10 sec×12

Addition plates (compound test plate and MES plate) are placed on the right and left positions in the FLIPR tray, respectively. Cell plates are placed in the middle position and the ASIC1a program is effectuated. FLIPR will then take the appropriate measurements in accordance with the interval settings above. Fluorescence obtained after stimulation is corrected for the mean basal fluorescence (in modified FLIPR medium).

Hit confirmation

The MES-induced calcium response, in the presence of test substance, is expressed relatively to the MES response alone. Test substances that block the MES-induced calcium response by more that 50% (peak value) are re-tested in triplicates. Confirmed hits are picked for further characterization.

Characterization of Active Substances

Full concentration/response curves are generated and IC50 values (the concentration of the test substance which inhibits 50% of the MES-induced calcium response) are calculated based on peak values.

Results of the FLIPR studies for effect on ASIC1a (HEK cells) are shown in Table 4 below:

TABLE 4

| Compound | Results (ASIC1a) |
|---|---|
| (Compound A) | Between 30 and 45% inhibition at 1 μM |
| | (IC₅₀ between 1 and 4 μM) |
| 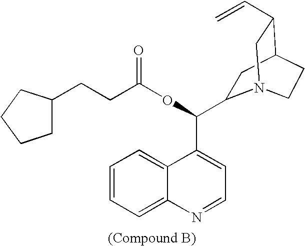 | Between 30 and 45% inhibition at 1 μM |

### Example 6

### Cloning and Expression of AISCs

The cDNA for ASIC1a and ASIC3 can be cloned from rat poly(A)⁺ mRNA and put into expression vectors according to Hesselager et al. (J Biol Chem. 279(12):11006-15 2004). All constructs are expressed in CHO-K1 cells (ATCC no. CCL61). CHO-K1 cells are cultured at 37° C. in a humidified atmosphere of 5% CO₂ and 95% air and passaged twice every week. The cells are maintained in DMEM (10 mM HEPES, 2 mM glutamax) supplemented with 10% fetal bovine serum and 2 mM L-proline (Life Technologies). CHO-K1 cells are co-transfected with plasmids containing ASICs and a plasmid encoding enhanced green fluorescent protein (EGFP) using the lipofectamine PLUS transfection kit (Life Technologies) according to the manufacturer's protocol. For each transfection it is attempted to use an amount of DNA that yield whole-cell currents within a reasonable range (0.5 nA -10 nA), in order to avoid saturation of the patch-clamp amplifier (approximately 50 ng for ASIC1a and ASIC3). Electrophysiological measurements are performed 16-48 hours after transfection. The cells are trypsinized and seeded at 3.5 mm glass coverslips, precoated with poly-D-lysine, at the same day as the electrophysiological recordings were performed.

TABLE 5

| | | | |
|---|---|---|---|
| | Modality | | Non-limiting examples of potential |
| Model Name | tested | Brief Description | clinical indications (Reference) |

ACUTE PHASIC PAIN

| | | | |
|---|---|---|---|
| Tail-flick | Thermal | Tip of tail of rats is immersed if hot water and time | Acute nociceptive pain |
| | | to withdrawal from water is measured. Alternatively, | (Hardy et al. Am J Physiol 1957; |
| | | a radiant heat source is applied to the tail and time | 189: 1-5.; Ben-Bassat et al. |
| | | to withdrawal is determined. Analgesic effect is | Arch Intern Pharmacodyn Ther 1959; |
| | | evidenced by a prolongation of the latency period | 122: 434-47.) |
| hot-plate | Thermal | Rats walk over a heated surface with increasing | Acute nociceptive pain |
| | | temperature and observed for specific nociceptive | (Woolfe et al. J Pharmacol Exp |
| | | behavior such paw licking, jumping. Time to | Ther 1944; 80: 300-7.) |
| | | appearance of such behavior is measured. | |
| | | Analgesic effects are evidenced by a prolonged | |
| | | latency. | |
| Paw | Thermal | A focused beam of light is projected onto a small | Acute nociceptive pain |
| withdrawal | | surface of the hind leg of a rat with increasing | (Yeomans et al. Pain 1994; 59: 85-94.) |
| | | temperature. Time to withdrawal is measured. | |
| | | Analgesic effect translates into a prolonged latency | |
| | Mechanical | An increasing calibrated pressure is applied to the | Acute nociceptive pain |
| | | paw of rats with a blunt pin. Pressure intensity is | (Green et al. Br J Pharmacol 1951; 6: |
| | | measured. | 572-85.; Randall et al. Arch Int Pharmacodyn |
| | | | Ther 1957; 111: 409-19) |

ACUTE TONIC PAIN

| | | | |
|---|---|---|---|
| Formalin test | Chemical | Formalin is injected into the hind paw of animals | Inflammatory pain |
| | | (rat, mice) and the pain behavior is scored (e.g. | (Dubuisson et al. Pain 1977; 4: 161-74.; |
| | | paw licking/unit of time) | Wheeler-Aceto et al. Psychopharmacology |
| | | | (Berl) 1991; 104: 35-44.) |
| Writhing Test | Chemical | Acetic acid is injected into the peritoneal cavity of a | Visceral pain, peritonitis |
| | | rat. The outcome measure is the number of | (Loux et al. Arzneimittelforschung |
| | | abdominal cramps per unit of time. A decrease in | 1978; 28: 1644-7.) |
| | | cramps is evidence of analgesic effect | |

HYPERALGESIA MODELS/CHRONIC INFLAMMATORY PAIN MODELS

| | | | |
|---|---|---|---|
| Hargreaves | Chemical and | A sensitizing agent (carrageenin, turpentine etc.) is | Chronic pain associated with tissue |
| | mechanical | injected into the paw of rats creating a local | inflammation, e.g. post-surgical pain, |
| | | inflammation and sensitivities to mechanical | (Hargreaves et al.. Pain 1988; 32: 77-88.) |
| | | stimulation are measured with comparison to the | |
| | | contralateral non-sensitized paw | |
| Yeomans | Chemical and | Rat hind paw in injected with capsaicin, a | Chronic pain associated with tissue |
| model | thermal | sensitizing agent for small C-fibers or DMSO, a | inflammation, e.g. post-surgical pain |
| | | sensitizing agent for A-delta fibers. A radiant heat is | (Yeomans et al. Pain 1994; 59: 85-94.; |
| | | applied with different gradient to differentially | Otsuki et al. Brain Res 1986; 365: 235-240.) |
| | | stimulate C-fibers or A-delta fibers and discriminate | |
| | | between the effects mediated by both pathways | |
| Freund's | Thermal, | Intracapsular injection of FCA or urate crystals | Arthritis |
| Complete | and/or | creates an inflammation of the joints. Animals are | (Coderre et al. Pain 1987; 28: |
| Adjuvant | Mechanical | tested for hyperalgesia, allodynia and behavioral | 379-393.; Butler et al. Pain 1992; 48: 73-81.) |
| (FCA) or | | pain responses. | |
| Urate crystal | | | |
| model | | | |

CHRONIC MALIGNANT PAIN (CANCER PAIN)

| | | |
|---|---|---|
| Bone Cancer | In this model, osteolytic mouse sarcoma | Bone cancer pain |
| Model | NCTC2472 cells are used to induce bone cancer by | (Schwei et al., J. Neurosci. |
| | injecting tumor cells into the marrow space of the | 1999; 19: 10886-10897.) |
| | femur bone and sealing the injection site | |
| Cancer | Meth A sarcoma cells are implanted around the | Malignant neuropathic pain |
| invasion pain | sciatic nerve in BALB/c mice and these animals | (Shimoyama et al., Pain 2002; 99: 167-174.) |
| model (CIP) | develop signs of allodynia and thermal hyperalgesia | |
| | as the tumor grows, compressing the nerve. | |
| | Spontaneous pain (paw lifting) is also visible. | |

CHRONIC NON-MALIGNANT PAIN

| | | | |
|---|---|---|---|
| Muscle Pain | Chemical, | Repeated injections of acidic saline into one | Fibromyalgia |
| | mechanical | gastrocnemius muscle produces bilateral, long- | (Sluka et al. Pain 2003; 106: 229-239.) |
| | and thermal | lasting mechanical hypersensitivity of the paw (i.e. | |
| | | hyperalgesia) without associated tissue damage | |
| UV-irradiation | | Exposure of the rat hind paw to UV irradiation | Inflammatory pain associated with first- |
| | | produces highly reliable and persistent allodynia. | and second-degree burns. |
| | | Various irradiation periods with UV-B produce skin | (Perkins et al. Pain 1993; |
| | | inflammation with different time courses | 53: 191-197.) |

CHRONIC NEUROPATHIC PAIN

| | | |
|---|---|---|
| Chronic | Loose chronic ligature of the sciatic nerve. Thermal | Clinical Neuropathic pain: nerve |
| Constriction | or mechanical sensitivities are tested using Von | compression and direct mechanical |
| Injury (CCI) or | Frey hairs or the paw withdrawal test | neuronal damage might be relevant |
| Bennett and | | clinical comparisons |
| Xie model | | (Bennett & Xie, Neuropharmacology |
| | | 1984; 23: 1415-1418.) |
| Seltzer model | Partial tight ligature of the sciatic nerve. Thermal or | Same as above |
| | mechanical sensitivities are tested using Von Frey | (Seltzer et al. Pain 1988; 33: 87-107.) |
| | hairs or the paw withdrawal test | |
| Chung's | Tight ligation of one of the two spinal nerves of the | Same as above: root compression |
| model or | sciatic nerve. Thermal or mechanical sensitivities | might be a relevant clinical comparison |
| Spinal Nerve | are tested using Von Frey hairs or the paw | (Kim and Chung, Pain 1990; 41: 235-251.) |
| Ligation | withdrawal test | |
| model (SNL) | | |
| Streptozotocin | Rats are treated with STZ which destroys the | Diabetic neuropathies |
| (STZ) | Langerhans cells of the pancreas and leading to a | (Courteix et al., Pain 1993; 53: 81-88.) |
| | rapid development of hyperglycaemia and ensuing | |
| | periphral neuropathies | |

We claim:

1. A method of modulating the activity of a gated ion channel, comprising contacting a cell expressing a gated ion channel with an effective amount of a compound represented by the Formula 1, 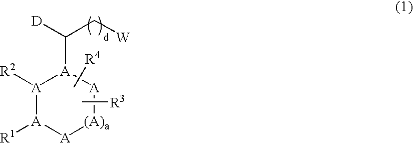
or a pharmaceutically acceptable salt thereof,
wherein
A is, independently, either an sp²- or sp³-hybridized carbon or nitrogen atom; D is selected from the group consisting of —H, —OH, halogen, —(CH₂)₀₋₆Y, —O—(CH₂)₀₋₆Y, wherein Y is selected from —H, —CN, —CO₂H, —SO₃H, —SO₂H, —PO₃H₂, —NO₂, —SSO₃H, halomethyl, dihalomethyl, trihalomethyl, N-methyl-piperidinyl, morpholinyl, hydroxyphenyl, phenyl, piperazinyl, cyclopropyl, cyclopentyl, cyclohexyl, pyridinyl, 5H-tetrazolyl, triazolyl or piperidinyl; R⁹(CH₂)₀₋₆COO—, —N(R⁹)(CH₂)₀₋₆COO(R⁹), —O(CH₂)₀₋₆(R⁹), —(CH₂)₁₋₆COO(R⁹), —(CH₂)₁₋₆N(R⁹)COO(R⁹), —(CH₂)₁₋₆N(R⁹)CO(R⁹), —(CH₂)₁₋₆CONH(R⁹), ═NOR⁹, wherein R⁹ is selected from the group consisting of —H, —C₁₋₄-alkyl, N-methyl-piperidinyl, morpholinyl, hydroxyphenyl, phenyl, piperazinyl, cyclopropyl, cyclopentyl, cyclohexyl, pyridinyl, 5H-tetrazolyl, triazolyl, or piperidinyl; —N(X¹)X², —SO₂N(X¹)X², wherein X¹ and X² are each, independently, H, aryl, or C₁-C₆-alkyl; salts thereof, esters thereof, and any combination thereof;
W is selected from the group consisting of —H, —OH, halogen, —(CH₂)₀₋₆Y, —O—(CH₂)₀₋₆Y, wherein Y is selected from —H, —CN—CO₂H, —SO₃H, —SO₂H, —PO₃H₂, —NO₂, —SSO₃H, halomethyl, dihalomethyl, trihalomethyl, N-methyl-piperidinyl, morpholinyl, hydroxyphenyl, phenyl, piperazinyl, cyclopropyl, cyclopentyl, cyclohexyl, pyridinyl, 5H-tetrazolyl, triazolyl or piperidinyl; R⁹(CH₂)₀₋₆COO—, —N(R⁹)(CH₂)₀₋₆COO(R⁹), —O(CH₂)₀₋₆(R⁹), —(CH₂)₁₋₆COO(R⁹), —(CH₂)₁₋₆N(R⁹)COO(R⁹), —(CH₂)₁₋₆N(R⁹)CO(R⁹), —(CH₂)₁₋₆CONH(R⁹), ═NOR⁹, wherein R⁹ is selected from the group consisting of —H, —C₁₋₄-alkyl, N-methyl-piperidinyl, morpholinyl, hydroxyphenyl, phenyl, piperazinyl, cyclopropyl, cyclopentyl, cyclohexyl, pyridinyl, 5H-tetrazolyl, triazolyl, or piperidinyl; —N(X¹)X², —SO₂N(X¹)X², or —(CH₂)₁₋₆SO₂N(X¹)X², wherein X¹ and X² are each, independently, H, aryl, C₁-C₆-alkyl; salts thereof, esters thereof, and any combination thereof; and any combination thereof; or the formula 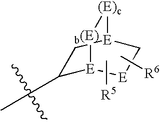
wherein E is, independently, either an sp²- or sp³-hybridized carbon or nitrogen atom; R⁵ and R⁶ are each, independently, —H, —OH, —(CH₂)₀₋₆Y, —O—(CH₂)₀₋₆Y, wherein Y is selected from —CHCH₂, —CH₂CHCH₂, —H, —OH, —CN, halo, —NO₂, morpholinyl, hydroxyphenyl, phenyl, piperazinyl, cyclopropyl, cyclopentyl, cyclohexyl, pyridinyl, 5H-tetrazolyl, triazolyl, piperidinyl, alkylcarbonyl, alkylthiocarbonyl, alkoxycarbonyl, aminocarbonyl, —CO₂H; —SO₃H; —SO₂H; —SO₂NH₂, —SSO₃H, —PO₃H₂; —NO₂, —SH, —OSO₃H, —OC(O)(OH), —O—, —S—, halomethyl, dihalomethyl, trihalomethyl, —SO₂N(X¹)X² or N(X¹)X², wherein X¹ and X² are each, independently, H, aryl, C₁-C₆-alkyl, esters thereof; salts thereof, and any combination thereof;
R¹, R², R³, R⁴, are each, independently, selected from the group consisting of —H, —OH, halogen, —(CH₂)₀₋₆Y, —O—(CH₂)₀₋₆Y, wherein Y is selected from —H, —CN, —CO₂H, —SO₃H, —SO₂H, —PO₃H₂, —NO₂, —SSO₃H, halomethyl, dihalomethyl, trihalomethyl, N-methyl-piperidinyl, morpholinyl, hydroxyphenyl, phenyl, piperazinyl, cyclopropyl, cyclopentyl, cyclohexyl, pyridinyl, 5H-tetrazolyl, triazolyl or piperidinyl; R⁹(CH₂)₀₋₆COO—, —N(R⁹)(CH₂)₀₋₆COO(R⁹), —O(CH₂)₀₋₆(R⁹), —(CH₂)₁₋₆COO(R⁹), —(CH₂)₁₋₆N(R⁹)COO(R⁹), —(CH₂)₁₋₆N(R⁹)CO(R⁹), —(CH_{2l )}₁₋₆CONH(R⁹), ═NOR⁹, wherein R⁹ is selected from the group consisting of —H, —C₁₋₄-alkyl, N-methyl-piperidinyl, morpholinyl, hydroxyphenyl, phenyl, piperazinyl, cyclopropyl, cyclopentyl, cyclohexyl, pyridinyl, 5H-tetrazolyl, triazolyl, or piperidinyl; —N(X¹)X², —SO₂N(X¹)X² wherein X¹ and X² are each, independently, H, aryl, C₁-C₆-alkyl; salts thereof, esters thereof, and any combination thereof;
R¹ and R² can also form together for a fused 5- or 6-membered ring composed of one of the following bridging bivalent radicals (reading from R¹ to R²):
—GR¹⁰-CH₂—CH₂—
—CH₂-GR¹⁰—CH₂—
—CH₂—CH₂-GR¹⁰—
-GR¹⁰—CH₂—CH₂—CH₂—
—CH₂-GR¹⁰—CH₂—CH₂—
—CH₂—CH₂-GR¹⁰—CH₂—
—CH₂—CH₂—CH₂-GR¹⁰—
-GR¹⁰═CH—CH═CH—
—CH=GR¹⁰—CH═CH—
—CH═CH-GR¹⁰═CH—
—CH═CH—CH=GR¹⁰—
wherein G is either an sp²- or sp³-hybridized carbon or nitrogen atom; wherein R¹⁰ has the meaning set forth for R⁶;
a, b, c and d are each 0 or 1.

2-4. (canceled)

5. The method of claim 1, wherein the compound is represented by the Formula 2, 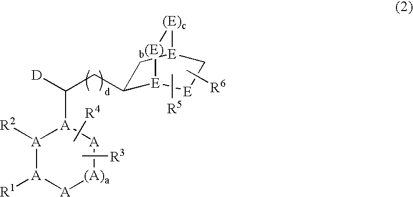
or a pharmaceutically acceptable salt thereof,
wherein
A and E are each, independently, an sp²- or sp³-hybridized carbon or nitrogen atom;
D is selected from the group consisting of —H, —OH, halogen, —(CH₂)₀₋₆Y, —O—(CH₂)₀₋₆Y, wherein Y is selected from —H, —CN, —CO₂H, —SO₃H, —SO₂H, —PO₃H₂, —NO₂, —SSO₃H, halomethyl, dihalomethyl, trihalomethyl, N-methyl-piperidinyl, morpholinyl, hydroxyphenyl, phenyl, piperazinyl, cyclopropyl, cyclopentyl, cyclohexyl, pyridinyl, 5H-tetrazolyl, triazolyl or piperidinyl; R⁹(CH₂)₀₋₆COO—, —N(R⁹)(CH₂)₀₋₆COO(R⁹), —O(CH₂)₀₋₆(R⁹), —(CH₂)₁₋₆COO(R⁹), —(CH₂)₁₋₆N(R⁹)COO(R⁹), —(CH₂)₁₋₆N(R⁹)CO(R⁹), —(CH₂)₁₋₆CONH(R⁹), ═NOR⁹, wherein R⁹ is selected from the group consisting of —H, —C₁₋₄-alkyl, N-methyl-piperidinyl, morpholinyl, hydroxyphenyl, phenyl, piperazinyl, cyclopropyl, cyclopentyl, cyclohexyl, pyridinyl, 5H-tetrazolyl, triazolyl, or piperidinyl; —N(X¹)X², —SO₂N(X¹)X², wherein X¹ and X² are each, independently, H, aryl, C₁-C₆-alkyl; salts thereof, esters thereof, and any combination thereof;
R¹, R², R³, R⁴, R⁵ and R⁶, are each, independently, —H, —OH, halo, C₁-C₆-alkyl, —O—C₁₋₆-alkyl, —CHCH₂, —CH₂CHCH₂, —(CH₂)₀₋₆Y, —O—(CH₂)₀₋₆Y, wherein Y is selected from —H, —CN, CO₂H, —SO₃H, —SO₂H, —PO₃H₂, —NO₂, —SSO₃H, halomethyl, dihalomethyl, trihalomethyl, N-methyl-piperidinyl, morpholinyl, hydroxyphenyl, phenyl, piperazinyl, cyclopropyl, cyclopentyl, cyclohexyl, pyridinyl, 5H-tetrazolyl, triazolyl or piperidinyl; R⁹(CH₂)₀₋₆COO—, —N(R⁹)(CH₂)₀₋₆COO(R⁹), —O(CH₂)₀₋₆(R⁹), —(CH₂)₁₋₆COO(R⁹), —(CH₂)₁₋₆N(R⁹)COO(R⁹), —(CH₂)₁₋₆N(R⁹)CO(R⁹), —(CH₂)₁₋₆CONH(R⁹), ═NOR⁹, wherein R⁹ is selected from the group consisting of —H, —C₁₋₄-alkyl, N-methyl-piperidinyl, morpholinyl, hydroxyphenyl, phenyl, piperazinyl, cyclopropyl, cyclopentyl, cyclohexyl, pyridinyl, 5H-tetrazolyl, triazolyl, or piperidinyl; —N(X¹)X², —SO₂N(X¹)X² wherein X¹ and X² are each, independently, H, aryl, C₁-C₆-alkyl; salts thereof, esters thereof, and any combination thereof,
R¹ and R² can also form together for a fused 5- or 6-membered ring composed of one of the following bridging bivalent radicals (reading from R¹ to R²):
-GR¹⁰—CH₂—CH₂—
—CH₂-GR¹⁰—CH₂—
—CH₂—CH₂-GR¹⁰—
-GR¹⁰—CH₂—CH₂—CH₂—
—CH₂-GR¹⁰—CH₂—CH₂—
—CH₂—CH₂-GR¹⁰—CH₂—
—CH₂—CH₂—CH₂-GR¹⁰—
-GR¹⁰═CH—CH═CH—
—CH=GR¹⁰—CH═CH—
—CH═CH-GR¹⁰═CH—
—CH═CH—CH=GR¹⁰—
wherein G is either an sp²- or sp³-hybridized carbon or nitrogen atom; wherein R¹⁰ has the meaning set forth for R⁶;
a, b, c and d are each 0 or 1.

6-9. (canceled)

10. The method of claim 1, wherein the compound is represented by the Formula 3, 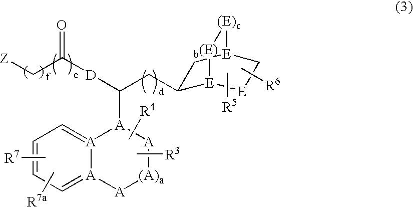
or a pharmaceutically acceptable salt thereof,
wherein
A and E are each, independently, an sp²- or sp³-hybridized carbon or nitrogen atom;
D is selected from the group consisting of —CH₂CHCH—, —CH₂CHCHCH₂—, —O—, —[(CH₂)₁₋₆]—, —O—(CH₂)₁₋₆—, —O—(CH₂)₁₋₆—N(R⁹)—, —(CH₂)₁₋₆—N(R⁹)—, N(R⁹)—, wherein R⁹ is selected from the group consisting of —H, —C₁₋₄-alkyl, N-methyl-piperidinyl, morpholinyl, hydroxyphenyl, phenyl, piperazinyl, cyclopropyl, cyclopentyl, cyclohexyl, pyridinyl, 5H-tetrazolyl, triazolyl or piperidinyl;
R³, R⁴, R⁵, R⁶, R⁷ and R^{7a} are each, independently, —H, —OH, —(CH₂)₀₋₆Y, —O—(CH₂)₀₋₆Y, wherein Y is selected from —H, —OH, —CN, halo, —CHCH₂, —CH₂CHCH₂, —NO₂, morpholinyl, hydroxyphenyl, phenyl, piperazinyl, cyclopropyl, cyclopentyl, cyclohexyl, pyridinyl, 5H-tetrazolyl, triazolyl, piperidinyl, alkylcarbonyl, alkylthiocarbonyl, alkoxycarbonyl, aminocarbonyl, —CO₂H; —SO₃H; —SO₂H; —SO₂NH₂, —SSO₃H, —PO₃H₂; —NO₂, —SH, —OSO₃H, —OC(O)(OH), —O—, —S—, halomethyl, dihalomethyl, trihalomethyl, —SO₂N(X¹)X² or N(X¹)X², wherein X¹ and X² are each, independently, H, aryl, C₁-C₆-alkyl, esters thereof; salts thereof, and any combination thereof;
Z is selected from the group consisting of —H, —OH, —(CH₂)₀₋₆Y, —O—(CH₂)₀₋₆Y, wherein Y is selected from —H, —OH, —CN, halo, —CHCH₂, —CH₂CHCH₂, —NO₂, morpholinyl, hydroxyphenyl, phenyl, piperazinyl, cyclopropyl, cyclopentyl, cyclohexyl, pyridinyl, 5H-tetrazolyl, triazolyl, piperidinyl, alkylcarbonyl, alkylthiocarbonyl, alkoxycarbonyl, aminocarbonyl, —CO₂H, —SO₃H; —SO₂H; —SO₂NH₂, —SSO₃H, —PO₃H₂; —NO₂, —SH, —OSO₃H, —OC(O)(OH), —O—, —S—, halomethyl, dihalomethyl, trihalomethyl, —SO₂N(X¹)X² or N(X¹)X², wherein X¹ and X² are each, independently, H, aryl, C₁-C₆-alkyl, esters thereof; salts thereof, and any combination thereof,
R⁷ and R^{7a} can also form together for a fused 5- or 6-membered ring composed of one of the following bridging bivalent radicals:
—O—CH₂—CH₂—
—CH₂—O—CH₂—
—CH₂—CH₂—O—
—CH₂—CH₂—CH₂—
—O—CH₂—O—
—CH₂—CH₂—CH₂—CH₂—
a, b, c, d and e are each 0 or 1
f is 0, 1, 2, 3, 4, 5 or 6.

11-20. (canceled)

21. The method of claim 1, wherein the compound is represented by the Formula 4, 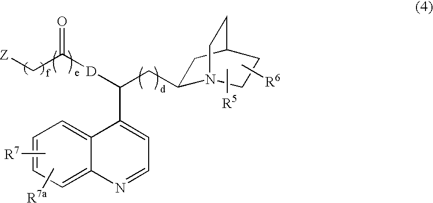
or a pharmaceutically acceptable salt thereof,
wherein
D is selected from the group consisting of —O—, —(CH₂)₁₋₆—, —N(R⁹)—, wherein R⁹ is selected from the group consisting of —H, —C₁₋₄-alkyl, N-methyl-piperidinyl, morpholinyl, hydroxyphenyl, phenyl, piperazinyl, cyclopropyl, cyclopentyl, cyclohexyl, pyridinyl, 5H-tetrazolyl, triazolyl, or piperidinyl;
R⁵, R⁶, R⁷ and R^{7a} are each, independently, —H, —OH, —(CH₂)₀₋₆Y, —O—(CH₂)₀₋₆Y, wherein Y is selected from —H, —OH, —CN, halo, —CHCH₂, —CH₂CHCH₂, —NO₂, morpholinyl, hydroxyphenyl, phenyl, piperazinyl, cyclopropyl, cyclopentyl, cyclohexyl, pyridinyl, 5H-tetrazolyl, triazolyl, piperidinyl, alkylcarbonyl, alkylthiocarbonyl, alkoxycarbonyl, aminocarbonyl, —CO₂H; —SO₃H; —SO₂H; —SO₂NH₂, —SSO₃H, —PO₃H₂; —NO₂, —SH, —OSO₃H, —OC(O)(OH), —O—, —S—, halomethyl, dihalomethyl, trihalomethyl, —SO₂N(X¹)X² or N(X¹)X², wherein X¹ and X² are each, independently, H, aryl, C₁-C₆-alkyl, esters thereof, salts thereof, and any combination thereof;
Z is selected from the group consisting of —H, —OH, —(CH₂)₀₋₆Y, —O—(CH₂)₀₋₆Y, wherein Y is selected from —H, —OH, —CN, halo, —CHCH₂, —CH₂CHCH₂, —NO₂, morpholinyl, hydroxyphenyl, phenyl, piperazinyl, cyclopropyl, cyclopentyl, cyclohexyl, pyridinyl, 5H-tetrazolyl, triazolyl, piperidinyl, alkylcarbonyl, alkylthiocarbonyl, alkoxycarbonyl, aminocarbonyl, —CO₂H, —SO₃H; —SO₂H; —SO₂NH₂, —SSO₃H, —PO₃H₂; —NO₂, —SH, —OSO₃H, —OC(O)(OH), —O—, —S—, halomethyl, dihalomethyl, trihalomethyl, —SO₂N(X¹)X² or N(X¹)X², wherein X¹ and X² are each, independently, H, aryl, C₁-C₆-alkyl, esters thereof, salts thereof, and any combination thereof;
R⁷ and R^{7a} can also form together for a fused 5- or 6-membered ring composed of one of the following bridging bivalent radicals:
—O—CH₂—CH₂—
—CH₂—O—CH₂—
—CH₂—CH₂—O—
—CH₂—CH₂—CH₂—
—O—CH₂—O—
—CH₂—CH₂—CH₂—CH₂—
d and e are each, independently, 0 or 1;
f is 0, 1, 2, 3, 4, 5 or 6.

22-31. (canceled)

32. The method of claim 1, wherein the compound is represented by the Formula 5, 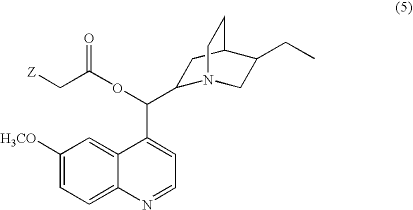
or a pharmaceutically acceptable salt thereof,
wherein
Z is selected from the group consisting of —H, —OH, —(CH₂)₀₋₆Y, —O—(CH₂)₀₋₆Y, wherein Y is selected from —H, C₁-C₆-alkyl, —OH, —CN, halo, —CHCH₂, —CH₂CHCH₂, —NO₂, morpholinyl, hydroxyphenyl, phenyl, piperazinyl, cyclopropyl, cyclopentyl, cyclohexyl, pyridinyl, 5H-tetrazolyl, triazolyl, piperidinyl, alkylcarbonyl, alkylthiocarbonyl, alkoxycarbonyl, aminocarbonyl, —CO₂H, —SO₃H; —SO₂H; —SO₂NH₂, —SSO₃H, —PO₃H₂; —NO₂, —SH, —OSO₃H, —OC(O)(OH), —O—, —S—, halomethyl, dihalomethyl, trihalomethyl, —SO₂N(X¹)X² or N(X¹)X², wherein X¹ and X² are each, independently, H, aryl, C₁-C₆-alkyl, esters thereof; salts thereof, and any combination thereof.

33-34. (canceled)

35. The method of claim 1, wherein contacting the cells with an effective amount of the compound inhibits the activity of the gated ion channel.

36. The method of claim 1, wherein the gated ion channel is comprised of at least one subunit selected from the group consisting of a member of the DEG/ENaC, P2X, and TRPV gene superfamilies.

37. The method of claim 1, wherein the gated ion channel is comprised of at least one subunit selected from the group consisting of αENaC, βENaC, γENaC, δENaC, ASIC1a, ASIC1b, ASIC2a, ASIC2b, ASIC3, ASIC4, BLINaC, hINaC, P2X₁, P2X₂, P2X₃, P2X₄, P2X₅, P2X₆, P2X₇, TRPV1, TRPV2, TRPV3, TRPV4, TRPV5, and TRPV6.

38. The method of claim 37, wherein the gated ion channel is homomultimeric.

39. The method of claim 37, wherein the gated ion channel is heteromultimeric.

40. The method of claim 36, wherein the DEG/ENaC gated ion channel is comprised of at least one subunit selected from the group consisting of αENaC, βENaC, γENaC, δENaC, BLINaC, hINaC, ASIC1a, ASIC1b, ASIC2a, ASIC2b, ASIC3, and ASIC4.

41. The method of claim 36, wherein the DEG/ENaC gated ion channel is comprised of at least one subunit selected from the group consisting of ASIC1a, ASIC1b, ASIC2a, ASIC2b, ASIC3, and ASIC4.

42. The method of claim 36, wherein the gated ion channel comprises ASIC1a.

43. The method of claim 36, wherein the P2X gated ion channel comprises at least one subunit selected from the group consisting of P2X₁, P2X₂, P2X₃, P2X₄, P2X₅, P2X₆, and P2X₇.

44. The method of claim 36, wherein the TRPV gated ion channel comprises at least one subunit selected from the group TRPV1, TRPV2, TRPV3, TRPV4, TRPV5, and TRPV6.

45. The method of claim 39, wherein the heteromultimeric gated ion channels include the following combinations of gated ion channels: αENaC, βENaC and γENaC; αENaC, βENaC and δENaC; ASIC1a and ASIC3; ASIC1b and ASIC3; ASIC2a and ASIC3; ASIC2b and ASIC3; ASIC1a, ASIC2a and ASIC3; P2X1 and P2X2; P2X1 and P2X5; P2X2 and P2X3; P2X2 and P2X6; P2X4 and P2X6; TRPV1 and TRPV2; TRPV5 and TRPV6; and TRPV1 and TRPV4.

46. The method of claim 39, wherein the heteromultimeric gated ion channels include the following combinations of gated ion channels: ASIC1a and ASIC2a; ASIC2a and ASIC2b; ASIC1a and ASIC3; ASIC1b and ASIC3; and ASIC3 and ASIC2b.

47. The method of claim 1, wherein the activity of the gated ion channel is associated with pain.

48. The method of claim 1, wherein the activity of the gated ion channel is associated with an inflammatory disorder.

49. The method of claim 1, wherein the activity of the gated ion channel is associated with a neurological disorder.

50-69. (canceled)

70. A method of treating pain in a subject in need thereof, comprising administering to the subject an effective amount of a compound of Formula 1, 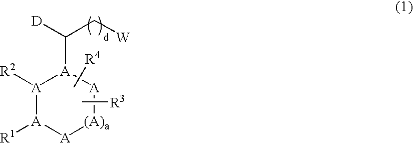
or a pharmaceutically acceptable salt thereof,
wherein
A is, independently, either an sp²- or sp³-hybridized carbon or nitrogen atom; D is selected from the group consisting of —H, —OH, halogen, —(CH₂)₀₋₆Y, —O—(CH₂)₀₋₆Y, wherein Y is selected from —H, —CN, —CO₂H, —SO₃H, —SO₂H, —PO₃H₂, —NO₂, —SSO₃H, halomethyl, dihalomethyl, trihalomethyl, N-methyl-piperidinyl, morpholinyl, hydroxyphenyl, phenyl, piperazinyl, cyclopropyl, cyclopentyl, cyclohexyl, pyridinyl, 5H-tetrazolyl, triazolyl or piperidinyl; R⁹(CH₂)₀₋₆COO—, —N(R⁹)(CH₂)₀₋₆COO(R⁹), —O(CH₂)₀₋₆(R⁹), —(CH₂)₁₋₆COO(R⁹), —(CH₂)₁₋₆N(R⁹)COO(R⁹), —(CH₂)₁₋₆N(R⁹)CO(R⁹), —(CH₂)₁₋₆CONH(R⁹), ═NOR⁹, wherein R⁹ is selected from the group consisting of —H, —C₁₋₄-alkyl, N-methyl-piperidinyl, morpholinyl, hydroxyphenyl, phenyl, piperazinyl, cyclopropyl, cyclopentyl, cyclohexyl, pyridinyl, 5H-tetrazolyl, triazolyl, or piperidinyl; —N(X¹)X², —SO₂N(X¹)X², wherein X¹ and X² are each, independently, H, aryl, or C₁-C₆-alkyl; salts thereof, esters thereof, and any combination thereof;
W is selected from the group consisting of —H, —OH, halogen, —(CH₂)₀₋₆Y, —O—(CH₂)₀₋₆Y, wherein Y is selected from —H, —CN—CO₂H, —SO₃H, —SO₂H, —PO₃H₂, —NO₂, —SSO₃H, halomethyl, dihalomethyl, trihalomethyl, N-methyl-piperidinyl, morpholinyl, hydroxyphenyl, phenyl, piperazinyl, cyclopropyl, cyclopentyl, cyclohexyl, pyridinyl, 5H-tetrazolyl, triazolyl or piperidinyl; R⁹(CH₂)₀₋₆COO—, —N(R⁹)(CH₂)₀₋₆COO(R⁹), —O(CH₂)₀₋₆(R⁹), —(CH₂)₁₋₆COO(R⁹), —(CH₂)₁₋₆N(R⁹)COO(R⁹), —(CH₂)₁₋₆N(R⁹)CO(R⁹), —(CH₂)₁₋₆CONH(R⁹), ═NOR⁹, wherein R⁹ is selected from the group consisting of —H, —C₁₋₄-alkyl, N-methyl-piperidinyl, morpholinyl, hydroxyphenyl, phenyl, piperazinyl, cyclopropyl, cyclopentyl, cyclohexyl, pyridinyl, 5H-tetrazolyl, triazolyl, or piperidinyl; —N(X¹)X², —SO₂N(X¹)X², or —(CH₂)₁₋₆SO₂N(X¹)X², wherein X¹ and X² are each, independently, H, aryl, C₁-C₆-alkyl; salts thereof, esters thereof, and any combination thereof; and any combination thereof; or the formula 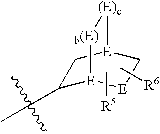
wherein E is, independently, either an sp²- or sp³-hybridized carbon or nitrogen atom; R⁵ and R⁶ are each, independently, —H, —OH, —(CH₂)₀₋₆Y, —O—(CH₂)₀₋₆Y, wherein Y is selected from —H, —OH, —CN, halo, —CHCH₂, —CH₂CHCH₂, —NO₂, morpholinyl, hydroxyphenyl, phenyl, piperazinyl, cyclopropyl, cyclopentyl, cyclohexyl, pyridinyl, 5H-tetrazolyl, triazolyl, piperidinyl, alkylcarbonyl, alkylthiocarbonyl, alkoxycarbonyl, aminocarbonyl, —CO₂H; —SO₃H; —SO₂H; —SO₂NH₂, —SSO₃H, —PO₃H₂; —NO₂, —SH, —OSO₃H, —OC(O)(OH), —O—, —S—, halomethyl, dihalomethyl, trihalomethyl, —SO₂N(X¹)X² or N(X¹)X², wherein X¹ and X² are each, independently, H, aryl, C₁-C₆-alkyl, esters thereof; salts thereof, and any combination thereof;
R¹, R², R³, R⁴, are each, independently, selected from the group consisting of —H, —OH, halogen, —(CH₂)₀₋₆Y, —O—(CH₂)₀₋₆Y, wherein Y is selected from —H, —CN, —CO₂H, —SO₃H, —SO₂H, —PO₃H₂, —NO₂, —SSO₃H, halomethyl, dihalomethyl, trihalomethyl, N-methyl-piperidinyl, morpholinyl, hydroxyphenyl, phenyl, piperazinyl, cyclopropyl, cyclopentyl, cyclohexyl, pyridinyl, 5H-tetrazolyl, triazolyl or piperidinyl; R⁹(CH₂)₀₋₆COO—, —N(R⁹)(CH₂)₀₋₆COO(R⁹), —O(CH₂)₀₋₆(R⁹), —(CH₂)₁₋₆COO(R⁹), —(CH₂)₁₋₆N(R⁹)COO(R⁹), —(CH₂)₁₋₆N(R⁹)CO(R⁹), —(CH₂)₁₋₆CONH(R⁹), ═NOR⁹, wherein R⁹ is selected from the group consisting of —H, —C₁₋₄-alkyl, N-methyl-piperidinyl, morpholinyl, hydroxyphenyl, phenyl, piperazinyl, cyclopropyl, cyclopentyl, cyclohexyl, pyridinyl, 5H-tetrazolyl, triazolyl, or piperidinyl; —N(X¹)X², —SO₂N(X¹)X² wherein X¹ and X² are each, independently, H, aryl, C₁-C₆-alkyl; salts thereof, esters thereof, and any combination thereof;
R¹ and R² can also form together for a fused 5- or 6-membered ring composed of one of the following bridging bivalent radicals (reading from R¹ to R²):
-GR¹⁰—CH₂—CH₂—
—CH₂-GR¹⁰—CH₂—
—CH₂—CH₂-GR¹⁰—
-GR¹⁰—CH₂—CH₂—CH₂—
—CH₂-GR¹⁰—CH₂—CH₂—
—CH₂—CH₂-GR¹⁰—CH₂—
—CH₂—CH₂—CH₂-GR¹⁰—
-GR¹⁰═CH—CH═CH—
—CH=GR¹⁰—CH═CH—
—CH═CH-GR¹⁰═CH—
—CH═CH—CH=GR¹⁰—
wherein G is either an sp²- or sp³-hybridized carbon or nitrogen atom; wherein R¹⁰ has the meaning set forth for R⁶;
a, b, c and d are each 0 or 1.

71-73. (canceled)

74. The method of claim 70, wherein the compound is of the Formula 2, 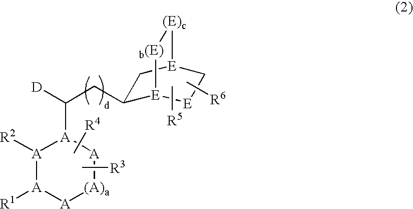
or a pharmaceutically acceptable salt thereof,
wherein
A and E are each, independently, an sp²- or sp³-hybridized carbon or nitrogen atom;
D is selected from the group consisting of —H, —OH, halogen, —(CH₂)₀₋₆Y, —O—(CH₂)₀₋₆Y, wherein Y is selected from —H, —CN, —CO₂H, —SO₃H, —SO₂H, —PO₃H₂, —NO₂, —SSO₃H, halomethyl, dihalomethyl, trihalomethyl, N-methyl-piperidinyl, morpholinyl, hydroxyphenyl, phenyl, piperazinyl, cyclopropyl, cyclopentyl, cyclohexyl, pyridinyl, 5H-tetrazolyl, triazolyl or piperidinyl; R⁹(CH₂)₀₋₆COO—, —N(R⁹)(CH₂)₀₋₆COO(R⁹), —O(CH₂)₀₋₆(R⁹), —(CH₂)₁₋₆COO(R⁹), —(CH₂)₁₋₆N(R⁹)COO(R⁹), —(CH₂)₁₋₆N(R⁹)CO(R⁹), —(CH₂)₁₋₆CONH(R⁹), ═NOR⁹, wherein R⁹ is selected from the group consisting of —H, —C₁₋₄-alkyl, N-methyl-piperidinyl, morpholinyl, hydroxyphenyl, phenyl, piperazinyl, cyclopropyl, cyclopentyl, cyclohexyl, pyridinyl, 5H-tetrazolyl, triazolyl, or piperidinyl; —N(X¹)X², —SO₂N(X¹)X², wherein X¹ and X² are each, independently, H, aryl, C₁-C₆-alkyl; salts thereof, esters thereof, and any combination thereof,
R¹, R², R³, R⁴, R⁵ and R⁶, are each, independently, —H, —OH, halo, C₁-C₆-alkyl, —O—C₁₋₆-alkyl, —CHCH₂, —CH₂CHCH₂, —(CH₂)₀₋₆Y, —O—(CH₂)₀₋₆Y, wherein Y is selected from —H, —CN, —CO₂H, —SO₃H, —SO₂H, —PO₃H₂, —NO₂, —SSO₃H, halomethyl, dihalomethyl, trihalomethyl, N-methyl-piperidinyl, morpholinyl, hydroxyphenyl, phenyl, piperazinyl, cyclopropyl, cyclopentyl, cyclohexyl, pyridinyl, 5H-tetrazolyl, triazolyl or piperidinyl; R⁹(CH₂)₀₋₆COO—, —N(R⁹)(CH₂)₀₋₆COO(R⁹), —O(CH₂)₀₋₆(R⁹), —(CH₂)₁₋₆COO(R⁹), —(CH₂)₁₋₆N(R⁹)COO(R⁹), —(CH₂)₁₋₆N(R⁹)CO(R⁹), —(CH₂)₁₋₆CONH(R⁹), —NOR⁹, wherein R⁹ is selected from the group consisting of —H, —C₁₋₄-alkyl, N-methyl-piperidinyl, morpholinyl, hydroxyphenyl, phenyl, piperazinyl, cyclopropyl, cyclopentyl, cyclohexyl, pyridinyl, 5H-tetrazolyl, triazolyl, or piperidinyl; —N(X¹)X², —SO₂N(X¹)X² wherein X¹ and X² are each, independently, H, aryl, C₁-C₆-alkyl; salts thereof, esters thereof, and any combination thereof;
R¹ and R² can also form together for a fused 5- or 6-membered ring composed of one of the following bridging bivalent radicals (reading from R¹ to R²):
-GR¹⁰—CH₂—CH₂—
—CH₂-GR¹⁰—CH₂—
—CH₂—CH₂-GR¹⁰—
-GR¹⁰—CH₂—CH₂—CH₂—
—CH₂-GR¹⁰—CH₂—CH₂—
—CH₂—CH₂-GR¹⁰—CH₂—
—CH₂—CH₂—CH₂-GR¹⁰—
-GR¹⁰═CH—CH═CH—
—CH=GR¹⁰—CH═CH—
—CH═CH-GR¹⁰═CH—
—CH═CH—CH=GR¹⁰—
wherein G is either an sp²- or sp³-hybridized carbon or nitrogen atom; wherein R¹⁰ has the meaning set forth for R⁶;
a, b, c and d are each 0 or 1.

75-78. (canceled)

79. The method of claim 70, wherein the compound is of the Formula 3, 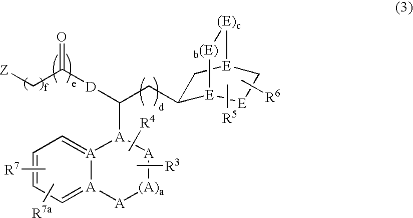
or a pharmaceutically acceptable salt thereof,
wherein
A and E are each, independently, an sp²- or sp³-hybridized carbon or nitrogen atom;
D is selected from the group consisting of —CH₂CHCH—, —CH₂CHCHCH₂—, —O—, —[(CH₂)₁₋₆]—, —O—(CH₂)₁₋₆—, —O—(CH₂)₁₋₆—N(R⁹)—, —(CH₂)₁₋₆—N(R⁹)—, N(R⁹)—, wherein R⁹ is selected from the group consisting of —H, —C₁₋₄-alkyl, N-methyl-piperidinyl, morpholinyl, hydroxyphenyl, phenyl, piperazinyl, cyclopropyl, cyclopentyl, cyclohexyl, pyridinyl, 5H-tetrazolyl, triazolyl or piperidinyl;
R³, R⁴, R⁵, R⁶, R⁷ and R^{7a} are each, independently, —H, —OH, —(CH₂)₀₋₆Y, —O—(CH₂)₀₋₆Y, wherein Y is selected from —H, —OH, —CN, halo, —CHCH₂, —CH₂CHCH₂, —NO₂, morpholinyl, hydroxyphenyl, phenyl, piperazinyl, cyclopropyl, cyclopentyl, cyclohexyl, pyridinyl, 5H-tetrazolyl, triazolyl, piperidinyl, alkylcarbonyl, alkylthiocarbonyl, alkoxycarbonyl, aminocarbonyl, —CO₂H; —SO₃H; —SO₂H; —SO₂NH₂, —SSO₃H, —PO₃H₂; —NO₂, —SH, —OSO₃H, —OC(O)(OH), —O—, —S—, halomethyl, dihalomethyl, trihalomethyl, —SO₂N(X¹)X² or N(X¹)X², wherein X¹ and X² are each, independently, H, aryl, C₁-C₆-alkyl, esters thereof; salts thereof, and any combination thereof;
Z is selected from the group consisting of —H, —OH, —(CH₂)₀₋₆Y, —O—(CH₂)₀₋₆Y, wherein Y is selected from —H, —OH, —CN, halo, —CHCH₂, —CH₂CHCH₂, —NO₂, morpholinyl, hydroxyphenyl, phenyl, piperazinyl, cyclopropyl, cyclopentyl, cyclohexyl, pyridinyl, 5H-tetrazolyl, triazolyl, piperidinyl, alkylcarbonyl, alkylthiocarbonyl, alkoxycarbonyl, aminocarbonyl, —CO₂H, —SO₃H; —SO₂H; —SO₂NH₂, —SSO₃H, —PO₃H₂; —NO₂, —SH, —OSO₃H, —OC(O)(OH), —O—, —S—, halomethyl, dihalomethyl, trihalomethyl, —SO₂N(X¹)X² or N(X¹)X², wherein X¹ and X² are each, independently, H, aryl, C₁-C₆-alkyl, esters thereof; salts thereof, and any combination thereof;
R⁷ and R^{7a} can also form together for a fused 5- or 6-membered ring composed of one of the following bridging bivalent radicals:
—O—CH₂—CH₂—
—CH₂—O—CH₂—
—CH₂—CH₂—O—
—CH₂—CH₂—CH₂—
—O—CH₂—O—
—CH₂—CH₂—CH₂—CH₂—
a, b, c, d and e are each 0 or 1
f is 0, 1, 2, 3, 4, 5 or 6.

80-89. (canceled)

90. The method of claim 70, wherein the compound is of the Formula 4, 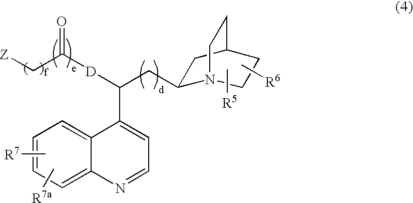
or a pharmaceutically acceptable salt thereof,
wherein
D is selected from the group consisting of —O—, —(CH₂)₁₋₆—, —N(R⁹)—, wherein R⁹ is selected from the group consisting of —H, —C₁₋₄-alkyl, N-methyl-piperidinyl, morpholinyl, hydroxyphenyl, phenyl, piperazinyl, cyclopropyl, cyclopentyl, cyclohexyl, pyridinyl, 5H-tetrazolyl, triazolyl, or piperidinyl;
R⁵, R⁶, R⁷ and R^{7a} are each, independently, —H, —OH, —(CH₂)₀₋₆Y, —O—(CH₂)₀₋₆Y, wherein Y is selected from —H, —OH, —CN, halo, —CHCH₂, —CH₂CHCH₂, —NO₂, morpholinyl, hydroxyphenyl, phenyl, piperazinyl, cyclopropyl, cyclopentyl, cyclohexyl, pyridinyl, 5H-tetrazolyl, triazolyl, piperidinyl, alkylcarbonyl, alkylthiocarbonyl, alkoxycarbonyl, aminocarbonyl, —CO₂H; —SO₃H; —SO₂H; —SO₂NH₂, —SSO₃H, —PO₃H₂; —NO₂, —SH, —OSO₃H, —OC(O)(OH), —O—, —S—, halomethyl, dihalomethyl, trihalomethyl, —SO₂N(X¹)X² or N(X¹)X², wherein X¹ and X² are each, independently, H, aryl, C₁-C₆-alkyl, esters thereof; salts thereof, and any combination thereof;
Z is selected from the group consisting of —H, —OH, —(CH₂)₀₋₆Y, —O—(CH₂)₀₋₆Y, wherein Y is selected from —H, —OH, —CN, halo, —CHCH₂, —CH₂CHCH₂, —NO₂, morpholinyl, hydroxyphenyl, phenyl, piperazinyl, cyclopropyl, cyclopentyl, cyclohexyl, pyridinyl, 5H-tetrazolyl, triazolyl, piperidinyl, alkylcarbonyl, alkylthiocarbonyl, alkoxycarbonyl, aminocarbonyl, —CO₂H, —SO₃H; —SO₂H; —SO₂NH₂, —SSO₃H, —PO₃H₂; —NO₂, —SH, —OSO₃H, —OC(O)(OH), —O—, —S—, halomethyl, dihalomethyl, trihalomethyl, —SO₂N(X¹)X² or N(X¹)X², wherein X¹ and X² are each, independently, H, aryl, C₁-C₆-alkyl, esters thereof; salts thereof, and any combination thereof;
R⁷ and R^{7a} can also form together for a fused 5- or 6-membered ring composed of one of the following bridging bivalent radicals:
—O—CH₂—CH₂—
—CH₂—O—CH₂—
—CH₂—CH₂—O—
—CH₂—CH₂—CH₂—
—O—CH₂—O—
—CH₂—CH₂—CH₂—CH₂—
d and e are each, independently, 0 or 1;
f is 0, 1, 2, 3, 4, 5 or 6.

91-100. (canceled)

101. The method of claim 70, wherein the compound is of the Formula 5, 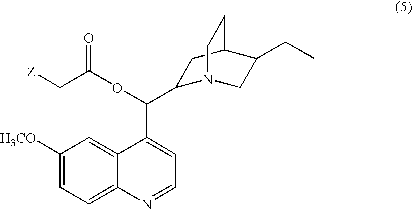
or a pharmaceutically acceptable salt thereof,
wherein
Z is selected from the group consisting of —H, —OH, —(CH₂)₀₋₆Y, —O—(CH₂)₀₋₆Y, wherein Y is selected from —H, C₁-C₆-alkyl, —OH, —CN, halo, —CHCH₂, —CH₂CHCH₂, —NO₂, morpholinyl, hydroxyphenyl, phenyl, piperazinyl, cyclopropyl, cyclopentyl, cyclohexyl, pyridinyl, 5H-tetrazolyl, triazolyl, piperidinyl, alkylcarbonyl, alkylthiocarbonyl, alkoxycarbonyl, aminocarbonyl, —CO₂H, —SO₃H; —SO₂H; —SO₂NH₂, —SSO₃H, —PO₃H₂; —NO₂, —SH, —OSO₃H, —OC(O)(OH), —O—, —S—, halomethyl, dihalomethyl, trihalomethyl, —SO₂N(X¹)X² or N(X¹)X², wherein X¹ and X² are each, independently, H, aryl, C₁-C₆-alkyl, esters thereof, salts thereof, and any combination thereof.

102-107. (canceled)

108. A method of treating an inflammatory disorder in a subject in need thereof, comprising administering to the subject an effective amount of a compound of Formula 1, 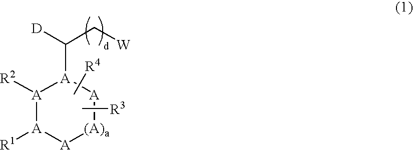
or a pharmaceutically acceptable salt thereof,
wherein
A is, independently, either an sp²- or sp³-hybridized carbon or nitrogen atom; D is selected from the group consisting of —H, —OH, halogen, —(CH₂)₀₋₆Y, —O—(CH₂)₀₋₆Y, wherein Y is selected from —H, —CN, —CO₂H, —SO₃H, —SO₂H, —PO₃H₂, —NO₂, —SSO₃H, halomethyl, dihalomethyl, trihalomethyl, N-methyl-piperidinyl, morpholinyl, hydroxyphenyl, phenyl, piperazinyl, cyclopropyl, cyclopentyl, cyclohexyl, pyridinyl, 5H-tetrazolyl, triazolyl or piperidinyl; R⁹(CH₂)₀₋₆COO—, —N(R⁹)(CH₂)₀₋₆COO(R⁹), —O(CH₂)₀₋₆(R⁹), —(CH₂)₁₋₆COO(R⁹), —(CH₂)₁₋₆N(R⁹)COO(R⁹), —(CH₂)₁₋₆N(R⁹)CO(R⁹), —(CH₂)₁₋₆CONH(R⁹), ═NOR⁹, wherein R⁹ is selected from the group consisting of —H, —C₁₋₄-alkyl, N-methyl-piperidinyl, morpholinyl, hydroxyphenyl, phenyl, piperazinyl, cyclopropyl, cyclopentyl, cyclohexyl, pyridinyl, 5H-tetrazolyl, triazolyl, or piperidinyl; —N(X¹)X², —SO₂N(X¹)X², wherein X¹ and X² are each, independently, H, aryl, or C₁-C₆-alkyl; salts thereof, esters thereof, and any combination thereof;
W is selected from the group consisting of —H, —OH, halogen, —(CH₂)₀₋₆Y, —O—(CH₂)₀₋₆Y, wherein Y is selected from —H, —CN—CO₂H, —SO₃H, —SO₂H, —PO₃H₂, —NO₂, —SSO₃H, halomethyl, dihalomethyl, trihalomethyl, N-methyl-piperidinyl, morpholinyl, hydroxyphenyl, phenyl, piperazinyl, cyclopropyl, cyclopentyl, cyclohexyl, pyridinyl, 5H-tetrazolyl, triazolyl or piperidinyl; R⁹(CH₂)₀₋₆COO—, —N(R⁹)(CH₂)₀₋₆COO(R⁹), —O(CH₂)₀₋₆(R⁹), —(CH₂)₁₋₆COO(R⁹), —selected from the group consisting of —H, —C₁₋₄-alkyl, N-methyl-piperidinyl, morpholinyl, hydroxyphenyl, phenyl, piperazinyl, cyclopropyl, cyclopentyl, cyclohexyl, pyridinyl, 5H-tetrazolyl, triazolyl, or piperidinyl; —N(X¹)X², —SO₂N(X¹)X², or —(CH₂)₁₋₆SO₂N(X¹)X², wherein X¹ and X² are each, independently, H, aryl, C₁-C₆-alkyl; salts thereof, esters thereof, and any combination thereof; and any combination thereof; or the formula 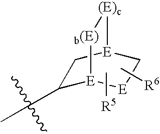
wherein E is, independently, either an sp²- or sp³-hybridized carbon or nitrogen atom; R⁵ and R⁶ are each, independently, —H, —OH, —(CH₂)₀₋₆Y, —O—(CH₂)₀₋₆Y, wherein Y is selected from —H, —OH, —CN, halo, —CHCH₂, —CH₂CHCH₂, —NO₂, morpholinyl, hydroxyphenyl, phenyl, piperazinyl, cyclopropyl, cyclopentyl, cyclohexyl, pyridinyl, 5H-tetrazolyl, triazolyl, piperidinyl, alkylcarbonyl, alkylthiocarbonyl, alkoxycarbonyl, aminocarbonyl, —CO₂H; —SO₃H; —SO₂H; —SO₂NH₂, —SSO₃H, —PO₃H₂; —NO₂, —SH, —OSO₃H, —OC(O)(OH), —O—, —S—, halomethyl, dihalomethyl, trihalomethyl, —SO₂N(X¹)X² or N(X¹)X², wherein X¹ and X² are each, independently, H, aryl, C₁-C₆-alkyl, esters thereof; salts thereof, and any combination thereof;
R¹, R², R³, R⁴, are each, independently, selected from the group consisting of —H, —OH, halogen, —(CH₂)₀₋₆Y, —O—(CH₂)₀₋₆Y, wherein Y is selected from —H, —CN, —CO₂H, —SO₃H, —SO₂H, —PO₃H₂, —NO₂, —SSO₃H, halomethyl, dihalomethyl, trihalomethyl, N-methyl-piperidinyl, morpholinyl, hydroxyphenyl, phenyl, piperazinyl, cyclopropyl, cyclopentyl, cyclohexyl, pyridinyl, 5H-tetrazolyl, triazolyl or piperidinyl; R⁹(CH₂)₀₋₆COO—, —N(R⁹)(CH₂)₀₋₆COO(R⁹), —O(CH₂)₀₋₆(R⁹), —(CH₂)₁₋₆COO(R⁹), —(CH₂)₁₋₆N(R⁹)COO(R⁹), —(CH₂)₁₋₆N(R⁹)CO(R⁹), —(CH₂)₁₋₆CONH(R⁹), ═NOR⁹, wherein R⁹ is selected from the group consisting of —H, —C₁₋₄-alkyl, N-methyl-piperidinyl, morpholinyl, hydroxyphenyl, phenyl, piperazinyl, cyclopropyl, cyclopentyl, cyclohexyl, pyridinyl, 5H-tetrazolyl, triazolyl, or piperidinyl; —N(X¹)X², —SO₂N(X¹)X² wherein X¹ and X² are each, independently, H, aryl, C₁-C₆-alkyl; salts thereof, esters thereof, and any combination thereof;
R¹ and R² can also form together for a fused 5- or 6-membered ring composed of one of the following bridging bivalent radicals (reading from R¹ to R²):
-GR¹⁰—CH₂—CH₂—
—CH₂-GR¹⁰—CH₂—
—CH₂—CH₂-GR¹⁰—
-GR¹⁰—CH₂—CH₂—CH₂—
—CH₂-GR¹⁰—CH₂—CH₂—
—CH₂—CH₂-GR¹⁰—CH₂—
—CH₂—CH₂—CH₂-GR¹⁰—
-GR¹⁰═CH—CH═CH—
—CH=GR¹⁰—CH═CH—
—CH═CH-GR¹⁰═CH—
—CH═CH—CH=GR¹⁰—
wherein G is either an sp²- or sp³-hybridized carbon or nitrogen atom; wherein R¹⁰ has the meaning set forth for R⁶;
a, b, c and d are each 0 or 1.

109-111. (canceled)

112. The method of claim 108, wherein the compound is of the Formula 2, 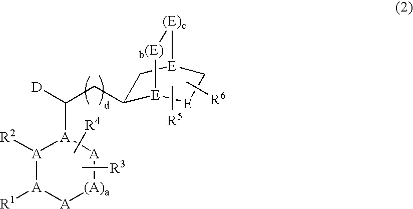
or a pharmaceutically acceptable salt thereof,
wherein
A and E are each, independently, an sp²- or sp³-hybridized carbon or nitrogen atom;
D is selected from the group consisting of —H, —OH, halogen, —(CH₂)₀₋₆Y, —O—(CH₂)₀₋₆Y, wherein Y is selected from —H, —CN, —CO₂H, —SO₃H, —SO₂H, —PO₃H₂, —NO₂, —SSO₃H, halomethyl, dihalomethyl, trihalomethyl, N-methyl-piperidinyl, morpholinyl, hydroxyphenyl, phenyl, piperazinyl, cyclopropyl, cyclopentyl, cyclohexyl, pyridinyl, 5H-tetrazolyl, triazolyl or piperidinyl; R⁹(CH₂)₀₋₆COO—, —N(R⁹)(CH₂)₀₋₆COO(R⁹), —O(CH₂)₀₋₆(R⁹), —(CH₂)₁₋₆COO(R⁹), —(CH₂)₁₋₆N(R⁹)COO(R⁹), —(CH₂)₁₋₆N(R⁹)CO(R), —(CH₂)₁₋₆CONH(R⁹), ═NOR⁹, wherein R⁹ is selected from the group consisting of —H, —C₁₋₄-alkyl, N-methyl-piperidinyl, morpholinyl, hydroxyphenyl, phenyl, piperazinyl, cyclopropyl, cyclopentyl, cyclohexyl, pyridinyl, 5H-tetrazolyl, triazolyl, or piperidinyl; —N(X¹)X², —SO₂N(X¹)X², wherein X¹ and X² are each, independently, H, aryl, C₁-C₆-alkyl; salts thereof, esters thereof, and any combination thereof;
R¹, R², R³, R⁴, R⁵ and R⁶, are each, independently, —H, —OH, halo, C₁-C₆-alkyl, —O—C₁₋₆-alkyl, —CHCH₂, —CH₂CHCH₂, —(CH₂)₀₋₆Y, —O—(CH₂)₀₋₆Y, wherein Y is selected from —H, —CN, —CO₂H, —SO₃H, —SO₂H, —PO₃H₂, —NO₂, —SSO₃H, halomethyl, dihalomethyl, trihalomethyl, N-methyl-piperidinyl, morpholinyl, hydroxyphenyl, phenyl, piperazinyl, cyclopropyl, cyclopentyl, cyclohexyl, pyridinyl, 5H-tetrazolyl, triazolyl or piperidinyl; R⁹(CH₂)₀₋₆COO—, —N(R⁹)(CH₂)₀₋₆COO(R⁹), —O(CH₂)₀₋₆(R⁹), —(CH₂)₁₋₆COO(R⁹), —(CH₂)₁₋₆N(R⁹)COO(R⁹), —(CH₂)₁₋₆N(R⁹)CO(R⁹), —(CH₂)₁₋₆CONH(R⁹), ═NOR⁹, wherein R⁹ is selected from the group consisting of —H, —C₁₋₄-alkyl, N-methyl-piperidinyl, morpholinyl, hydroxyphenyl, phenyl, piperazinyl, cyclopropyl, cyclopentyl, cyclohexyl, pyridinyl, 5H-tetrazolyl, triazolyl, or piperidinyl; —N(X¹)X², —SO₂N(X¹)X² wherein X¹ and X² are each, independently, H, aryl, C₁-C₆-alkyl; salts thereof, esters thereof, and any combination thereof;
R¹and R² can also form together for a fused 5- or 6-membered ring composed of one of the following bridging bivalent radicals (reading from R¹ to R²):
-GR¹⁰—CH₂—CH₂—
—CH₂-GR¹⁰—CH₂—
—CH₂—CH₂-GR¹⁰—
-GR¹⁰—CH₂—CH₂—CH₂—
—CH₂-GR¹⁰—CH₂—CH₂—
—CH₂—CH₂-GR¹⁰—CH₂—
—CH₂—CH₂—CH₂-GR¹⁰—
-GR¹⁰═CH—CH═CH—
—CH=GR¹⁰—CH═CH—
—CH═CH-GR¹⁰═CH—
—CH═CH—CH=GR¹⁰—
wherein G is either an sp²- or sp3-hybridized carbon or nitrogen atom; wherein R¹⁰ has the meaning set forth for R⁶;
a, b, c and d are each 0 or 1.

113-116. (canceled)

117. The method of claim 108, wherein the compound is of the Formula 3, 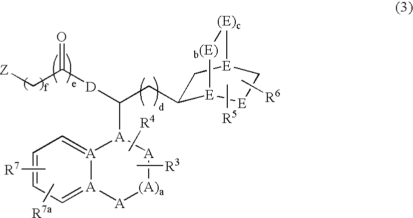
or a pharmaceutically acceptable salt thereof,
wherein
A and E are each, independently, an sp²- or sp³-hybridized carbon or nitrogen atom;
D is selected from the group consisting of —CH₂CHCH—, —CH₂CHCHCH₂—, —O—, —[(CH₂)₁₋₆]—, —O—(CH₂)₁₋₆—, —O—(CH₂)₁₋₆—N(R⁹)—, —(CH₂)₁₋₆—N(R⁹)—, N(R⁹)—, wherein R⁹ is selected from the group consisting of —H, —C₁₋₄-alkyl, N-methyl-piperidinyl, morpholinyl, hydroxyphenyl, phenyl, piperazinyl, cyclopropyl, cyclopentyl, cyclohexyl, pyridinyl, 5H-tetrazolyl, triazolyl or piperidinyl;
R³, R⁴, R⁵, R⁶, R⁷ and R^{7a} are each, independently, —H, —OH, —(CH₂)₀₋₆Y, —O—(CH₂)₀₋₆Y, wherein Y is selected from —H, —OH, —CN, halo, —CHCH₂, —CH₂CHCH₂, —NO₂, morpholinyl, hydroxyphenyl, phenyl, piperazinyl, cyclopropyl, cyclopentyl, cyclohexyl, pyridinyl, 5H-tetrazolyl, triazolyl, piperidinyl, alkylcarbonyl, alkylthiocarbonyl, alkoxycarbonyl, aminocarbonyl, —CO₂H; —SO₃H; —SO₂H; —SO₂NH₂, —SSO₃H, —PO₃H₂; —NO₂, —SH, —OSO₃H, —OC(O)(OH), —O—, —S—, halomethyl, dihalomethyl, trihalomethyl, —SO₂N(X¹)X² or N(X¹)X², wherein X¹ and X² are each, independently, H, aryl, C₁-C₆-alkyl, esters thereof; salts thereof, and any combination thereof;
Z is selected from the group consisting of —H, —OH, —(CH₂)₀₋₆Y, —O—(CH₂)₀₋₆Y, wherein Y is selected from —H, —OH, —CN, halo, —CHCH₂, —CH₂CHCH₂, —NO₂, morpholinyl, hydroxyphenyl, phenyl, piperazinyl, cyclopropyl, cyclopentyl, cyclohexyl, pyridinyl, 5H-tetrazolyl, triazolyl, piperidinyl, alkylcarbonyl, alkylthiocarbonyl, alkoxycarbonyl, aminocarbonyl, —CO₂H, —SO₃H; —SO₂H; —SO₂NH₂, —SSO₃H, —PO₃H₂; —NO₂, —SH, —OSO₃H, —OC(O)(OH), —O—, —S—, halomethyl, dihalomethyl, trihalomethyl, —SO₂N(X¹)X² or N(X¹)X², wherein X¹ and X² are each, independently, H, aryl, C₁-C₆-alkyl, esters thereof; salts thereof, and any combination thereof;
R⁷ and R^{7a} can also form together for a fused 5- or 6-membered ring composed of one of the following bridging bivalent radicals:
—O—CH₂—CH₂—
—CH₂—O—CH₂—
—CH₂—CH₂—O—
—CH₂—CH₂—CH₂—
—O—CH₂—O—
—CH₂—CH₂—CH₂—CH₂—
a, b, c, d and e are each 0 or 1
f is 0, 1, 2, 3, 4, 5 or 6.

118-127. (canceled)

128. The method of claim 108, wherein the compound is of the Formula 4, 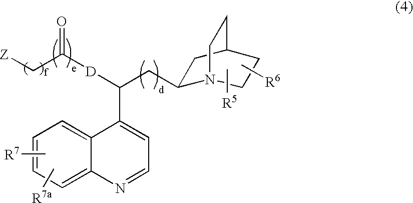
or a pharmaceutically acceptable salt thereof,
wherein
D is selected from the group consisting of —O—, —(CH₂)₁₋₆—, —N(R⁹)—, wherein R⁹ is selected from the group consisting of —H, —C₁₋₄-alkyl, N-methyl-piperidinyl, morpholinyl, hydroxyphenyl, phenyl, piperazinyl, cyclopropyl, cyclopentyl, cyclohexyl, pyridinyl, 5H-tetrazolyl, triazolyl, or piperidinyl;
R⁵, R⁶, R⁷ and R^{7a} are each, independently, —H, —OH, —(CH₂)₀₋₆Y, —O—(CH₂)₀₋₆Y, wherein Y is selected from —H, —OH, —CN, halo, —CHCH₂, —CH₂CHCH₂, —NO₂, morpholinyl, hydroxyphenyl, phenyl, piperazinyl, cyclopropyl, cyclopentyl, cyclohexyl, pyridinyl, 5H-tetrazolyl, triazolyl, piperidinyl, alkylcarbonyl, alkylthiocarbonyl, alkoxycarbonyl, aminocarbonyl, —CO₂H; —SO₃H; —SO₂H; —SO₂NH₂, —SSO₃H, —PO₃H₂; —NO₂, —SH, —OSO₃H, —OC(O)(OH), —O—, —S—, halomethyl, dihalomethyl, trihalomethyl, —SO₂N(X¹)X² or N(X¹)X², wherein X¹ and X² are each, independently, H, aryl, C₁-C₆-alkyl, esters thereof; salts thereof, and any combination thereof;
Z is selected from the group consisting of —H, —OH, —(CH₂)₀₋₆Y, —O—(CH₂)₀₋₆Y, wherein Y is selected from —H, —OH, —CN, halo, —CHCH₂, —CH₂CHCH₂, —NO₂, morpholinyl, hydroxyphenyl, phenyl, piperazinyl, cyclopropyl, cyclopentyl, cyclohexyl, pyridinyl, 5H-tetrazolyl, triazolyl, piperidinyl, alkylcarbonyl, alkylthiocarbonyl, alkoxycarbonyl, aminocarbonyl, —CO₂H, —SO₃H; —SO₂H; —SO₂NH₂, —SSO₃H, —PO₃H₂; —NO₂, —SH, —OSO₃H, —OC(O)(OH), —O—, —S—, halomethyl, dihalomethyl, trihalomethyl, —SO₂N(X¹)X² or N(X¹)X², wherein X¹ and X² are each, independently, H, aryl, C₁-C₆-alkyl, esters thereof; salts thereof, and any combination thereof;
R⁷ and R^{7a} can also form together for a fused 5- or 6-membered ring composed of one of the following bridging bivalent radicals:
—O—CH₂—CH₂—
—CH₂—O—CH₂—
—CH₂—CH₂—O—
—CH₂—CH₂—CH₂—
—O—CH₂—O—
—CH₂—CH₂—CH₂—CH₂—
d and e are each, independently, 0 or 1;
f is 0, 1, 2, 3, 4, 5 or 6.

129-138. (canceled)

139. The method of claim 108, wherein the compound is of the Formula 5, 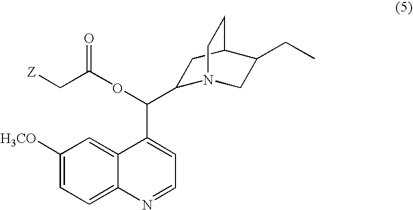
or a pharmaceutically acceptable salt thereof,
wherein
Z is selected from the group consisting of —H, —OH, —(CH₂)₀₋₆Y, —O—(CH₂)₀₋₆Y, wherein Y is selected from —H, C₁-C₆-alkyl, —OH, —CN, halo, —CHCH₂, —CH₂CHCH₂, —NO₂, morpholinyl, hydroxyphenyl, phenyl, piperazinyl, cyclopropyl, cyclopentyl, cyclohexyl, pyridinyl, 5H-tetrazolyl, triazolyl, piperidinyl, alkylcarbonyl, alkylthiocarbonyl, alkoxycarbonyl, aminocarbonyl, —CO₂H, —SO₃H; —SO₂H; —SO₂NH₂, —SSO₃H, —PO₃H₂; —NO₂, —SH, —OSO₃H, —OC(O)(OH), —O—, —S—, halomethyl, dihalomethyl, trihalomethyl, —SO₂N(X¹)X² or N(X¹)X², wherein X¹ and X² are each, independently, H, aryl, C₁-C₆-alkyl, esters thereof, salts thereof, and any combination thereof.

140-144. (canceled)

145. A method of treating a neurological disorder in a subject in need thereof, comprising administering an effective amount of a compound of Formula 1, 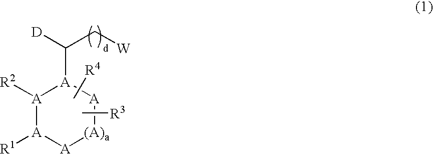
or a pharmaceutically acceptable salt thereof,
wherein
A is, independently, either an sp²- or sp³-hybridized carbon or nitrogen atom; D is selected from the group consisting of —H, —OH, halogen, —(CH₂)₀₋₆Y, —O—(CH₂)₀₋₆Y, wherein Y is selected from —H, —CN, —CO₂H, —SO₃H, —SO₂H, —PO₃H₂, —NO₂, —SSO₃H, halomethyl, dihalomethyl, trihalomethyl, N-methyl-piperidinyl, morpholinyl, hydroxyphenyl, phenyl, piperazinyl, cyclopropyl, cyclopentyl, cyclohexyl, pyridinyl, 5H-tetrazolyl, triazolyl or piperidinyl; R⁹(CH₂)₀₋₆COO—, —N(R⁹)(CH₂)₀₋₆COO(R⁹), —O(CH₂)₀₋₆(R⁹), —(CH₂)₁₋₆COO(R⁹), —(CH₂)₁₋₆N(R⁹)COO(R⁹), —(CH₂)₁₋₆N(R⁹)CO(R⁹), —(CH₂)₁₋₆CONH(R⁹), ═NOR⁹, wherein R⁹ is selected from the group consisting of —H, —C₁₋₄-alkyl, N-methyl-piperidinyl, morpholinyl, hydroxyphenyl, phenyl, piperazinyl, cyclopropyl, cyclopentyl, cyclohexyl, pyridinyl, 5H-tetrazolyl, triazolyl, or piperidinyl; —N(X¹)X², —SO₂N(X¹)X², wherein X¹ and X² are each, independently, H, aryl, or C₁-C₆-alkyl; salts thereof, esters thereof, and any combination thereof;
W is selected from the group consisting of —H, —OH, halogen, —(CH₂)₀₋₆Y, —O—(CH₂)₀₋₆Y, wherein Y is selected from —H, —CN—CO₂H, —SO₃H, —SO₂H, —PO₃H₂, —NO₂, —SSO₃H, halomethyl, dihalomethyl, trihalomethyl, N-methyl-piperidinyl, morpholinyl, hydroxyphenyl, phenyl, piperazinyl, cyclopropyl, cyclopentyl, cyclohexyl, pyridinyl, 5H-tetrazolyl, triazolyl or piperidinyl; R⁹(CH₂)₀₋₆COO—, —N(R⁹)(CH₂)₀₋₆COO(R⁹), —O(CH₂)₀₋₆(R⁹), —(CH₂)₁₋₆COO(R⁹), —(CH₂)₁₋₆N(R⁹)COO(R⁹), —(CH₂)₁₋₆N(R⁹)CO(R⁹), —(CH₂)₁₋₆CONH(R⁹), ═NOR⁹, wherein R⁹ is selected from the group consisting of —H, —C₁₋₄-alkyl, N-methyl-piperidinyl, morpholinyl, hydroxyphenyl, phenyl, piperazinyl, cyclopropyl, cyclopentyl, cyclohexyl, pyridinyl, 5H-tetrazolyl, triazolyl, or piperidinyl; —N(X¹)X², —SO₂N(X¹)X², or —(CH₂)₁₋₆SO₂N(X¹)X², wherein X¹ and X² are each, independently, H, aryl, C₁-C₆-alkyl; salts thereof, esters thereof, and any combination thereof; and any combination thereof; or the formula 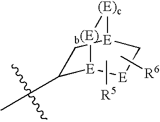
wherein E is, independently, either an sp²- or sp³-hybridized carbon or nitrogen atom; R⁵ and R⁶ are each, independently, —H, —OH, —(CH₂)₀₋₆Y, —O—(CH₂)₀₋₆Y, wherein Y is selected from —H, —OH, —CN, halo, —CHCH₂, —CH₂CHCH₂, —NO₂, morpholinyl, hydroxyphenyl, phenyl, piperazinyl, cyclopropyl, cyclopentyl, cyclohexyl, pyridinyl, 5H-tetrazolyl, triazolyl, piperidinyl, alkylcarbonyl, alkylthiocarbonyl, alkoxycarbonyl, aminocarbonyl, —CO₂H; —SO₃H; —SO₂H; —SO₂NH₂, —SSO₃H, —PO₃H₂; —NO₂, —SH, —OSO₃H, —OC(O)(OH), —O—, —S—, halomethyl, dihalomethyl, trihalomethyl, —SO₂N(X¹)X² or N(X¹)X², wherein X¹ and X² are each, independently, H, aryl, C₁-C₆-alkyl, esters thereof; salts thereof, and any combination thereof;
R¹, R², R³, R⁴, are each, independently, selected from the group consisting of —H, —OH, halogen, —(CH₂)₀₋₆Y, —O—(CH₂)₀₋₆Y, wherein Y is selected from —H, —CN, —CO₂H, —SO₃H, —SO₂H, —PO₃H₂, —NO₂, —SSO₃H, halomethyl, dihalomethyl, trihalomethyl, N-methyl-piperidinyl, morpholinyl, hydroxyphenyl, phenyl, piperazinyl, cyclopropyl, cyclopentyl, cyclohexyl, pyridinyl, 5H-tetrazolyl, triazolyl or piperidinyl; R⁹(CH₂)₀₋₆COO—, —N(R⁹)(CH₂)₀₋₆COO(R⁹), —O(CH₂)₀₋₆(R⁹), —(CH₂)₁₋₆COO(R⁹), —(CH₂)₁₋₆N(R⁹)COO(R⁹), —(CH₂)₁₋₆N(R⁹)CO(R⁹), —(CH₂)₁₋₆CONH(R⁹), ═NOR⁹, wherein R⁹ is selected from the group consisting of —H, —C₁₋₄-alkyl, N-methyl-piperidinyl, morpholinyl, hydroxyphenyl, phenyl, piperazinyl, cyclopropyl, cyclopentyl, cyclohexyl, pyridinyl, 5H-tetrazolyl, triazolyl, or piperidinyl; —N(X¹)X², —SO₂N(X¹)X² wherein X¹ and X² are each, independently, H, aryl, C₁-C₆-alkyl; salts thereof, esters thereof, and any combination thereof;
R¹ and R² can also form together for a fused 5- or 6-membered ring composed of one of the following bridging bivalent radicals (reading from R¹ to R²):
-GR¹⁰—CH₂—CH₂—
—CH₂-GR¹⁰—CH₂—
—CH₂—CH₂-GR¹⁰—
-GR¹⁰—CH₂—CH₂—CH₂—
—CH₂-GR¹⁰—CH₂—CH₂—
—CH₂—CH₂-GR¹⁰—CH₂—
—CH₂—CH₂—CH₂-GR¹⁰—
-GR¹⁰═CH—CH═CH—
—CH=GR¹⁰—CH═CH—
—CH═CH-GR¹⁰═CH—
—CH═CH—CH=GR¹⁰—
wherein G is either an sp²- or sp³-hybridized carbon or nitrogen atom; wherein R¹⁰ has the meaning set forth for R⁶;
a, b, c and d are each 0 or 1.

146-148. (canceled)

149. The method of claim 145, wherein the compound of the Formula 2, 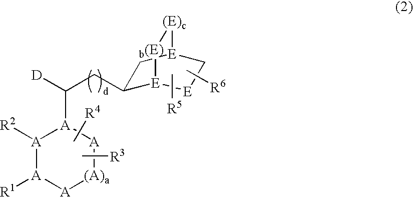
or a pharmaceutically acceptable salt thereof,
wherein
A and E are each, independently, an sp²- or sp³-hybridized carbon or nitrogen atom;
D is selected from the group consisting of —H, —OH, halogen, —(CH₂)₀₋₆Y, —O—(CH₂)₀₋₆Y, wherein Y is selected from —H, —CN, —CO₂H, —SO₃H, —SO₂H, —PO₃H₂, —NO₂, —SSO₃H, halomethyl, dihalomethyl, trihalomethyl, N-methyl-piperidinyl, morpholinyl, hydroxyphenyl, phenyl, piperazinyl, cyclopropyl, cyclopentyl, cyclohexyl, pyridinyl, 5H-tetrazolyl, triazolyl or piperidinyl; R⁹(CH₂)₀₋₆COO—, —N(R⁹)(CH₂)₀₋₆COO(R⁹), —O(CH₂)₀₋₆(R⁹), —(CH₂)₁₋₆COO(R⁹), —(CH₂)₁₋₆N(R⁹)COO(R⁹), —(CH₂)₁₋₆N(R⁹)CO(R⁹), —(CH₂)₁₋₆CONH(R⁹), ═NOR⁹, wherein R⁹ is selected from the group consisting of —H, —C₁₋₄-alkyl, N-methyl-piperidinyl, morpholinyl, hydroxyphenyl, phenyl, piperazinyl, cyclopropyl, cyclopentyl, cyclohexyl, pyridinyl, 5H-tetrazolyl, triazolyl, or piperidinyl; —N(X¹)X², —SO₂N(X¹)X², wherein X¹ and X² are each, independently, H, aryl, C₁-C₆-alkyl; salts thereof, esters thereof, and any combination thereof,
R¹, R², R³, R⁴, R⁵ and R⁶, are each, independently, —H, —OH, halo, C₁-C₆-alkyl, —O—C₁₋₆-alkyl, —CHCH₂, —CH₂CHCH₂, —(CH₂)₀₋₆Y, —O—(CH₂)₀₋₆Y, wherein Y is selected from —H, —CN, —CO₂H, —SO₃H, —SO₂H, —PO₃H₂, —NO₂, —SSO₃H, halomethyl, dihalomethyl, trihalomethyl, N-methyl-piperidinyl, morpholinyl, hydroxyphenyl, phenyl, piperazinyl, cyclopropyl, cyclopentyl, cyclohexyl, pyridinyl, 5H-tetrazolyl, triazolyl or piperidinyl; R⁹(CH₂)₀₋₆COO—, —N(R⁹)(CH₂)₀₋₆COO(R⁹), —O(CH₂)₀₋₆(R⁹), —(CH₂)₁₋₆COO(R⁹), —(CH₂)₁₋₆N(R⁹)COO(R⁹), —(CH₂)₁₋₆N(R⁹)CO(R⁹), —(CH₂)₁₋₆CONH(R⁹), ═NOR⁹, wherein R⁹ is selected from the group consisting of —H, —C₁₋₄-alkyl, N-methyl-piperidinyl, morpholinyl, hydroxyphenyl, phenyl, piperazinyl, cyclopropyl, cyclopentyl, cyclohexyl, pyridinyl, 5H-tetrazolyl, triazolyl, or piperidinyl; —N(X¹)X², —SO₂N(X¹)X² wherein X¹ and X² are each, independently, H, aryl, C₁-C₆-alkyl; salts thereof, esters thereof, and any combination thereof;
R¹ and R² can also form together for a fused 5- or 6-membered ring composed of one of the following bridging bivalent radicals (reading from R¹ to R²):
-GR¹⁰—CH₂—CH₂—
—CH₂-GR¹⁰—CH₂—
—CH₂—CH₂-GR¹⁰—
-GR¹⁰—CH₂—CH₂—CH₂—
—CH₂-GR¹⁰—CH₂—CH₂—
—CH₂—CH₂-GR¹⁰—CH₂—
—CH₂—CH₂—CH₂-GR¹⁰—
-GR¹⁰═CH—CH═CH—
—CH=GR¹⁰—CH═CH—
—CH═CH-GR¹⁰═CH—
—CH═CH—CH=GR¹⁰—
wherein G is either an sp²- or sp³-hybridized carbon or nitrogen atom; wherein R¹⁰ has the meaning set forth for R⁶;
a, b, c and dare each 0 or 1.

150-153. (canceled)

154. The method of claim 145, wherein the compound of the Formula 3, 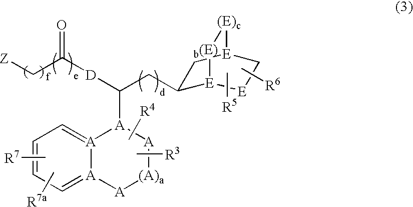
or a pharmaceutically acceptable salt thereof,
wherein
A and E are each, independently, an sp²- or sp³-hybridized carbon or nitrogen atom;
D is selected from the group consisting of —CH₂CHCH—, —CH₂CHCHCH₂—, —O—, —[(CH₂)₁₋₆]—, —O—(CH₂)₁₋₆—, —O—(CH₂)₁₋₆—N(R⁹)—, —(CH₂)₁₋₆—N(R⁹)-—N(R⁹)—, wherein R⁹ is selected from the group consisting of —H, —C₁₋₄-alkyl, N-methyl-piperidinyl, morpholinyl, hydroxyphenyl, phenyl, piperazinyl, cyclopropyl, cyclopentyl, cyclohexyl, pyridinyl, 5H-tetrazolyl, triazolyl or piperidinyl;
R³, R⁴, R⁵, R⁶, R⁷ and R^{7a} are each, independently, —H, —OH, —(CH₂)₀₋₆Y, —O—(CH₂)₀₋₆Y, wherein Y is selected from —H, —OH, —CN, halo, —CHCH₂, —CH₂CHCH₂, —NO₂, morpholinyl, hydroxyphenyl, phenyl, piperazinyl, cyclopropyl, cyclopentyl, cyclohexyl, pyridinyl, 5H-tetrazolyl, triazolyl, piperidinyl, alkylcarbonyl, alkylthiocarbonyl, alkoxycarbonyl, aminocarbonyl, —CO₂H; —SO₃H; —SO₂H; —SO₂NH₂, —SSO₃H, —PO₃H₂; —NO₂, —SH, —OSO₃H, —OC(O)(OH), —O—, —S—, halomethyl, dihalomethyl, trihalomethyl, —SO₂N(X¹)X² or N(X¹)X², wherein X¹ and X² are each, independently, H, aryl, C₁-C₆-alkyl, esters thereof; salts thereof, and any combination thereof;
Z is selected from the group consisting of —H, —OH, —(CH₂)₀₋₆Y, —O—(CH₂)₀₋₆Y, wherein Y is selected from —H, —OH, —CN, halo, —CHCH₂, —CH₂CHCH₂, —NO₂, morpholinyl, hydroxyphenyl, phenyl, piperazinyl, cyclopropyl, cyclopentyl, cyclohexyl, pyridinyl, 5H-tetrazolyl, triazolyl, piperidinyl, alkylcarbonyl, alkylthiocarbonyl, alkoxycarbonyl, aminocarbonyl, —CO₂H, —SO₃H; —SO₂H; —SO₂NH₂, —SSO₃H, —PO₃H₂; —NO₂, —SH, —OSO₃H, —OC(O)(OH), —O—, —S—, halomethyl, dihalomethyl, trihalomethyl, —SO₂N(X¹)X² or N(X¹)X², wherein X¹ and X² are each, independently, H, aryl, C₁-C₆-alkyl, esters thereof; salts thereof, and any combination thereof;
R⁷ and R^{7a} can also form together for a fused 5- or 6-membered ring composed of one of the following bridging bivalent radicals:
—O—CH₂—CH₂—
—CH₂—O—CH₂—
—CH₂—CH₂—O—
—CH₂—CH₂—CH₂—
—O—CH₂—O—
—CH₂—CH₂—CH₂—CH₂—
a, b, c, d and e are each 0 or 1
f is 0, 1, 2, 3, 4, 5 or 6.

155-164. (canceled)

165. The method of claim 145, wherein the compound of the Formula 4, 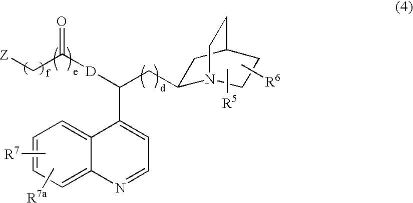
or a pharmaceutically acceptable salt thereof,
wherein
D is selected from the group consisting of —O—, —(CH₂)₁₋₆—, —N(R⁹)—, wherein R⁹ is selected from the group consisting of —H, —C₁₋₄-alkyl, N-methyl-piperidinyl, morpholinyl, hydroxyphenyl, phenyl, piperazinyl, cyclopropyl, cyclopentyl, cyclohexyl, pyridinyl, 5H-tetrazolyl, triazolyl, or piperidinyl;
R⁵, R⁶, R⁷ and R^{7a} are each, independently, —H, —OH, —(CH₂)₀₋₆Y, —O—(CH₂)₀₋₆Y, wherein Y is selected from —H, —OH, —CN, halo, —CHCH₂, —CH₂CHCH₂, —NO₂, morpholinyl, hydroxyphenyl, phenyl, piperazinyl, cyclopropyl, cyclopentyl, cyclohexyl, pyridinyl, 5H-tetrazolyl, triazolyl, piperidinyl, alkylcarbonyl, alkylthiocarbonyl, alkoxycarbonyl, aminocarbonyl, —CO₂H; —SO₃H; —SO₂H; —SO₂NH₂, —SSO₃H, —PO₃H₂; —NO₂, —SH, —OSO₃H, —OC(O)(OH), —O—, —S—, halomethyl, dihalomethyl, trihalomethyl, —SO₂N(X¹)X² or N(X¹)X², wherein X¹ and X² are each, independently, H, aryl, C₁-C₆-alkyl, esters thereof; salts thereof, and any combination thereof;
Z is selected from the group consisting of —H, —OH, —(CH₂)₀₋₆Y, —O—(CH₂)₀₋₆Y, wherein Y is selected from —H, —OH, —CN, halo, —CHCH₂, —CH₂CHCH₂, —NO₂, morpholinyl, hydroxyphenyl, phenyl, piperazinyl, cyclopropyl, cyclopentyl, cyclohexyl, pyridinyl, 5H-tetrazolyl, triazolyl, piperidinyl, alkylcarbonyl, alkylthiocarbonyl, alkoxycarbonyl, aminocarbonyl, —CO₂H, —SO₃H; —SO₂H; —SO₂NH₂, —SSO₃H, —PO₃H₂; —NO₂, —SH, —OSO₃H, —OC(O)(OH), —O—, —S—, halomethyl, dihalomethyl, trihalomethyl, —SO₂N(X¹)X² or N(X¹)X², wherein X¹ and X² are each, independently, H, aryl, C₁-C₆-alkyl, esters thereof; salts thereof, and any combination thereof;
R⁷ and R^{7a} can also form together for a fused 5- or 6-membered ring composed of one of the following bridging bivalent radicals:
—O—CH₂—CH₂—
—CH₂—O—CH₂—
—CH₂—CH₂—O—
—CH₂—CH₂—CH₂—
—O—CH₂—O—
—CH₂—CH₂—CH₂—CH₂—
d and e are each, independently, 0 or 1;
f is 0, 1, 2, 3, 4, 5 or 6.

166-175. (canceled)

176. The method of claim 145, wherein the compound of the Formula 5, 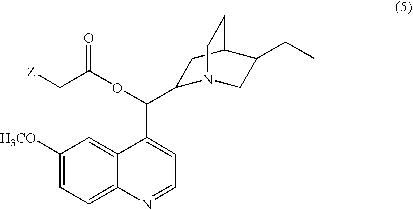
or a pharmaceutically acceptable salt thereof,
wherein
Z is selected from the group consisting of —H, —OH, —(CH₂)₀₋₆Y, —O—(CH₂)₀₋₆Y, wherein Y is selected from —H, C₁-C₆-alkyl, —OH, —CN, halo, —CHCH₂, —CH₂CHCH₂, —NO₂, morpholinyl, hydroxyphenyl, phenyl, piperazinyl, cyclopropyl, cyclopentyl, cyclohexyl, pyridinyl, 5H-tetrazolyl, triazolyl, piperidinyl, alkylcarbonyl, alkylthiocarbonyl, alkoxycarbonyl, aminocarbonyl, —CO₂H, —SO₃H; —SO₂H; —SO₂NH₂, —SSO₃H, —PO₃H₂; —NO₂, —SH, —OSO₃H, —OC(O)(OH), —O—, —S—, halomethyl, dihalomethyl, trihalomethyl, —SO₂N(X¹)X² or N(X¹)X², wherein X¹ and X² are each, independently, H, aryl, C₁-C₆-alkyl, esters thereof; salts thereof, and any combination thereof.

177-183. (canceled)

184. The method of claim 70, wherein the method further comprises administering an adjuvant composition.

185. The method of claim 108, wherein the method further comprises administering an adjuvant composition.

186. The method of claim 145, wherein the method further comprises administering an adjuvant composition.
